(19) United States
(12) Patent Application Publication
(10) Pub. No.: US 2023/0132250 A1
(43) Pub. Date: Apr. 27, 2023

(54) BACTERIAL HOST STRAINS

(71) Applicant: Nature Technology Corporation, Lincoln, NE (US)

(72) Inventor: James A. Williams, Lincoln, NE (US)

(73) Assignee: Nature Technology Corporation, Lincoln, NE (US)

(21) Appl. No.: 17/931,000

(22) Filed: Sep. 9, 2022

Related U.S. Application Data
(63) Continuation of application No. PCT/US21/22002, filed on Mar. 11, 2021.
(60) Provisional application No. 62/988,223, filed on Mar. 11, 2020.

(51) Int. Cl. C12N 15/86

(52) U.S. Cl. CPC C12N 15/86; C12N 2750/14143; C12N 2750/14152

(57) ABSTRACT

The present disclosure provides engineered E. coli host cells that combine a knockout of SbcC, SbcD, or both without certain other mutations that can be used to propogate vectors. Methods of improved vector production using such engineered E. coli host cells are also provided.

### CROSS-REFERENCE TO RELATED APPLICATIONS

This application is a Continuation of International Application No. PCT/US2021/022002, which was filed Mar. 11, 2021, the entire contents of which are hereby incorporated herein by reference in their entirety. International Application No. PCT/US2021/022002 claims priority to U.S. Provisional Patent Application Ser. No. 62/988,223, entitled “Bacterial Host Strains” which was filed Mar. 11, 2020, the entire contents of which are incorporated herein by reference.

### SEQUENCE LISTING

The instant application contains a Sequence Listing which has been submitted electronically in XML format and is hereby incorporated by reference in its entirety. Said XML copy, created on Sep. 9, 2022, is named 85535-372254_SL.xml and is 133,108 bytes in size. A corrected Sequence Listing was submitted electronically in XML format on Feb. 14, 2023. Said corrected copy, created on Jan. 15, 2023, is named “85535-372254_ST26.xml” and is 140,821 bytes in size.

### INCORPORATION BY REFERENCE

WO 2008/153733, WO 2014/035457 AND WO 2019/183248 are incorporated by reference herein in their entirety. Moreover, all publications, patents and patent application publications referenced herein are incorporated by reference herein in their entirety.

### BACKGROUND OF THE INVENTION

Escherichia coli (E. coli) plasmids have long been an important source of recombinant DNA molecules used by researchers and by industry. Today, plasmid DNA is becoming increasingly important as the next generation of biotechnology products (e.g., gene medicines and DNA vaccines) make their way into clinical trials, and eventually into the pharmaceutical marketplace. Plasmid DNA vaccines may find application as preventive vaccines for viral, bacterial, or parasitic diseases; immunizing agents for the preparation of hyper immune globulin products; therapeutic vaccines for infectious diseases; or as cancer vaccines. Plasmids are also utilized in gene therapy or gene replacement applications, wherein the desired gene product is expressed from the plasmid after administration to a patient. Plasmids are also utilized in non-viral transposon (e.g., Sleeping Beauty, PiggyBac, TCBuster, etc) vectors for gene therapy or gene replacement applications, wherein the desired gene product is expressed from the genome after transposition from the plasmid and genome integration. Plasmids are also utilized in Gene Editing (e.g., Homology-Directed Repair (HDR)/CRISPR-Cas9) non-viral vectors for gene therapy or gene replacement applications, wherein the desired gene product is expressed from the genome after excision from the plasmid and genome integration. Plasmids are also utilized in viral vectors (e.g., AAV, Lentiviral, retroviral vectors) for gene therapy or gene replacement applications, wherein the desired gene product is packaged in a transducing virus particle after transfection of a production cell line, and is then expressed from the virus in a target cell after viral transduction.

Non-viral and viral vector plasmids typically contain a pMB1-, ColE1- or pBR322-derived replication origin. Common high copy number derivatives have mutations affecting copy number regulation, such as ROP (Repressor of primer gene) deletion and a second site mutation that increases copy number (e.g., pMB1 pUC G to A point mutation, or ColE1 pMM1). Higher temperature (42° C.) can be employed to induce selective plasmid amplification with pUC and pMM1 replication origins.

WO2014/035457 discloses minimalized vectors (Nanoplasmid™) that utilize RNA-OUT antibiotic-free selection and replace the large 1000 bp pUC replication origin with a novel, 300 bp, R6K origin. Reduction of the spacer region linking the 5′ and 3′ ends of the transgene expression cassette to <500 bp with R6K origin-RNA-OUT backbones improves expression level compared to conventional minicircle DNA vectors.

U.S. Pat. No. 7,943,377, which is incorporated herein by reference in its entirety, describes methods for fed-batch fermentation, in which plasmid-containing E. coli cells were grown at a reduced temperature during part of the fed-batch phase, during which growth rate was restricted, followed by a temperature up-shift and continued growth at elevated temperature in order to accumulate plasmid; the temperature shift at restricted growth rate improved plasmid yield and purity. This fermentation process is herein referred to as the HyperGRO fermentation process. Other fermentation processes for plasmid production are described in Carnes A. E. 2005 BioProcess Intl 3:36-44, which is incorporated herein by reference in its entirety.

WO2014/035457 also discloses host strains for R6K origin vector production in the HyperGRO fermentation process.

Schnödt et al., (2016) Mol Ther—Nucleic Acids 5 e355, along with Chadeuf et al., (2005) Molecular Therapy 12:744-53 and Gray, 2017. WO2017/066579 teach that AAV helper plasmid antibiotic resistance markers are packaged into viral particles, demonstrating need to remove antibiotic markers from AAV helper plasmids as well as the AAV vector. There is no antibiotic marker transfer with the antibiotic free Nanoplasmid™ vectors disclosed in WO2014/035457.

Viral vectors such as AAV contain palindromic inverted terminal repeats (ITRs) DNA sequences at their termini.

Palindromes and inverted repeats are inherently unstable in high yield E. coli manufacturing hosts such as DH1, DH5α, JM107, JM108, JM109, XL1Blue and the like.

Growth of AAV ITR containing vectors is recommended to be performed in multiply mutant sbcC knockout cell lines SURE (a recB derivative of SRB) or SURE2.

The SURE cell line has the following genotype: F′[proAB⁺ lac I^{q} lacZΔM15 Tn10 (Tet^{R}] endA1 glnV44 thi-1 gyrA96 relA1 lac recB recJ sbcC umuC::Tn5 Kan^{R} uvrC e14⁻ (mcrA⁻) Δ(mcrCB-hsdSMR-mrr)171, where the SURE stabilizing mutations include sbcC in combination with recB recJ umuC uvrC ⁻(mcrA⁻) mcrBC-hsd-mrr.

The SRB cell line has the following genotype: F′[proAB⁺ lacI^{q} lacZΔM15 endA1 glnV44 thi-1 gyrA96 relA1 lac recJ sbcC umuC::Tn5(Kan^{R} uvrC e14⁻(mcrA⁻) Δ(mcrCB-hsdSMR-mrr)171, where the SRB stabilizing mutations include sbcC in combination with recJ umuC uvrC ⁻(mcrA⁻) mcrBC-hsd-mrr.

The SURE2 cell line has the following genotype: endA1 glnV44 thi-1 gyrA96 relA1 lac recB recJ sbcC umuC::Tn5 Kan^{R} uviC e14− Δ(mcrCB-hsdSMR-mur)171 F′[proAB⁺ lacI⁹ lacZΔM15 Tn10 (Tet^{R}) Amy Cm^{R}], where the SURE2 stabilizing mutations include sbcC in combination with recB recJ uvrC ⁻(mcrA⁻) mcrBC-hsd-mrr.

SbcCD is a nuclease that cleaves palindromic DNA sequences and contributes to palindrome instability in E. coli (Chalker A F, Leach D R, Lloyd R G. 1988 Gene 71:201-5). Palindromes such as shRNA or AAV ITRs are more stable in SbcC knockout strains such as SURE cells than DH5α as taught in Gray S J, Choi, V W, Asokan, A, Haberman R A, McCown T J, Samulski R J (2011) Curr Protoc Neurosci Chapter 4:Unit 4.17 as follows “The AAV ITRs are unstable in E. coli, and plasmids that lose the ITRs have a replication advantage in transformed cells. For these reasons, bacteria containing ITR plasmids should not be grown longer than 12-14 hours, and any recovered plasmids should be assessed for retention of the ITRs . . . . DH10B competent cells (or other comparable high-efficiency strain) can be used to transform ligation reactions for ITR-containing plasmid cloning. After screening positive clones for ITR integrity, a good clone should then be transformed into SURE or SURE2 cells (Agilent Technologies) for production of plasmid and glycerol stocks. SURE cells are engineered to maintain irregular DNA structures, but have lower transformation efficiency compared to DH10B.” Further, Siew S M, 2014 Recombinant AAV-mediated Gene Therapy Approaches to Treat Progressive Familial Intrahepatic Cholestasis Type 3. Thesis University of Sydney uploaded 2014-12-03 teaches “SURE2 cells are a sbcC mutant strain commonly used to propagate plasmids containing palindromic AAV ITRs.” Thus, it is generally understood that the SURE or SURE2 sbcC mutant strains are preferred to propagate plasmids containing palindromic AAV ITRs.

However, there are limitations to SURE or SURE2 cell lines. For example, SURE and SURE2 are kan^{R}, so they cannot be used to produce kanamycin resistance plasmids which are typically used (rather than ampicillin resistance plasmids) in cGMP manufacturing. Further, the art teaches that sbcC knockout stabilization of palindromes additionally requires mutations in other genes such as recB recJ uvrC mcrA, or mcrBC-hsd-mrr. Doherty J P, Lindeman R, Trent R J, Graham M W, Woodcock D M. 1993. Gene 124:29-35 report that not all palindromes are stabilized in SURE (or related SRB cell line). They recommended additional mutation (recC) are needed for palindrome stabilization as follows “However, while the palindrome-containing phage plated with reasonable efficiency on SURE (recB sbcC recJ umuC uvrC) and SRB (sbcC recJ umuC uvrC), the majority of phage recovered from these strains no longer required an sbcC host for subsequent plating. These two strains also gave poorer titers with a low-yielding phage clone from the human Prader-Willi chromosome region. Optimal phage hosts appear to be those that are mcrA delta(mcrBC-hsd-mrr) combined with mutations in sbcC plus recBC or recD.”

Consistent with this, other SbcC host strains also contain additional mutations, for example: PMC103: mcrA Δ(mcrBC-hsdRMS-mrr) 102 recD sbcC, where the PMC103 stabilizing mutations include sbcC in combination with recD (mcrA⁻) mcrBC-hsd-mrr; and PMC107: mcrA Δ (mcrBC-hsdRMS-mrr)102 recB21 recC22 recJ154 sbcB15 sbcC201, where the PMC107 stabilizing mutations include sbcC in combination with recB recJ sbcB (mcrA⁻) mcrBC-hsd-mrr.

Thus the art teaches that sbcC knockout stabilization of palindromes additionally requires mutations in sbcB, recB, recD, and recJ and, in some instances, uvrC, mcrA and/or mcrBC-hsd-mrr. This teaches away from application of sbcC knockout to improve palindrome stability in standard E. coli plasmid production strains such as DH1, DH5α, JM107, JM108, JM109, XL1Blue which do not contain these additional mutations.

For example, the genotypes of several standard E. coli plasmid production strains are:
DH1: F⁻ λ⁻ endA1 recA1 relA1 gyrA96 thi-1 glnV44 hsdR17(r_{K}⁻ m_{K}⁻)
DH5α: F− φ801acZΔM15 Δ(lacZYA-argF) U169 recA1 endA1 hsdR17 (rₖ−, mₖ+) gal-phoA supE44λ- thi-1 gyrA96 relA1
JM107: endA1 glnV44 thi-1 relA1 gyrA96 Δ(lac-proAB) [F traD36 proAB⁺ lacI^{q} lacZΔM15] hsdR17(R_{K}⁻ m_{K}⁺)) λ⁻
JM108: endA1 recA1 gyrA96 thi-1 relA1 glnV44 Δ(lac-proAB) hsdR17 (r_{K}⁻ m_{K}⁺)
JM109: endA1 glnV44 thi-1 relA1 gyrA96 recA1 mcrB⁻ Δ(lac-proAB) e14− [F′ traD36 proAB⁺ lacI^{q} lacZΔM15] hsdR17(r_{K}⁻ m_{K}⁺)
MG1655 K-12 F⁻ λ⁻ ilvG⁻ rfb-50 rph-1
XL1Blue: endA1 gyrA96(nal^{R}) thi-1 recA1 relA1 lac glnV44 F[::Tn10 proAB⁺ lacI^{q} Δ(lacZ)M15] hsdR17(r_{K}⁻ m_{K}⁺)

Standard E. coli plasmid production strains are endA, recA. However standard production strains do not contain any of the required mutations in sbcB, recB recD, and recJ and, in some instances, uvrC, mcrA, or mcrBC-hsd-mrr, so knockout of sbcC would not be expected to effectively stabilize palindromes or inverted repeats in the absence of these additional mutations.

However, the presence of multiple mutations in SURE and SURE2 cell lines decreases the viability of the cell lines and their productivity in E. coli fermentation plasmid production processes. For example, Table 1 summarizes HyperGRO fermentation plasmid yield and quality in SURE2 or XL1Blue (an example high yield E. coli manufacturing host). All three plasmids were low yielding and multimerization prone in SURE2, but high yielding (2-4×) and high quality (low multimerization) in XL1Blue.

TABLE 1
HyperGRO fermentation plasmid yields in SURE2
versus XL1Blue using ampR pUC origin plasmids

| | Sure2 Harvest | | XL1Blue Harvest | |
|---|---|---|---|---|
| | plasmid Yield | Sure2 Harvest | plasmid Yield | XL1Blue Harvest |
| Plasmid | (mg/L) | plasmid quality | (mg/L) | plasmid quality |
| Plasmid 1 | Ferm 1: 215 | CCC Multimer: | Ferm: 1113 | CCC Monomer |
| | Ferm 2: 251 | Monomer:dimer | | |
| | | mix | | |
| Plasmid 2 | Ferm 1: 248 | CCC Multimer: | Ferm: 893 | CCC Monomer |
| | Ferm 2: 378 | Monomer:dimer | | |
| | | mix | | |
| Plasmid 3 | Ferm 1: 341 | CCC Multimer: | Ferm: 578 | CCC Monomer |
| | Ferm 2: 293 | Monomer:dimer | | |
| | | mix | | |

*Methods for culture were the same as in the Examples below with the following temperature shifts: Sure 2: 30° C., Shift to 37° C. at 60 OD600, for 4 hr, 25° C. Hold; XL1Blue: 30° C., Shift to 42° C. at 55OD600, for 7 hr, 25° C. Hold.

Reduced viability and productivity are a common feature of multiply mutation ‘stabilizing hosts’, such as, for example Stbl2, Stbl3, and Stbl4 which are used to stabilize direct repeat containing vectors such as lentiviral vectors but do not contain the SbcC knockout. The genotypes of Stbl2, Stbl3 and Stbl4 are shown below.
Stbl2: F− endA1 glnV44 thi-1 recA1 gyrA96 relA1 Δ(lac-proAB) mcrA Δ(mcrBC-hsdRMS-mrr)) λ⁻
Stbl2 stabilizing mutations=mcrA Δ(mcrBC-hsdRMS-mrr) (Trinh, T., Jessee, J., Bloom, F. R., and Hirsch, V. (1994) FOCUS 16, 78.)
Stbl3: F− mcrB mrr hsdS20 (rB−, mB−) recA13 supE44 ara-14 galK2 lacY1 proA2 rpsL20 (Strr) xyl-5-leu mtl-1
Stbl3 stabilizing mutations=mcrBC−mrr
Stbl4: endA1 glnV44 thi-1 recA1 gyrA96 relA1 Δ(lac-proAB) mcrA Δ(mcrBC-hsdRMS-mrr)) λ⁻ gal F[proAB⁺ lacI^{q} lacZΔM15 Tn10]
Stbl4 stabilizing mutations=mcrA Δ(mcrBC-hsdRMS-mrr)

Therefore, there is a need for high yield E. coli production strains for high yield manufacture of palindrome- and inverted repat-containing vectors without ITR deletion or rearrangement which do not suffer from low stability or low viability.

### SUMMARY OF THE INVENTION

The present disclosure is directed to host bacterial strains, methods of making such host bacterial strains and methods of using such host bacterial strains to improve plasmid production.

In some embodiments, an engineered E. coli host cell is provided that has a knockout of SbcC, SbcD or both but without certain additional mutations.

In some embodiments, a method for preparing an engineered E. coli host cell of the present disclosure is provided.

In some embodiments, methods for replicating a vector in an engineered E. coli host cell of the present disclosure are provided.

### BRIEF DESCRIPTION OF THE DRAWINGS

For a more complete understanding of the present invention and the advantages thereof, reference is now made to the following description taken in conjunction with the accompanying drawings.

FIG. 1A depicts the pKD4 SbcCD targeting PCR fragment.

FIG. 1B depicts the SbcCD locus.

FIG. 1C depicts the integrated pKD4 PCR product knocking out SbcCD.

FIG. 1D depicts the scar after FRT-mediated excision of the pKD4 kanR marker.

### DETAILED DESCRIPTION OF THE INVENTION

The present disclosure provides bacterial host strains, methods for modifying bacterial host strains, and methods for manufacturing that can improve plasmid yield and quality.

The bacterial hosts strains and methods of the present disclosure can enable improved manufacturing of vectors such as non-viral transposon (transposase vector, Sleeping Beauty transposon vector, Sleeping Beauty transposase vector, PiggyBac transposon vector, PiggyBac transposase vector, expression vector, etc.) or Non-viral Gene Editing (e.g. Homology-Directed Repair (HDR)/CRISPR-Cas9) vectors for cell therapy, gene therapy or gene replacement applications, and viral vectors (e.g. AAV vector, AAV rep cap vector, AAV helper vector, Ad helper vector, Lentivirus vector, Lentiviral envelope vector, Lentiviral packaging vector, Retroviral vector, Retroviral envelope vector, Retroviral packaging vector, etc.) for cell therapy, gene therapy or gene replacement applications.

Improved plasmid manufacturing can include improved plasmid yield, improved plasmid stability (e.g., reduced plasmid deletion, inversion, or other recombination products) and/or improved plasmid quality (e.g., decreased nicked, linear or dimerized products) and/or improved plasmid supercoiling (e.g., decreased reduced supercoiling topological isoforms) compared to plasmid manufacturing using an alternative host strain known in the art. It is to be understood that all references cited herein are incorporated by reference in their entirety.

### Definitions

As used herein, the singular forms “a”, “an” and “the” include plural referents unless the context clearly dictates otherwise.

The use of the term “or” in the claims and the present disclosure is used to mean “and/or” unless explicitly indicated to refer to alternatives only or the alternatives are mutually exclusive.

Use of the term “about”, when used with a numerical value, is intended to include +/−10%. By way of example but not limitation, if a number of amino acids is identified as about 200, this would include 180 to 220 (plus or minus 10%).

As used herein, “AAV vector” refers to an adeno-associated virus vector or episomal viral vector. By way of example, but not limitation, “AAV vector” includes self-complementary adeno-associated virus vectors (scAAV) and single-stranded adeno-associated virus vectors (ssAAV).

As used herein, “amp” refers to ampicillin.

As used herein, “ampR” refers to an ampicillin resistance gene.

As used herein “bacterial region” refers to the region of a vector, such as a plasmid, required for prorogation and selection in a bacterial host.

As used herein “Cat^{R}” refers to a chloramphenicol resistance gene.

As used herein “ccc” or “CCC” means “covalently closed circular” unless used in the context of a nucleotide or amino acid sequence.

As used herein, “cI” means lambda repressor.

As used herein “cITs857” refers to the lambda repressor further incorporating a C to T (Ala to Thr) mutation that confers temperature sensitivity. cITs857 is a functional repressor at 28-30° C. but is mostly inactive at 37-42° C. Also called cI857 or cI857ts.

As used herein “cmv” or “CMV” refers to cytomegalovirus.

As used herein “copy cutter host strain” refers to R6K origin production strains containing a phage φ80 attachment site chromosomally integrated copy of an arabinose inducible CI857ts gene. Addition of arabinose to plates or media (e.g. to 0.2-0.4% final concentration) induces pARA mediated CI857ts repressor expression which reduces copy number at 30° C. through CI857ts mediated downregulation of the R6K Rep protein expressing pL promoter [i.e. additional CI857ts mediates more effective downregulation of the pL (OL1-G to T) promoter at 30° C.]. Copy number induction after temperature shift to 37-42° C. is not impaired since the CI857ts repressor is inactivated at these elevated temperatures. Copy cutter host strains increase the R6K vector temperature upshift copy number induction ratio by reducing the copy number at 30° C. This is advantageous for production of large, toxic, or dimerization prone R6K origin vectors.

As used herein “dcm methylation” refers to methylation by E. coli methyltransferase that methylates the sequences CC(A/T)GG at the C5 position of the second cytosine.

As used herein, “derived from” means that a cell has been descended from a particular cell line. For example, derived from DH5α means that the cell is made from DH5α or a descendant of DH5α. As such, the derivative cell can include polymorphisms and other changes that occur to the cell line as it is cultured.

As used herein “EGFP” refers to enhanced green fluorescent protein.

As used herein, “engineered E. coli strain” should be understood to refer to an E. coli strain of the present disclosure that has a gene knockout (or knockdown) in SbcC, SbcD or both that was made by human intervention.

As used herein, “engineered mutation” should be understood a mutation that did not naturally occur and was instead the product of direct, human intervention.

As used herein “eukaryotic expression vector” refers to a vector for expression of mRNA, protein antigens, protein therapeutics, shRNA, RNA or microRNA genes in a target eukaryotic organism using RNA Polymerase I, II or III promoters.

As used herein “eukaryotic region” refers to the region of a plasmid that encodes eukaryotic sequences and/or sequences required for plasmid function in the target organism. This includes the region of a plasmid vector required for expression of one or more transgenes in the target organism including RNA Pol II enhancers, promoters, transgenes and polyA sequences. This also includes the region of a plasmid vector required for expression of one or more transgenes in the target organism using RNA Pol I or RNA Pol III promoters, RNA Pol I or RNA Pol III expressed transgenes or RNAs. The eukaryotic region may optionally include other functional sequences, such as eukaryotic transcriptional terminators, supercoiling-induced DNA duplex destabilized (SIDD) structures, S/MARs, boundary elements, and the like. In a Lentiviral or Retroviral vector, the eukaryotic region contains flanking direct repeat LTRs, in a AAV vector the eukaryotic region contains flanking inverted terminal repeats, while in a Transposon vector the eukaryotic region contains flanking transposon inverted terminal repeats or IR/DR termini (e.g., Sleeping Beauty). In genome integration vectors, the eukaryotic region may encode homology arms to direct targeted integration.

As used herein “expression vector” refers to a vector for expression of mRNA, protein antigens, protein therapeutics, shRNA, RNA or microRNA genes in a target organism.

As used herein “gene of interest” refers to a gene to be expressed in the target organism. Includes mRNA genes that encode protein or peptide antigens, protein or peptide therapeutics, and mRNA, shRNA, RNA or microRNA that encode RNA therapeutics, and mRNA, shRNA, RNA or microRNA that encode RNA vaccines, and the like.

As used herein “genomic” as it relates to Rep proteins and promoters, RNA-IN, including RNA-IN regulated selectable markers, antibiotic resistance markers, and lambda repressors refers to nucleic acid sequences incorporated in the bacterial host strain.

As used herein “high yield plasmid manufacturing host” refers to recA-, endA- cell lines such as DH1, DH5α, JM107, JM108, JM109, MG1655 and XL1Blue that do not contain viability- or yield-reducing mutations in sbcB, recB, recD, and recJ and, optionally, uvrC, mcrA and/or mcrBC-hsd-mrr.

As used herein “HyperGRO fermentation process” refers to fed-batch fermentation, in which plasmid-containing E. coli cells are grown at a reduced temperature during part of the fed-batch phase, during which growth rate is restricted, followed by a temperature up-shift and continued growth at elevated temperature in order to accumulate plasmid; the temperature shift at restricted growth rate improved plasmid yield and purity.

As used herein “inverted repeat” refers to a single-stranded sequence of nucleotides followed downstream by its reverse complement. The intervening sequence of nucleotides between the initial sequence and the reverse complement can be any length including zero. When the intervening length is zero, the composite sequence is a palindrome. It should be understood that inverted repeats can occur in double-stranded DNA and that other inverted repeats can occur within the intervening sequence.

As used herein “IR/DR” refers to inverted repeats which are directly repeated twice. For example, Sleeping Beauty transposon IR/DR repeats.

As used herein “iteron” refers to directly repeated DNA sequences in a origin of replication that are required for replication initiation. R6K origin iteron repeats are 22 bp such as SEQ ID NOs 19-23 of WO 2019/183248 (aaacatgaga gcttagtacg tg, aaacatgaga gcttagtacg tt, agccatgaga gcttagtacg tt, agccatgagg gtttagttcg tt, and aaacatgaga gcttagtacg ta, respectively).

As used herein “ITR” refers to an inverted terminal repeat.

As used herein “kan” refers to kanamycin.

As used herein “kanR” refers to a kanamycin resistance gene.

As used herein, “knockdown” refers to disruption of a gene that results in a reduced expression of the gene product and/or reduced activity of the gene product.

As used herein, “knockout” refers to disruption of a gene which results in ablation of gene expression from the gene and/or the expressed gene product is non-functional.

As used herein “kozak sequence” refers to an optimized consensus DNA sequence gccRccATG (R=G or A) immediately upstream of an ATG start codon that ensures efficient tranlation initiation. A SalI site (GTCGAC) immediately upstream of the ATG start codon (GTCGACATG) is an effective kozak sequence.

As used herein “lentiviral vector” refers to an integrative viral vector that can infect dividing and non-dividing cells. Also called a Lentiviral transfer plasmid. The Plasmid encodes Lentiviral LTR flanked expression unit. Transfer plasmid is transfected into production cells along with Lentiviral envelope and packaging plasmids required to make viral particles.

As used herein “lentiviral envelope vector” refers to a plasmid encoding envelope glycoprotein.

As used herein “lentiviral packaging vector” refers to one or two plasmids that express gag, pol and Rev gene functions required to package the lentiviral transfer vector.

As used herein “minicircle” refers to covalently closed circular plasmid derivatives in which the bacterial region has been removed from the parent plasmid by in vivo or in vitro site-specific recombination or in vitro restriction digestion/ligation. Minicircle vectors are replication incompetent in bacterial cells.

As used herein “mSEAP” refers to murine secreted alkaline phosphatase.

As used herein “Nanoplasmid™ vector” refers to a vector combining an RNA selectable marker with a R6K, ColE2 or ColE2 related replication origin. For example, NTC9385C, NTC9685C, NTC9385R, NTC9685R vectors and modifications described in WO 2014/035457.

As used herein, “mutation” can refer to any type of mutation such as a substitution, addition, deletion.

As used herein, “non-functional” with respect to the SbcCD complex refers to a SbcCD complex that cannot cleave palindromic sequences.

As used herein “NTC8 series” refers to vectors, such as NTC8385, NTC8485 and NTC8685 plasmids are antibiotic-free pUC origin vectors that contain a short RNA (RNA-OUT) selectable marker instead of an antibiotic resistance marker such as kanR. The creation and application of these RNA-OUT based antibiotic-free vectors are described in WO2008/153733.

As used herein “NTC9385R” refers to the NTC9385R Nanoplasmid™ vector described in WO 2014/035457 and has a spacer region encoded NheI-trpA terminator-R6K origin RNA-OUT-KpnI bacterial region linked through the flanking NheI and KpnI sites to the eukaryotic region.

As used herein “OD₆₀₀” refers to optical density at 600 nm.

As used herein PCR refers to “polymerase chain reaction.”

As used herein “pDNA” refers to plasmid DNA.

As used herein “piggyback transposon” refers to a transposon system that integrates an ITR flanked PB transposon into the genome by a simple cut and paste mechanism mediated by PB transposase. The transposon vector typically contains a promoter-transgene-polyA expression cassette between the PB ITRs which is excised and integrated into the genome.

As used herein “pINT pR pL vector” refers to the pINT pR pL att_{HK022} integration expression vector is described in Luke et al., 2011 Mol Biotechnol 47:43 and included herein by reference. The target gene to be expressed is cloned downstream of the pL promoter. The vector encodes the temperature inducible cI857 repressor, allowing heat inducible target gene expression.

As used herein “P_{L} promoter” refers to the lambda promoter left. P_{L} is a strong promoter that is repressed by the cI repressor binding to OL1, OL2 and OL3 repressor binding sites. The temperature sensitive cI857 repressor allows control of gene expression by heat induction since at 30° C. the cI857 repressor is functional and it represses gene expression, but at 37-42° C. the repressor is inactivated so expression of the gene ensues.

As used herein “P_{L} (OL1 G to T) promoter” refers to the lambda promoter left with a OL1 G to T mutation. P_{L} is a strong promoter that is repressed by the cI repressor binding to OL1, OL2 and OL3 repressor binding sites. The temperature sensitive cI857 repressor allows control of gene expression by heat induction since at 30° C. the cI857 repressor is functional and it represses gene expression, but at 37-42° C. the repressor is inactivated so expression of the gene ensues. The cI repressor binding to OL1 is reduced by the OL1 G to T mutation resulting in increased promoter activity at 30° C. and 37-42° C. as described in WO 2014/035457.

As used herein “plasmid” refers to an extra chromosomal DNA molecule separate from the chromosomal DNA which is capable of replicating independently from the chromosomal DNA.

As used herein “plasmid copy number” refers to the number of copies of plasmid per cell. Increases in plasmid copy number indicate an increase in plasmid production yield.

As used herein “Pol” refers to polymerase.

As used herein “Pol I” refers to E. coli DNA Polymerase I.

As used herein “Pol III” refers to E. coli DNA Polymerase III.

As used herein “Pol III dependent origin of replication” refers to a replication origin that doesn't require Pol I, for example the rep protein dependent R6K gamma replication origin. Numerous additional Pol III dependent replication origins are known in the art, many of which are summarized in del Solar et al., Supra, 1998 which is included herein by reference.

As used herein “polyA” refers to a polyadenylation signal or site. Polyadenylation is the addition of a poly(A) tail to an RNA molecule. The polyadenylation signal contains the sequence motif recognized by the RNA cleavage complex. Most human polyadenylation signals contain an AAUAAA motif and conserved sequences 5′ and 3′ to it. Commonly utilized polyA signals are derived from the rabbit β globin, bovine growth hormone, SV40 early, or SV40 late polyA signals.

As used herein a “polyA repeat” refers to a consecutive sequence of adenine nucleotides as a direct repeat. Similarly, a “polyG repeat” refers to a consecutive sequence of guanine nucleotides as a direct repeat, a “polyC repeat” refers to a consecutive sequence of cytosine nucleotides as a direct repeat, and a “polyT repeat” refers to a consecutive sequence of thymine nucleotides as a direct repeat. A “mRNA vector” contains polyA repeats.

As used herein “pUC origin” refers to a pBR322-derived replication origin, with G to A transition that increases copy number at elevated temperature and deletion of the ROP negative regulator.

As used herein “pUC free” refers to a plasmid that does not contain the pUC origin.

As used herein “pUC plasmid” refers to a plasmid containing the pUC origin.

As used herein “R6K plasmid” refers to a plasmid with a R6K or R6K-derived origin of replication such as NTC9385R, NTC9685R, NTC9385R2-01, NTC9385R2-02, NTC9385R2a-O1, NTC9385R2a-O2, NTC9385R2b-O1, NTC9385R2b-02, NTC9385Ra-O1, NTC9385Ra-O2, NTC9385RaF, and NTC9385RbF vectors as well as modifications and alternative vectors containing a R6K replication origin that were described in WO 2014/035457 and WO2019/183248. Alternative R6K vectors known in the art including, but not limited to, pCOR vectors (Gencell), pCpGfree vectors (Invivogen), and CpG free University of Oxford vectors including pGM169.

As used herein “R6K replication origin” refers to a region which is specifically recognized by the R6K Rep protein to initiate DNA replication, including, but not limited to, R6K gamma replication origin sequence disclosed as SEQ ID NO:1, SEQ ID NO:2 SEQ ID NO:4, and SEQ ID NO:18 in WO 2019/183248 (SEQ ID NOs: 43-44, 46 and 60, respectively). Also included are CpG free versions (e.g. SEQ ID NO:3) as described in Drocourt et al., U.S. Pat. No. 7,244,609, which is incorporated herein by reference (SEQ ID NO: 63).

As used herein “R6K replication origin-RNA-OUT bacterial origin” contains a R6K replication origin for propagation and the RNA-OUT selectable marker (e.g. SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17 disclosed in WO 2019/183248 (SEQ ID NOs: 50-59, respectively).

As used herein “Rep protein dependent plasmid” refers to a plasmid in which replication is dependent on a replication (Rep) protein provided in Trans. For example, R6K replication origin, ColE2-P9 replication origin and ColE2 related replication origin plasmids in which the Rep protein is expressed from the host strain genome. Numerous additional Rep protein dependent plasmids are known in the art, many of which are summarized in del Solar et al., Supra, 1998, Microbiol. Mol. Biol. Rev. 62:44-464 which is incorporated herein by reference.

As used herein “retroviral vector” refers to integrative viral vector that can infect dividing cells. Also call transfer plasmid. Plasmid encodes Retroviral LTR flanked expression unit. Transfer plasmid is transfected into production cells along with envelope and packaging plasmids required to make viral particles.

As used herein “retroviral envelope vector” refers to a plasmid encoding envelope glycoprotein.

As used herein “retroviral packaging vector” refers to a plasmid that encodes retroviral gag and pol genes required to package the retroviral transfer vector.

As used herein “RNA-IN” refers to an insertion sequence 10 (IS10) encoded RNA-IN, an RNA complementary and antisense to a portion of RNA RNA-OUT. When RNA-IN is cloned in the untranslated leader of a mRNA, annealing of RNA-IN to RNA-OUT reduces translation of the gene encoded downstream of RNA-IN.

As used herein “RNA-IN regulated selectable marker” refers to a genomically expressed RNA-IN regulated selectable marker. In the presence of plasmid borne RNA-OUT antisense repressor RNA (e.g. SEQ ID NO: 6 disclosed in WO 2019/183248 (SEQ ID NO: 48)), expression of a protein encoded downstream of RNA-IN (e.g. having sequence gccaaaaatcaataatcagacaacaagatg) is repressed. An RNA-IN regulated selectable marker is configured such that RNA-IN regulates either 1) a protein that is lethal or toxic to said cell per se or by generating a toxic substance (e.g., SacB), or 2) a repressor protein that is lethal or toxic to said bacterial cell by repressing the transcription of a gene that is essential for growth of said cell (e.g. murA essential gene regulated by RNA-IN tetR repressor gene). For example, genomically expressed RNA-IN-SacB cell lines for RNA-OUT plasmid selection/propagation are described in WO 2008/153733. Alternative selection markers described in the art may be substituted for SacB.

As used herein “RNA-OUT” refers to an insertion sequence 10 (IS10) encoded RNA-OUT, an antisense RNA that hybridizes to, and reduces translation of, the transposon gene expressed downstream of RNA-IN. The sequence of the RNA-OUT RNA (SEQ ID NO: 6 disclosed in WO 2019/183248 (SEQ ID NO: 48)) and complementary RNA-IN SacB genomically expressed RNA-IN-SacB cell lines can be modified to incorporate alternative functional RNA-IN/RNA-OUT binding pairs such as those described in Mutalik et al., 2012 Nat Chem Biol 8:447, including, but not limited to, the RNA-OUT A08/RNA-IN S49 pair, the RNA-OUT A08/RNA-IN S08 pair, and CpG free modifications of RNA-OUT A08 that modify the CG in the RNA-OUT 5′ TTCGC sequence to a non-CpG sequence. A multitude of alternative substitutions to remove the two CpG motifs (mutating each CpG to either CpA, CpC, CpT, ApG, GpG, or TpG) may be utilized to make a CpG free RNA-OUT.

As used herein “RNA-OUT selectable marker” refers to an RNA-OUT selectable marker DNA fragment including E. coli transcription promoter and terminator sequences flanking an RNA-OUT RNA. An RNA-OUT selectable marker, utilizing the RNA-OUT promoter and terminator sequences, that is flanked by DraIII and KpnI restriction enzyme sites, and designer genomically expressed RNA-IN-SacB cell lines for RNA-OUT plasmid propagation, are described in WO 2008/153733 and included herein by reference. The RNA-OUT promoter and terminator sequences that flank the RNA-OUT RNA may be replaced with heterologous promoter and terminator sequences. For example, the RNA-OUT promoter may be substituted with a CpG free promoter known in the art, for example the I-EC2K promoter or the P5/6 5/6 or P5/6 6/6 promoters described in WO 2008/153733 and included herein by reference. A 2 CpG RNA-OUT selectable marker in which the two CpG motifs in the RNA-OUT promoter are removed was given as SEQ ID NO: 7 in WO 2019/183248 (SEQ ID NO: 49). Vectors incorporating CpG free RNA-OUT selectable marker may be selected for sucrose resistance using the RNA-IN-SacB cell lines for RNA-OUT plasmid propagation described in WO 2008/153733 or any cell line with RNA-IN-SacB as described in WO 2008/153733. Alternatively, the RNA-IN sequence in these cell lines can be modified to incorporate the 1 bp change needed to perfectly match the CpG free RNA-OUT region complementary to RNA-IN.

As used herein “RNA selectable marker” refers to a plasmid borne expressed non-translated RNA that regulates a chromosomally expressed target gene to afford selection. This may be a plasmid borne nonsense suppressing tRNA that regulates a nonsense suppressible selectable chromosomal target as described by Crouzet J and Soubrier F 2005 U.S. Pat. No. 6,977,174 included herein by reference. This may also be a plasmid borne antisense repressor RNA, a non limiting list included herein by reference includes RNA-OUT that represses RNA-IN regulated targets (WO 2008/153733), pMB1 plasmid origin encoded RNAI that represses RNAII regulated targets (Grabherr R, Pfaffenzeller I. 2006 US patent application US20060063232; Cranenburgh R M. 2009; U.S. Pat. No. 7,611,883), IncB plasmid pMU720 origin encoded RNAI that represses RNA II regulated targets (Wilson I W, Siemering K R, Praszkier J, Pittard A J. 1997. J Bacteriol 179:742-53), ParB locus Sok of plasmid R1 that represses Hok regulated targets, Flm locus FlmB of F plasmid that represses fimA regulated targets (Morsey M A, 1999 U.S. Pat. No. 5,922,583). An RNA selectable marker may be another natural antisense repressor RNAs known in the art such as those described in Wagner E G H, Altuvia S, Romby P. 2002. Adv Genet 46:361-98 and Franch T, and Gerdes K. 2000. Current Opin Microbiol 3:159-64. An RNA selectable marker may also be an engineered repressor RNAs such as synthetic small RNAs expressed SgrS, MicC or MicF scaffolds as described in Na D, Yoo S M, Chung H, Park H, Park J H, Lee S Y. 2013. Nat Biotechnol 31:170-4. An RNA selectable marker may also be an engineered repressor RNA as part of a selectable marker that represses a target RNA fused to a target gene to be regulated such as SacB as described in US 2015/0275221.

As used herein “SacB” refers to the structural gene encoding Bacillus subtilus levansucrase. Expression of SacB in gram negative bacteria is toxic in the presence of sucrose.

As used herein “SEAP” refers to secreted alkaline phosphatase.

As used herein “selectable marker” or “selection marker” refer to a selectable marker, for example, a kanamycin resistance gene or a RNA selectable marker.

As used herein, the term “sequence identity” refers to the degree of identity between any given query sequence and a subject sequence. A subject sequence may, for example, have at least 90%, at least 95%, at least 98%, at least 99% or 100% sequence identity to a given query sequence. To determine percent sequence identity, a query sequence (e.g. a nucleic acid sequence) is aligned to one or more subject sequences using any suitable sequence alignment program that is well known in the art, for instance, the computer program ClustalW (version 1.83, default parameters), which allows alignments of nucleic acid sequences to be carried out across their entire length (global alignment). Chema et al., 2003 Nucleic Acids Res., 31:3497-500. In a preferred method, the sequence alignment program (e.g. ClustalW) calculates the best match between a query and one or more subject sequences, and aligns them so that identities, similarities, and differences can be determined. Gaps of one or more nucleotides can be inserted into a query sequence, a subject sequence, or both, to maximize sequence alignments. For fast pair-wise alignments of nucleic acid sequences, suitable default parameters can be selected that are appropriate for the particular alignment program. The output is a sequence alignment that reflects the relationship between sequences. To further determine percent identity of a subject nucleic acid sequence to a query sequence, the sequences are aligned using the alignment program, the number of identical matches in the alignment is divided by the length of the query sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

As used herein “shRNA” refers to short hairpin RNA.

As used herein “S/MAR” refers to scaffold/matrix attached region which includes eukaryotic sequences that mediate DNA attachment to the nuclear matrix.

As used herein “Sleeping Beauty Transposon” refers to a transposon system that integrates an IR/DR flanked SB transposon into the genome by a simple cut and paste mechanism mediated by SB transposase. The transposon vector typically contains a promoter-transgene-polyA expression cassette between the IR/DRs which is excised and integrated into the genome.

As used herein “spacer region” refers to the region linking the 5′ and 3′ ends of the eukaryotic region sequences. The eukaryotic region 5′ and 3′ ends are typically separated by the bacterial replication origin and bacterial selectable marker in plasmid vectors (bacterial region) so many spacer regions consist of the bacterial region. In Pol III dependent origin of replication vectors of the invention, this spacer region preferably is less than 1000 bp.

As used herein “structured DNA sequence” refers to a DNA sequence that is capable of forming replication inhibiting secondary structures (Mirkin and Mirkin, 2007. Microbiology and Molecular Biology Reviews 71:13-35). This includes but is not limited to inverted repeats, palindromes, direct repeats, IR/DRs, homopolymeric repeats or repeat containing eukaryotic promoter enhancers, or repeat containing eukaryotic origin of replications.

As used herein “SV40 origin” refers to Simian Virus 40 genomic DNA that contains the origin of replication.

As used herein “SV40 enhancer” refers to Simian Virus 40 genomic DNA that contains the 72 bp and optionally the 21 bp enhancer repeats.

As used herein “TE Buffer” refers to a solution containing approximately 10 mM Tris pH 8 and 1 mM EDTA.

As used herein “TetR” refers to a tetracycline resistance gene.

As used herein “transcription terminator” refers to (1) in the bacterial context, a DNA sequence that marks the end of a gene or operon for transcription. This may be an intrinsic transcription terminator or a Rho-dependent transcriptional terminator. For an intrinsic terminator, such as the trpA terminator, a hairpin structure forms within the transcript that disrupts the mRNA-DNA-RNA polymerase ternary complex. Alternatively. Rho-dependent transcriptional terminators require Rho factor, an RNA helicase protein complex, to disrupt the nascent mRNA-DNA-RNA polymerase ternary complex; or (2) in the eukaryotic context, PolyA signals are not ‘terminators’, instead internal cleavage at PolyA sites leaves an uncapped 5′end on the 3′UTR RNA for nuclease digestion. Nuclease catches up to RNA Pol II and causes termination. Termination can be promoted within a short region of the poly A site by introduction of RNA Pol II pause sites (eukaryotic transcription terminator). Pausing of RNA Pol II allows the nuclease introduced into the 3′ UTR mRNA after PolyA cleavage to catch up to RNA Pol II at the pause site. A nonlimiting list of eukaryotic transcription terminators know in the art include the C2×4 and the gastrin terminator. Eukaryotic transcription terminators may elevate mRNA levels by enhancing proper 3′-end processing of mRNA.

As used herein “transfection” refers to a method to deliver nucleic acids into cells [e.g. poly(lactide-co-glycolide) (PLGA), ISCOMs, liposomes, niosomes, virosomes, block copolymers, Pluronic block copolymers, chitosan, and other biodegradable polymers, microparticles, microspheres, calcium phosphate nanoparticles, nanoparticles, nanocapsules, nanospheres, poloxamine nanospheres, electroporation, nucleofection, piezoelectric permeabilization, sonoporation, iontophoresis, ultrasound, SQZ high speed cell deformation mediated membrane disruption, corona plasma, plasma facilitated delivery, tissue tolerable plasma, laser microporation, shock wave energy, magnetic fields, contactless magneto-permeabilization, gene gun, microneedles, microdermabrasion, hydrodynamic delivery, high pressure tail vein injection, etc] as known in the art and included herein by reference. Transfection of DNA into E. coli, commonly called transformation, is typically performed using chemical competent E. coli or electrocompetent E. coli cells using standard methodologies as known in the art and included herein by reference.

As used herein “transgene” refers to a gene of interest that is cloned into a vector for expression in a target organism.

As used herein “transposase vector” refers to a vector which encodes a transposase.

As used herein “transposon vector” refers to a vector which encodes a transposon which is a substrate for transposase-mediated gene integration.

As used herein “ts” means temperature-sensitive.

As used herein “UTR” refers to an untranslated region of mRNA (5′ or 3′ to the coding region).

As used herein “vector” refers to a gene delivery vehicle, including viral (e.g. Alphavirus, Poxvirus, Lentivirus, Retrovirus, Adenovirus, Adenovirus related virus, etc.) and non-viral (e.g. plasmid, MIDGE, transcriptionally active PCR fragment, minicircles, bacteriophage, Nanoplasmid™, etc.) vectors. These are well known in the art and are included herein by reference.

As used herein “vector backbone” refers to the eukaryotic and bacterial region of a vector, without the transgene or target antigen coding region.

In some embodiments, an engineered Escherichia coli (E. coli) host cell, wherein the engineered E. coli host cell comprises a gene knockout of at least one gene selected from the group consisting of SbcC and SbcD, and wherein the engineered E. coli host cell does not include an engineered viability- or yield-reducing mutation in any of sbcB, recB, recD, and recJ and, optionally, at least one of uvrC, mcrA, mcrBC-hsd-mrr and combinations thereof. In some embodiments, the engineered E. coli host cell does not include any engineered mutations in any of sbcB, recB, recD, and recJ and, optionally, at least one of uvrC, mcrA, mcrBC-hsd-mrr and combinations thereof. In some embodiments, the engineered E. coli host cell does not include any mutations in any of sbcB, recB, recD, and recJ and, optionally, at least one of uvrC, mcrA, mcrBC-hsd-mrr and combinations thereof.

It should be understood that, within the scope of the present disclosure are engineered E. coli host cells comprising a gene knockout (or knockdown) of at least one gene selected from the group consisting of SbcC and SbcD, where the engineered E. coli host cells do not include an engineered viability- or yield-reducing mutation, or in some embodiments an engineered mutation or any mutation, in at least one of sbcB, recB, recD, recJ, uvrC, mcrA and mcrBC-hsd-mrr. It should also be understood that, within the scope of the present disclosure are engineered E. coli host cells comprising a gene knockout of at least one gene selected from the group consisting of SbcC and SbcD, where the engineered E. coli host cells do not include an engineered viability- or yield-reducing mutation, or in some embodiments an engineered mutation or any mutation, in at least one of sbcB, recB, recD, and recJ. In some embodiments, an engineered E. coli host cell comprises a gene knockout of at least one gene selected from the group consisting of SbcC and SbcD, but does not include a viability- or yield-reducing mutation, or in some embodiments an engineered or any mutation, in mcrA. In some embodiments, an engineered E. coli host cell comprises a gene knockout of at least one gene selected from the group consisting of SbcC and SbcD, wherein the engineered E. coli host cell does not include an engineered viability- or yield-reducing mutation, or in some other embodiments an engineered or any mutation, in any of sbcB, recB, recD, and recJ.

In other embodiments, the engineered E. coli host cell comprises a gene knockout of at least one gene selected from the group consisting of SbcC and SbcD, and does not include any engineered viability- or yield-reducing mutations in at least one of sbcB, recB, recD, recJ, uvrC, mcrA and mcrBC-hsd-mrr. In other embodiments, the engineered E. coli host cell comprises a gene knockout of at least one gene selected from the group consisting of SbcC and SbcD, and does not include any engineered mutations in at least one of sbcB, recB, recD, recJ, uvrC, mcrA and mcrBC-hsd-mrr. In other embodiments, the engineered E. coli host cell comprises a gene knockout of at least one gene selected from the group consisting of SbcC and SbcD, and does not include any mutations in at least one of sbcB, recB, recD, recJ, uvrC, mcrA and mcrBC-hsd-mrr. In some embodiments, the engineered E. coli host cell comprises a gene knockout of at least one gene selected from the group consisting of SbcC and SbcD, and does not include any mutations in sbcB, recB, recD, recJ and uvrC. In some embodiments, the engineered E. coli host cell comprises a gene knockout of at least one gene selected from the group consisting of SbcC and SbcD, and does not include any mutation in mcrA.

In some embodiments, an engineered E. coli host cell is provided that includes a gene knockout of at least on gene selected from the group consisting of SbcC and SbcD, where the engineered E. coli host cell does not include an engineered viability- or yield-reducing mutation in any of sbcB, recB, recD, and recJ. In any of the foregoing embodiments, the engineered E. coli host cell can not include any engineered mutations in sbcB, recB, recD, and recJ. In any of the foregoing embodiments, the engineered E. coli host cell can not include any mutations in any of sbcB, recB, recD, and recJ. In some embodiments, an engineered E. coli host cell is provided that includes a gene knockout of at least one gene selected from the group consisting of SbC and SbcD and the E. coli host cell is isogenic to the strain from which it is derived, the strain from which it is derived being selected from the group consisting of DH5α, DH1, JM107, JM108, JM109, MG1655 and XL1Blue. In some embodiments, an engineered E. coli host cell is provided that includes a gene knockout of at least one gene selected from the group consisting of SbC and SbcD and the E. coli host cell is isogenic to the strain from which it is derived, the strain from which it is derived being selected from the group consisting of DH5α(dcm−), NTC4862, NTC4862-HF, NTC1050811, NTC1050811-HF, NTC1050811-HF (dcm−), HB101, TG1, and NEB Turbo.

To the extent not inconsistent with any of the foregoing embodiments, the engineered E. coli host cell can further not include an engineered viability- or yield-reducing mutation in at least one of uvrC, mcrA, mcrBC-hsd-mrr, and combinations thereof. In any of the foregoing embodiments, the engineered E. coli host cell can further not include any engineered mutations in at least one of uvrC, mcrA, mrBC-hsd-mrr, and combinations thereof. In any of the foregoing embodiments, the engineered E. coli host cell can further not include any mutations in at least one of uvrC, mcrA, mrBC-hsd-mrr, and combinations thereof. Thus, in some embodiments, the engineered E. coli host cell further does not include an engineered viability- or yield-reducing mutation, engineered mutation, or any mutation in uvrC. In other embodiments, the engineered E. coli host cell further does not include an engineered viability- or yield-reducing mutation, engineered mutation, or any mutation in mcrA. In still other embodiments, the engineered E. coli host cell further does not include an engineered viability- or yield-reducing mutation, engineered mutation, or any mutation in mcrBC-hsd-mrr. In yet other embodiment, the engineered E. coli host cell further does not include an engineered viability- or yield-reducing mutation, engineered mutation, or any mutation in mcrA and mrBC-hsd-mur. It should be understood that throughout this disclosure mrBC-hsd-mrr refers to a sequence that includes the sequences of SEQ ID NOs: 16-21.

In any of the foregoing embodiments, the engineered E. coli host cell can include a non-functional SbcCD complex or, in other words, can not include a functional SbcCD complex. Alternatively, in some embodiments, the engineered E. coli host cell can not include a SbcCD complex.

In any of the foregoing embodiments, the gene knockout of the engineered E. coli host cell can be a knockout of SbcC. Alternatively, in some embodiments, the gene knockout of the engineered E. coli host cell can be a knockout of SbcD. In any of the foregoing embodiments, the gene knockout of the engineered E. coli host cell can be a knockout of both SbcC and SbcD.

In any of the foregoing embodiments, the engineered E. coli host cell can be derived from a cell line selected from the group consisting of DH5α, DH1, JM107, JM108, JM109, MG1655 and XL1Blue. In any of the foregoing embodiments, the engineered E. coli host cell can be derived from DH5α (dcm−), NTC4862, NTC4862-HF, NTC1050811, NTC1050811-HF, or NTC1050811-HF (dcm-). In some of the foregoing embodiments, the engineered E. coli host cell can be derived from a cell line selected from the group consisting of HB101, TG1, and NEB Turbo. The genotypes for these cells lines are as follows:
DH5α (dcm−): DH5α dcm−
NTC4862: DH5α att_{λ}::P_{c}-RNA-IN-SacB, catR
NTC4862-HF: DH5α att_{λ}::P_{c}-RNA-IN-SacB, catR; att_{φ80}::pARA-CI857ts P_{c}-RNA-IN-SacB, tetR
NTC1050811: DH5α att_{λ}::P_{c}-RNA-IN-SacB, catR; att_{HK022}::pL (OL1-G to T) P42L-P106I-F107S P113S (P3−), SpecR StrepR; att_{φλ}::pARA-CI857ts, tetR
NTC1050811-HF: DH5α att_{λ}::P_{c}-RNA-IN− SacB, catR; att_{HK022}::pL (OL1-G to T) P42L-P106I-F107S P113S (P3−), SpecR StrepR; att_{φλ}::pARA-CI857ts P_{c}-RNA-IN-SacB, tetR
NTC1050811-HF (dcm−): DH5α dcm−att_{λ}::P_{c}-RNA-IN− SacB, catR; att_{HK022}::pL (OL1-G to T) P42L-P106I-F107S P113S (P3−), SpecR StrepR; att_{φ80}::pARA-CI857ts P_{c}-RNA-IN-SacB, tetR
HB101: F⁻ mcrB mrr hsdS20(r_{B}⁻ m_{B}⁻) recA13 leuB6 ara-14 proA2 lacY1 galK2 xyl-5 mtl-1 rpsL20(Sm^{R}) glnV44λ⁻
TG1: K-12 ginV44 thi-1 Δ(lac-proAB) Δ(mcrB-hsdSM)5(r_{K} ⁻m_{K}⁻) F′ [traD36 proAB⁺ lacI^{q} lacZΔM15]
NEB Turbo: FproA⁺B⁺ lacI^{q} ΔlacZM1/fhuA2 Δ(lac-proAB) glnV galK16 galE15 R(zgb-210::Tn10)Tets endA1 thi-1 Δ(hsdS-mcrB)5

In any of the foregoing embodiments, the engineered E. coli host cell can further include a genomic antibiotic resistance marker. By way of example, but not limitation, the genomic antibiotic resistance marker can be kanR comprising a sequence having at least 90%, at least 95%, at least 95%, at least 98%, at least 99% or 100% sequence identity to SEQ ID NO: 23 (kanR, 795 bp). By way of further example, but not limitation, the genomic antibiotic resistance marker can be kanR comprising a sequence encoding a protein having at least 90%, at least 95%, at least 98%, at least 99% or 100% sequence identity to SEQ ID NO: 36 (kanR). By way of still further example, the genomic antibiotic resistance marker can be a chloramphenicol resistance marker, gentamicin resistance marker, kanamycin resistance marker, spectinomycin and streptomycin resistance marker, trimethoprim resistance marker, or a tetracycline resistance marker. Alternatively, in any of the foregoing embodiments, the E. coli host cell can not include a genomic antibiotic resistance marker.

In any of the foregoing embodiments, the engineered E. coli host cell can further include a Rep protein suitable for culturing a Rep protein dependent plasmid. By way of example, but not limitation, the engineered E. coli host cell can include a genomic nucleic acid sequence having at least 90%, at least 95%, at least 98%, at least 99% or 100% sequence identity to a sequence selected from the group consisting of SEQ ID NO: 26 (P42L-P106I-F107S-P113S, 918 bp), SEQ ID NO: 27 (P42L-Δ106-107-P113S, 912 bp), SEQ ID NO: 28 (P42L-P106L-F107S, 918 bp), and SEQ ID NO: 29 (P42L-P113S, 918 bp). By way of further example, but not limitation, the engineered E. coli host cell can include a genomic nucleic acid sequence encoding a Rep protein having at least 90%, at least 95%, at least 98%, at least 99% or 100% identity to an amino acid sequence selected from the group consisting of SEQ ID NO: 39 (P42L-P106I-F107S-P113S), SEQ ID NO: 40 (P42L-Δ106-107-P113S), SEQ ID NO: 42 (P42L-P106L-F107S), SEQ ID NO: 41 (P42L-P113S), SEQ ID NO: 34 (ColE2 wild-type), SEQ ID NO: 35 (ColE2 mutant G194D). By way of still further example, but not limitation, the engineered E. coli host cell can include a Rep protein having at least 90%, at least 95%, at least 98%, at least 99% or 100% identity to an amino acid sequence selected from the group consisting of SEQ ID NO: 39 (P42L-P106I-F107S-P113S), SEQ ID NO: 40 (P42L-Δ106-107-P113S), SEQ ID NO: 42 (P42L-P106L-F107S, 305aa), SEQ ID NO: 41 (P42L-P113S, 305aa), SEQ ID NO: 34 (ColE2 wild-type), SEQ ID NO: 35 (ColE2 mutant G194D). It should be understood that the nucleic acid sequences encoding the Rep protein in any of the foregoing embodiments can be under the control of a P_{L} promoter and that such P_{L} promoter can enable temperature-sensitive expression of the Rep protein if there is a lambda repressor present in the genome, such as cITs857. By way of example, but not limitation, the P_{L} promoter can have a sequence having at least 95%, at least 98%, at least 99% or 100% sequence identity to ttgacataaa taccactggc ggtgatact (P_{L} promoter (−35 to −10)), ttgacataaa taccactggc gtgatact (P_{L}promoter OL1-G (−35 to −10)), or ttgacataaa taccactggc gttgatact (P_{L} promoter OL1-G to T (−35 to −10)). It should be further understood that where the Rep protein is a R6K Rep protein such as SEQ ID NOs: 39-42, a vector that is transfected into the engineered E. coli host cell can contain a R6K origin of replication and, alternatively, where the Rep protein is a ColE2 Rep protein, a vector that is transfected into the engineered E. coli host cell can contain a ColE2 origin of replication.

In any of the foregoing embodiments, the engineered E. coli host cell can further include a genomic nucleic acid sequence encoding a genomically expressed RNA-IN regulated selectable marker. By way of example, but not limitation, the engineered E. coli host cell can include a genomic nucleic acid sequence (which encodes the selectable marker) that has at least 90%, at least 95%, at least 98%, at least 99% or 100% sequence identity to SEQ ID NO: 25 (SacB, 1422 bp). By way of further example, but not limitation, the engineered E. coli host cell can include a genomic nucleic acid sequence that encodes the selectable marker which has an amino acid sequence having at least 90%, at least 95%, at least 98%, at least 99% or 100% sequence identity to SEQ ID NO: 38 (SacB). By way of still further example, but not limitation, the engineered E. coli host cell can include a RNA-IN regulated selectable marker having an amino acid sequence having at least 90%, at least 95%, at least 98%, at least 99% or 100% sequence identity to SEQ ID NO: 38 (SacB). In any of the foregoing embodiments, the RNA-IN regulated selectable marker can be downstream of an RNA-IN having the sequence gccaaaaatcaataatcagacaacaagatg (SEQ ID NO: 66); in embodiments where this RNA-IN is used, the corresponding RNA-OUT in a vector can be that of SEQ ID NO: 6 of WO 2019/183248 (SEQ ID NO: 48). Thus, for SacB, the RNA-IN SacB sequence can be

(SEQ ID NO: 67)
gccaaaaatcaataatcagacaacaagatgaacatcaaaaagtttgcaaaacaagcaacagtattaacctttactaccgcactgctggca
ggaggcgcaactcaagcgtttgcgaaagaaacgaaccaaaagccatataaggaaacatacggcatttcccatattacacgccatgatat
gctgcaaatccctgaacagcaaaaaaatgaaaaatatcaagttcctgaattcgattcgtccacaattaaaaatatctcttctgcaaaaggcct
ggacgtttgggacagctggccattacaaaacgctgacggcactgtcgcaaactatcacggctaccacatcgtctttgcattagccggaga
tcctaaaaatgcggatgacacatcgatttacatgttctatcaaaaagtcggcgaaacttctattgacagctggaaaaacgctggccgcgtct
ttaaagacagcgacaaattcgatgcaaatgattctatcctaaaagaccaaacacaagaatggtcaggttcagccacatttacatctgacgg
aaaaatccgtttattctacactgatttctccggtaaacattacggcaaacaaacactgacaactgcacaagttaacgtatcagcatcagaca
gctctttgaacatcaacggtgtagaggattataaatcaatctttgacggtgacggaaaaacgtatcaaaatgtacagcagttcatcgatgaa
ggcaactacagctcaggcgacaaccatacgctgagagatcctcactacgtagaagataaaggccacaaatacttagtatttgaagcaaa
cactggaactgaagatggctaccaaggcgaagaatctttatttaacaaagcatactatggcaaaagcacatcattcttccgtcaagaaagt
caaaaacttctgcaaagcgataaaaaacgcacggctgagttagcaaacggcgctctcggtatgattgagctaaacgatgattacacactg
aaaaaagtgatgaaaccgctgattgcatctaacacagtaacagatgaaattgaacgcgcgaacgtctttaaaatgaacggcaaatggtac
ctgttcactgactcccgcggatcaaaaatgacgattgacggcattacgtctaacgatatttacatgcttggttatgtttctaattctttaact
ggcccatacaagccgctgaacaaaactggccttgtgttaaaaatggatcttgatcctaacgatgtaacctttacttactcacacttcgctgta
cctcaagcgaaaggaaacaatgtcgtgattacaagctatatgacaaacagaggattctacgcagacaaacaatcaacgtttgcgccaagcttc
ctgctgaacatcaaaggcaagaaaacatctgttgtcaaagacagcatccttgaacaaggacaattaacagttaacaaataa

It should be understood that any suitable RNA-IN regulated selected marker and RNA-IN can be used and these are known in the art.

In any of the foregoing embodiments, the engineered E. coli host cell can further include a genomic nucleic acid sequence encoding a temperature-sensitive lambda repressor.

By way of example, but not limitation, the temperature-sensitive lambda repressor can be cITs857. By way of example, but not limitation, the engineered E. coli host cell can include a genomic nucleic acid sequence (which encodes the temperature-sensitive lambda repressor) that has at least 90%, at least 95%, at least 98%, at least 99% or 100% sequence identity to SEQ ID NO: 24 (cITs857, 714 bp). By way of further example, but not limitation, the engineered E. coli host cell can further include a genomic nucleic acid sequence encoding cITs857 having an amino acid sequence with at least 90%, at least 95%, at least 98%, at least 99% or 100% sequence identity to SEQ ID NO: 37 (cITs857). By way of still further example, but not limitation, the engineered E. coli host cell can further include a temperature-sensitive lambda repressor having an amino acid sequence with at least 90%, at least 95%, at least 98%, at least 99% or 100% sequence identity to SEQ ID NO: 37 (cITs857). In any of the foregoing embodiments, where the engineered E. coli host cell further includes a genomic nucleic acid sequence encoding a temperature-sensitive lambda repressor, the temperature-sensitive lambda repressor can be a phage φ80 attachment site chromosomally integrated copy of an arabinose inducible CITs857 gene. By way of example, but not limitation, the cITs857 gene can be under the control of the pBAD promoter to provide arabinose inducibility (pBAD promoter,

SEQ ID NO: 68)
ctgcataatgtgcctgtcaaatggacgaagcagggattctgcaaaccctatgctactccgtcaagccgtcaattgtctgattcgttaccaatt
atgacaacttgacggctacatcattcactttttcttcacaaccggcacggaactcgctcgggctggccccggtgcattttttaaatacccgcg
agaaatagagttgatcgtcaaaaccaacattgcgaccgacggtggcgataggcatccgggtggtgctcaaaagcagcttcgcctggctg
atacgttggtcctcgcgccagcttaagacgctaatccctaactgctggcggaaaagatgtgacagacgcgacggcgacaagcaaacat
gctgtgcgacgctggcgatatcaaaattgctgtctgccaggtgatcgctgatgtactgacaagcctcgcgtacccgattatccatcggtgg
atggagcgactcgttaatcgcttccatgcgccgcagtaacaattgctcaagcagatttatcgccagcagctccgaatagcgcccttcccctt
gcccggcgttaatgatttgcccaaacaggtcgctgaaatgcggctggtgcgcttcatccgggcgaaagaaccccgtattggcaaatattg
acggccagttaagccattcatgccagtaggcgcgcggacgaaagtaaacccactggtgataccattcgcgagcctccggatgacgacc
gtagtgatgaatctctcctggcgggaacagcaaaatatcacccggtcggcaaacaaattctcgtccctgatttttcaccaccccctgaccg
cgaatggtgagattgagaatataacctttcattcccagcggtcggtcgataaaaaaatcgagataaccgttggcctcaatcggcgttaaac
ccgccaccagatgggcattaaacgagtatcccggcagcaggggatcattttgcgcttcagccatacttttcatactcccgccattcagaga
agaaaccaattgtccatattgcatcagacattgccgtcactgcgtcttttactggctcttctcgctaaccaaaccggtaaccccgcttattaaa
agcattctgtaacaaagcgggaccaaagccatgacaaaaacgcgtaacaaaagtgtctataatcacggcagaaaagtccacattgattat
ttgcacggcgtcacactttgctatgccatagcatttttatccataagattagcggatcctacctgacgctttttatcgcaactctctactgttt
ctccatacccgtttttttggctcgactagaaataattttgtttaactttaagaaggagatataacc,.

In some embodiments, an engineered E. coli host cell is provided having the following genotype: F− φ80lacZΔM15 Δ(lacZYA-argF) U169 recA1 endA1 hsdR17 (rₖ−, mₖ+) gal-phoA supE44λ-thi-1 gyrA96 relA1 ΔSbcDC::kanR.

In some embodiments, an engineered E. coli host cell is provided having the following genotype: F− φ80lacZΔM15 Δ(lacZYA-argF) U169 recA1 endA1 hsdR17 (rₖ−, mₖ+) gal-phoA supE44λ-thi-1 gyrA96 relA1 ΔSbcDC.

In some embodiments, an engineered E. coli host cell is provided having the following genotype: DH5α att_{HK022}::pL (OL1-G to T) P42L-P106I-F107S P113S (P3−), SpecR StrepR; ΔSbcDC::kanR.

In some embodiments, an engineered E. coli host cell is provided having the following genotype: DH5α att_{HK022}::pL (OL1-G to T) P42L-P106I-F107S P113S (P3−), SpecR StrepR; ΔSbcDC.

In some embodiments, an engineered E. coli host cell is provided having the following genotype: F− φ80lacZΔM15 Δ(lacZYA-argF) U169 recA1 endA1 hsdR17 (rₖ−, mₖ+) gal-phoA supE44λ-thi-1 gyrA96 relA1; ΔSbcDC::kanR.

In some embodiments, an engineered E. coli host cell is provided having the following genotype: DH5α dcm−; ΔSbcDC.

In some embodiments, an engineered E. coli host cell is provided having the following genotype: DH5α dcm−; ΔSbcDC::kanR.

In some embodiments, an engineered E. coli host cell is provided having the following genotype: DH5α att_{λ}::P_{c}-RNA-IN-SacB, catR; ΔSbcDC.

In some embodiments, an engineered E. coli host cell is provided having the following genotype: DH5α att_{λ}::P_{c}-RNA-IN-SacB, catR; ΔSbcDC::kanR.

In some embodiments, an engineered E. coli host cell is provided having the following genotype: DH5α att_{λ}::P_{c}-RNA-IN-SacB, catR; att_{φλ}::pARA-CI857ts P_{c}-RNA-IN-SacB, tetR; ΔSbcDC.

In some embodiments, an engineered E. coli host cell is provided having the following genotype: DH5α att_{λ}::P_{c}-RNA-IN-SacB, catR; att_{λ80}::pARA-CI857ts P_{c}-RNA-IN-SacB, tetR; ΔSbcDC::kanR.

In some embodiments, an engineered E. coli host cell is provided having the following genotype: DH5α att_{λ}::P_{c}-RNA-IN-SacB, catR; att_{HK022}::pL (OL1-G to T) P42L-P106I-F107S P113S (P3−), SpecR StrepR; att_{φ80}::pARA-CI857ts, tetR; ΔSbcDC.

In some embodiments, an engineered E. coli host cell is provided having the following genotype: DH5α att_{λ}::P_{c}-RNA-IN-SacB, catR; att_{HK022}::pL (OL1-G to T) P42L-P106I-F107S P113S (P3−), SpecR StrepR; att_{φ80}::pARA-CI857ts, tetR; ΔSbcDC::kanR.

In some embodiments, an engineered E. coli host cell is provided having the following genotype: DH5α att_{λ}::P_{c}-RNA-IN− SacB, catR; att_{HK022}::pL (OL1-G to T) P42L-P106I-F107S P113S (P3−), SpecR StrepR; att_{φ80}::pARA-CI857ts P_{c}-RNA-IN- SacB, tetR; ΔSbcDC.

In some embodiments, an engineered E. coli host cell is provided having the following genotype: DH5α att_{λ}::P_{c}-RNA-IN- SacB, catR; att_{HK022}::pL (OL1-G to T) P42 L-P106I-F107S P113S (P3−), SpecR StrepR; att_{φ80}::pARA-CI857ts P_{c}-RNA-IN− SacB, tetR; ΔSbcDC::kanR.

In some embodiments, an engineered E. coli host cell is provided having the following genotype: DH5α dcm-att_{λ}::P_{c}-RNA-IN-SacB, catR; att_{HK022}::pL (OL1-G to T) P42L-P106I-F107S P113S (P3−), SpecR StrepR; att_{φ80}::pARA-CI857ts P_{c}-RNA-IN- SacB, tetR; ΔSbcDC.

In some embodiments, an engineered E. coli host cell is provided having the following genotype: DH5α dcm-att_{λ}::P_{c}-RNA-IN- SacB, catR; att_{HK022}::pL (OL1-G to T) P42L-P106I-F107S P113S (P3−), SpecR StrepR; att_{φ80}::pARA-CI857ts P_{c}-RNA-IN- SacB, tetR; ΔSbcDC::kanR.

In any of the foregoing embodiments, the SbcC gene can include a sequence having at least 90%, at least 95%, at least 98%, at least 99% or 100% sequence identity to SEQ ID NO: 9. In any of the foregoing embodiments, the SbcD gene can include a sequence having at least 90%, at least 95%, at least 98%, at least 99% or 100% sequence identity to SEQ ID NO: 10. It should be understood that this can apply to the gene prior to knockout or knockdown or after, i.e. in the engineered E. coli host cell. For reference, a wild-type sequence of SbcC from NCBI (Reference Sequence: WP_206061808.1) for E. coli K12 is given by

Mkilslrlknlnslkgewkidftrepfasnglfaitgptgagkttlldaiclalyhetprlsnvsqsqndlmtrdtaeclaevefevkgea
yrafwsqnrarnqpdgnlqvprvelarcadgkiladkvkdkleltatltgldygrftrsmllsqgqfaaflnakpkeraelleeltgteiy
gqisamvfeqhksarteleklqaqasgvtlltpeqvqsltaslqvltdeekqlitaqqqeqqslnwltrqdelqqeasrrqqalqqalae
eekaqpqlaalslaqparnlrphweriaehsaalahirqqieevntrlqstmalrasirhhaakqsaelqqqqqslntwlqehdrfrqw
nnepagwraqfsqqtsdrehlrqwqqqlthaeqklnalaaitltltadevatalaqhaeqrplrqhlvalhgqivpqqkrlaqlqvaiq
nvtqeqtqrnaalnemrqrykektqqladvkticeqeariktleaqraqlqagqpcplcgstshpaveayqalepgvnqsrllalene
vkklgeegatlrgqldaitkqlqrdeneaqslrqdeqaltqqwqavtaslnitlqplddiqpwldaqdeherqlrllsqrhelqgqiaah
nqqiiqyqqqieqrqqlllttltgyaltlpqedeeeswlatrqqeaqswqqrqneltalqnriqqltpiletlpqsdelphceetvvlenw
rqvheqclalhsqqqtlqqqdvlaaqslqkaqaqfdtalqasvfddqqaflaalmdeqtltqleqlkqnlenqrrqaqtlvtqtaetlaq
hqqhrpddglaltvtveqiqqelaqthqklrenttsqgeirqqlkqdadnrqqqqtlmqqiaqmtqqvedwgylnsligskegdkfr
kfaqgltldnlvhlanqqltrlhgryllqrkasealevevvdtwqadavrdtrtlsggesflvslalalalsdlvshktridslfldegfgtld
setldtaldaldalnasgktigvishveamkeripvqikvkkinglgysklestfavk,

while a wild-type sequence of SbcD from GenBank (AAB18122.1) for E. coli K12 is given by Mlfrqgtvmrilhtsdwhlgqnfysksreaehqafldwlletaqthqvdaiivagdvfdtgsppsyartlynrfvvnlqqtgchlvvl agnhdsvatlnesrdimaflnttvvasaghapqilprrdgtpgavlcpipflrprdiitsqaglngiekqqhllaaitdyyqqhyadack lrgdqplpiiatghlttvgasksdavrdiyigtldafpaqnfppadyialghihraqiiggmehvrycgspiplsfdecgkskyvhlvtf sngklesvenlnvpvtqpmavlkgdlasitaqleqwrdvsqeppvwldieittdeylhdiqrkiqalteslpvevllvrrsreqrervla sqqretlselsveevfnrrlaleeldesqqqrlqhlftttlhtlagehea. It should be understood that these amino acid sequences are exemplary and that one of skill in the art can identify SbcC and SbcD genes and proteins, including complexes, in other strains and cell lines based on homology.

In any of the foregoing embodiments, the sbcB gene can include a sequence having at least 95%, at least 98%, at least 99% or 100% sequence identity to SEQ ID NO: 11. In any of the foregoing embodiments, the recB gene can include a sequence having at least 95%, at least 98%, at least 99% or 100% sequence identity to SEQ ID NO: 12. In any of the foregoing embodiments, the recD gene can include a sequence having at least 95%, at least 98%, at least 99% or 100% sequence identity to SEQ ID NO: 13. In any of the foregoing embodiments, the recJ gene can include a sequence having at least 95%, at least 98%, at least 99% or 100% sequence identity to SEQ ID NO: 65.

In any of the foregoing embodiments, the uvrC gene can include a sequence having at least 95%, at least 98%, at least 99% or 100% sequence identity to SEQ ID NO: 14. In any of the foregoing embodiments, the mcrA gene can include a sequence having at least 95%, at least 98%, at least 99% or 100% sequence identity to SEQ ID NO: 15. In any of the foregoing embodiments, the mcrBC-hsd-mrr gene can include a sequence having at least 95%, at least 98%, at least 99% or 100% sequence identity to SEQ ID NO: 16-21.

In any of the foregoing embodiments, the engineered E. coli host cell can further include a vector. By way of example, but not limitation, the vector can be a non-viral transposon vector such as a transposase vector, a Sleeping Beauty transposon vector, a Sleeping Beauty transposase vector, a PiggyBac transposon vector, a PiggyBac transposase vector, an expression vector, and the like, a non-viral gene editing vector such as Homology-Directed Repair (HDR)/CRISPR-Cas9 vectors or a viral vector such as an AAV vector, an AAV rep cap vector, an AAV helper vector, an Ad helper vector, a Lentivirus vector, a Lentiviral envelope vector, a Lentiviral packaging vector, a Retroviral vector, a Retroviral envelope vector, a Retroviral packaging vector, a mRNA vector, or the like.

In any of the foregoing embodiments, where the E. coli host cell further includes a vector, the vector can include a nucleic acid sequence having a palindrome. A palindrome can be understood as a nucleic acid sequence in a double-stranded DNA molecule wherein reading in a certain direction on one strand matches the sequence reading in the opposite direction on the complementary strand, such that there are complementary portions along the one strand, where there is no intervening sequence between the complementary portions. By of example, but not limitation, the complementary sequences of the palindrome can each include about 10 to about 200 basepairs, about 15 and to about 200 basepairs, about 20 to about 200 basepairs, about 25 to about 200 basepairs, about 30 to about 200 basepairs, about 40 to about 200 basepairs, about 50 to about 200 basepairs, about 75 to about 200 basepairs, about 100 to about 200 base pairs, about 15 to about 200 basepairs, about 10 to about 150 basepairs, about 15 to about 150 basepairs, about 20 to about 150 base pairs, about 25 to about 150 basepairs, about 30 to about 150 basepairs, about 30 to about 150 basepairs, about 40 to about 150 basepairs, about 50 to about 150 basepairs, about 100 to about 150 base pairs, about 10 to about 140 basepairs, about 15 to about 140 basepairs, about 20 to about 140 basepairs, about 25 to about 140 basepairs, about 30 to about 140 basepairs, about 30 to about 140 basepairs, about 40 to about 140 basepairs, about 50 to about 140 basepairs, about 100 to about 140 basepairs, about 10 to about 100 basepairs, about 15 to about 100 basepairs, about 20 to about 100 basepairs, about 25 to about 100 base pairs, about 30 to about 100 basepairs, about 40 to about 100 basepairs, about 50 to about 100 basepairs, or about 10, 15, 20, 30, 40, 50, 60, 70, 80, 90, 100, 110, 120, 130, 140, 150, 160, 170, 180, 190, or 200 basepairs.

In any of the foregoing embodiments, where the E. coli host cell further includes a vector, the vector can include a nucleic acid sequence having at least one direct repeat. By way of example, but not limitation, the at least one direct repeat can include about 40 to 150 nucleotides, about 60 to about 120 nucleotides or about 90 nucleotides. By way of further example, but not limitation, the at least one direct repeat can be a simple repeat including a short sequence of DNA consisting of multiple repetitions of a single base, such as a polyA repeat, a polyT repeat, a polyC repeat or a polyG repeat, where the simple repeat includes about 40 to about 150 consecutive repeats of the same base, about 60 to about 120 consecutive repeats of the same base, or about 90 consecutive repeats of the same base. By way of further example, but not limitation, the polyA repeat can include 40 to 150 consecutive adenine nucleotides, 60 to 120 consecutive adenine nucleotides, or about 90 adenine nucleotides.

In any of the foregoing embodiment, where the E. coli host cell further includes a vector, the vector can include an inverted repeat sequence, a direct repeat sequence, a homopolymeric repeat sequence, an eukaryotic origin of replication, and a eukaryotic promoter enhancer sequence. By way of further example, the vector can include a sequence selected from the group consisting of a polyA repeat, a SV40 origin of replication, a viral LTR, a Lentiviral LTR, a Retroviral LTR, a transposon IR/DR repeat, a Sleeping Beauty transposon IR/DR repeat, an AAV ITR, a CMV enhancer, and a SV40 enhancer. By way of example, but not limitation, an AAV vector can contain an AAV ITR. In some embodiments, where the E. coli host cell further includes a vector, the vector can include a nucleic acid sequence having at least one inverted repeat sequence, which can also be an inverted terminal repeat such as, by way of example, but not limitation, an AAV ITR. Thus, in any of the foregoing embodiments, the vector can include an AAV ITR. It should be understood that an inverted repeat sequence is a single stranded sequence of nucleotides followed downstream by its reverse complement. It should be further understood that the single stranded sequence can be part of a double-stranded vector. The intervening sequence of nucleotides between the initial sequence and the reverse complement can be any length including zero. When the intervening length is zero, the composite sequence is a palindrome. When the intervening length is greater than zero, the composite sequence is an inverted repeat. In any of the foregoing embodiments, the intervening sequence can be 1 to about 2000 basepairs. By way of example, but not limitation, the inverted repeat, which can also be an inverted terminal repeat, can be separated by an intervening sequence comprising about 1 to about 2000 basepairs, about 5 to about 2000 basepairs, about 10 to about 2000 basepairs, about 25 to about 2000 basepairs, about 50 to about 2000 basepairs, about 100 to about 2000 basepairs, about 250 to about 2000 basepairs, about 500 to about 2000 basepairs, about 750 to about 2000 basepairs, about 1000 to about 2000 basepairs, about 1250 to about 2000 basepairs, about 1500 to about 2000 basepairs, about 1750 to about 2000 basepairs, about 1 to about 100 basepairs, about 1 to about 50 basepairs, about 1 to about 25 basepairs, about 1 to about 20 basepairs, about 1 to about 10 basepairs, about 1 to about 5 basepairs, or about 1, 2, 3, 4, 5, 6, 7, 8, 9, 10, 25, 50, 75, 100, 150, 200, 250, 300, 350, 400, 450, 500, 600, 700, 800, 900, 1000, 1100, 1200, 1300, 1400, 1500, 1600, 1700, 1800, 1900, or 2000 basepairs. By of example, but not limitation, the complementary portions of the inverted repeat can each include about 10 to about 200 basepairs, about 15 and to about 200 basepairs, about 20 to about 200 basepairs, about 25 to about 200 basepairs, about 30 to about 200 basepairs, about 40 to about 200 basepairs, about 50 to about 200 basepairs, about 75 to about 200 basepairs, about 100 to about 200 base pairs, about 15 to about 200 basepairs, about 10 to about 150 basepairs, about 15 to about 150 basepairs, about 20 to about 150 base pairs, about 25 to about 150 basepairs, about 30 to about 150 basepairs, about 30 to about 150 basepairs, about 40 to about 150 basepairs, about 50 to about 150 basepairs, about 100 to about 150 base pairs, about 10 to about 140 basepairs, about 15 to about 140 basepairs, about 20 to about 140 basepairs, about 25 to about 140 basepairs, about 30 to about 140 basepairs, about 30 to about 140 basepairs, about 40 to about 140 basepairs, about 50 to about 140 basepairs, about 100 to about 140 basepairs, about 10 to about 100 basepairs, about 15 to about 100 basepairs, about 20 to about 100 basepairs, about 25 to about 100 base pairs, about 30 to about 100 basepairs, about 40 to about 100 basepairs, about 50 to about 100 basepairs, or about 10, 15, 20, 30, 40, 50, 60, 70, 80, 90, 100, 110, 120, 130, 140, 150, 160, 170, 180, 190, or 200 basepairs. By way of example, but not limitation, the at least one inverted repeat can include an AAV ITR repeat that comprises sequences having at least 95%, at least 95%, at least 98%, at least 99% or 100% sequence identity to

(5’ AAV ITR, SEQ ID NO: 69)
ttggccactccctctctgcgcgctcgctcgctcactgaggccgggcgac
caaaggtcgcccgacgcccgggctttgcccgggcggcctcagtgagcga
gcgagcgcgcagagagggagtggccaactccatcactaggggttcct
and
(3’ AAV ITR, SEQ ID NO: 70))
aggaacccctagtgatggagttggccactccctctctgcgcgctcgctc
gctcactgaggccgggcgaccaaaggtcgcccgacgcccgggctttgcc
cgggcggcctcagtgagcgagcgagcgcgcagagagggagtggccaa.

Alternatively, in any of the foregoing embodiments, where the E. coli host cell further includes a vector, the vector can not include a nucleic acid sequence having a palindrome, direct repeat, or inverted repeat.

In any of the foregoing embodiments, the vector can be an AAV vector. In some embodiments, where the vector is an AAV vector, the AAV vector comprises an AAV ITR. In other embodiments, the vector can be a lentiviral vector, lentiviral envelope vector or lentiviral packaging vector. In still other embodiments, the vector can be a retroviral vector, retroviral envelope vector or a retroviral packaging vector. In yet other embodiments, the vector can be a transposase vector or a transposon vector. In still further embodiments, the vector can be a mRNA vector. By way of example, but not limitation, the mRNA vector can include a polyA repeat as described in the present disclosure.

In any of the foregoing embodiments, the vector can be a plasmid. In any of the foregoing embodiments, the vector can be a Rep protein dependent plasmid.

In any of the foregoing embodiments, the vector can further include a RNA selectable marker. By way of example, but not limitation, the RNA selectable marker can be a RNA-OUT. By way of further example, but not limitation, the RNA-OUT can have at least 95%, at least 98%, at least 99% or 100% sequence identity to a sequence selected from the group consisting of SEQ ID NO: 5 (gtagaattgg taaagagagt cgtgtaaaat atcgagttcg cacatcttgt tgtctgatta ttgatttttg gcgaaaccat ttgatcatat gacaagatgt gtatctacct taacttaatg attttgataa aaatcatta) and SEQ ID NO: 7 (gtagaattgg taaagagagt tgtgtaaaat attgagttcg cacatcttgt tgtctgatta ttgatttttg gcgaaaccat ttgatcatat gacaagatgt gtatctacct taacttaatg attttgataa aaatcatta) of WO 2019/183248 (SEQ ID NOs: 47 and 49, respectively). In some embodiments, the engineered E. coli host cell can include a corresponding RNA-IN sequence to permit regulation of a downstream marker by the RNA-OUT and that the RNA-OUT sequence corresponds to the RNA-IN.

In any of the foregoing embodiments, the vector can further include a RNA-OUT antisense repressor RNA. By way of example, but not limitation, the RNA-OUT antisense repressor RNA can have a sequence having at least 90%, at least 95%, at least 98%, at least 99% or 100% sequence identity to SEQ ID NO: 6 of WO 2019/183248 (SEQ ID NO: 48).

In any of the foregoing embodiments, the vector can further include a bacterial origin of replication. By way of example, but not limitation, the bacterial origin of replication can be selected from the group consisting of R6K, pUC and ColE2. By way of further example, but not limitation, the bacterial origin of replication can be a R6K gamma replication origin with at least 90%, at least 95%, at least 98%, at least 99% or 100% sequence identity to a sequence selected from the group consisting of SEQ ID NO: 1 (ggcttgttgt ccacaaccgt taaaccttaa aagctttaaa agccttatat attctttttt ttcttataaa acttaaaacc ttagaggcta tttaagttgc tgatttatat taattttatt gttcaaacat gagagcttag tacgtgaaac atgagagctt agtacgttag ccatgagagc ttagtacgtt agccatgagg gtttagttcg ttaaacatga gagcttagta cgttaaacat gagagcttag tacgtactat caacaggttg aactgctgat c), SEQ ID NO: 2 (ggcttgttgt ccacaaccat taaaccttaa aagctttaaa agccttatat attctttttt ttcttataaa acttaaaacc ttagaggcta tttaagttgc tgatttatat taattttatt gttcaaacat gagagcttag tacgtgaaac atgagagctt agtacattag ccatgagagc ttagtacatt agccatgagg gtttagttca ttaaacatga gagcttagta cattaaacat gagagcttag tacatactat caacaggttg aactgctgat c), SEQ ID NO: 3 (aaaccttaaa acctttaaaa gccttatata ttcttttttt tcttataaaa cttaaaacct tagaggctat ttaagttgct gatttatatt aattttattg ttcaaacatg agagcttagt acatgaaaca tgagagctta gtacattagc catgagagct tagtacatta gccatgaggg tttagttcat taaacatgag agcttagtac attaaacatg agagcttagt acatactatc aacaggttga actgctgatc), SEQ ID NO: 4 (tgtcagccgt taagtgttcc tgtgtcactg aaaattgctt tgagaggctc taagggcttc tcagtgcgtt acatccctgg cttgttgtcc acaaccgtta aaccttaaaa gctttaaaag ccttatatat tctttttttt cttataaaac ttaaaacctt agaggctatt taagttgctg atttatatta attttattgt tcaaacatga gagcttagta cgtgaaacat gagagcttag tacgttagcc atgagagctt agtacgttag ccatgagggt ttagttcgtt aaacatgaga gcttagtacg ttaaacatga gagcttagta cgtgaaacat gagagcttag tacgtactat caacaggttg aactgctgat cttcagatc) and SEQ ID NO: 18 (ggcttgttgt ccacaaccgt taaaccttaa aagctttaaa agccttatat attctttttt ttcttataaa acttaaaacc ttagaggcta tttaagttgc tgatttatat taattttatt gttcaaacat gagagcttag tacgtgaaac atgagagctt agtacgttag ccatgagagc ttagtacgtt agccatgagg gtttagttcg ttaaacatga gagcttagta cgttaaacat gagagcttag tacgttaaac atgagagctt agtacgtact atcaacaggt tgaactgctg atc) of WO 2019/183248 (SEQ ID NOs: 43-46 and 60, respectively), SEQ ID NO: 30 (ColE2 Origin (+7), 45 bp), SEQ ID NO: 31 (ColE2 Origin (+7, CpG free), 45 bp), SEQ ID NO: 32 (ColE2 Origin (Min), 38 bp), SEQ ID NO: 33 (ColE2 Origin (+16), 60 bp), and SEQ ID NO: 22 (pUC, 784 bp).

In any of the foregoing embodiments, the engineered E. coli host cell can further include a eukaryotic pUC-free minicircle expression vector that can include: (i) a eukaryotic region sequence encoding a gene of interest and having 5′ and 3′ ends; and (ii) a spacer region having a length of less than 1000, preferably less than 500, basepairs that links the 5′ and 3′ ends of the eukaryotic region sequence and that comprises a R6K bacterial replication origin and a RNA selectable marker. By way of example, but not limitation, the R6K bacterial replication origin and RNA selectable marker can have sequences as described in the present disclosure and as known in the art. Alternatively, in any of the foregoing embodiments, the engineered E. coli cell can further include a covalently closed circular plasmid having a backbone including a Pol III-dependent R6K origin of replication and an RNA-OUT selectable marker, where the backbone is less than 1000 bp, preferably less than 500 bp, and an insert including a structured DNA sequence. By way of example, but not limitation, the structured DNA sequence can include a sequence selected from the group consisting of an inverted repeat sequence, a direct repeat sequence, a homopolymeric repeat sequence, an eukaryotic origin of replication, and a euakaryotic promoter enhancer sequence. By way of further example, the structured DNA sequence can include a sequence selected from the group consisting of a polyA repeat, a SV40 origin of replication, a viral LTR, a Lentiviral LTR, a Retroviral LTR, a transposon IR/DR repeat, a Sleeping Beauty transposon IR/DR repeat, an AAV ITR, a CMV enhancer, and a SV40 enhancer. By way of example, but not limitation, the insert can be a transposase vector, an AAV vector, or a lentiviral vector. By way of example, but not limitation the Pol III-dependent R6K origin of replication can have a sequence having at least 90%, at least 95%, at least 98%, at least 99% or 100% sequence identity to a sequence selected from the group consisting of SEQ ID NO: 43, SEQ ID NO: 44, SEQ ID NO: 45, SEQ ID NO: 46, and SEQ ID NO: 60 (from SEQ ID Nos: 1-4 and 18 of WO2019/183248). By way of example, but not limitation, the RNA-OUT selectable marker can be an RNA-IN regulating RNA-OUT functional variant with at least 95%, at least 98%, at least 99% or 100% sequence identity to SEQ ID NO: 47 or SEQ ID NO: 49 (from SEQ ID Nos: 5 and 7 of WO 2019/183248). By way of further example, the RNA-OUT selectable marker can be a RNA-OUT antisense repressor RNA. By way of example, but not limitation, the RNA-OUT antisense repressor RNA can have a sequence having at least 90%, at least 95%, at least 98%, at least 99% or 100% sequence identity to SEQ ID NO: 6 of WO 2019/183248 (SEQ ID NO: 48).

It should be understood that a viability- or yield-reducing mutation refers to a mutation which reduces the viability or yield, respectively, of a cell line with respect to the cell line from which the mutated cell line is derived under the same culture conditions. It should be understood that such mutations can be engineered or naturally-occurring.

As disclosed herein, methods for the knockout or knockdown of a gene are well-known in the art, including, by way of example not limitation, the method disclosed in the Examples herein (recombineering), as well as P1 phage transduction, genome mass transfer, and CRISPR/Cas9. It should be understood that a gene knockout can result in either abolished expression of a protein or expression of a non-functional protein. Thus, the SbcCD complex may or may not be present in the bacterial host strains of the present disclosure, however, if present it is non-functional in the case of a knockout or has reduced activity as a nuclease in the case of a knockdown. It should be understood that embodiments of the disclosure can include a knockout or knockdown of SbcC, SbcD or both.

It is expected, without being bound to theory, that a knockout of SbcC or SbcD alone is sufficient to achieve the desired effect of the present invention because both proteins are essential subunits of the SbcCD nuclease (Connelly J C and Leach D R, Genes Cells 1:285, 1996). The sbcC and sbcD genes of E. coli encode a nuclease involved in palindrome inviability and genetic recombination. (Connelly J C and Leach D R, Genes Cells 1:285, 1996).

It should be understood that, within the present disclosure, an engineered E. coli host cell can include a vector as described herein. Vectors can include any suitable vector, including those described in those references incorporated herein by reference. For example, in some instances, the vectors can include a structured DNA sequence. In other instances, the vectors can not include a structured DNA sequence.

In some embodiments, the engineered E. coli host cell can further include a vector as understood in the present disclosure. Such vectors can be naturally-occurring or engineered. The vectors included in the engineered E. coli host cells of the present disclosure can include any of the features discussed herein and in the documents incorporated by reference. The vectors included in the engineered E. coli host cells of the present disclosure can, for example, include at least one inverted repeat, such as an inverted terminal repeat or palindrome, direct repeat or none of the foregoing structured DNA sequences.

Methods of Producing Engineered E. coli Host Cells

In some embodiments, a method for producing an engineered E. coli host cell is provided that includes the step of knocking out at least one gene selected from the group consisting of SbcC and SbcD in a starting E. coli cell that does not include an engineered viability- or yield-reducing mutation in any of sbcB, recB, recD, and recJ to yield the engineered E. coli host cell. In some embodiments, a method for producing an engineered E. coli host cell is provided that includes the step of knocking out at least one gene selected from the group consisting of SbcC and SbcD in a starting E. coli cell that does not include any engineered mutations in any of sbcB, recB, recD, and recJ to yield the engineered E. coli host cell. In some embodiments, a method for producing an engineered E. coli host cell is provided that includes the step of knocking out at least one gene selected from the group consisting of SbcC and SbcD in a starting E. coli cell that does not include any mutations in any of sbcB, recB, recD, and recJ to yield the engineered E. coli host cell.

In any of the foregoing embodiments, the starting E. coli cell can not include any engineered viability- or yield-reducing mutations in at least one of uvrC, mcrA, mcrBC-hsd-mir, and combinations thereof. In any of the foregoing embodiments, the starting E. coli cell can not include any mutations in at least one of uviC, mcrA, mcrBC-hsd-mrr, and combinations thereof. In any of the foregoing embodiments, the starting E. coli cell can not include any mutations in at least one of uviC, mcrA, mcrBC-hsd-mrr, and combinations thereof.

In any of the foregoing embodiments, the step of knocking out the at least one gene can not result in any mutation of sbcB, recB, recD and recJ. In any of the foregoing embodiments, the step of knocking out the at least one gene can not result in any mutations in at least one of uvrC, mcRA, mcrBC-hsd-mrr, and combinations thereof.

In any of the foregoing embodiments, the engineered E. coli host cell can not include an engineered viability- or yield reducing mutation in at least one of uviC, mcrA, mcrBC-hsd-mrr, and combinations thereof. In any of the foregoing embodiments, the engineered E. coli host cell can not include an engineered mutation in at least one of uviC, mcrA, mcrBC-hsd-mrr, and combinations thereof. In any of the foregoing embodiments, the engineered E. coli host cell can not include any mutation in at least one of uvrC, mcrA, mcrBC-hsd-mur, and combinations thereof.

In any of the foregoing embodiments, the engineered E. coli host cell can not include an engineered viability- or yield reducing mutation in sbcB, recB, recD and recJ. In any of the foregoing embodiments, the engineered E. coli host cell can not include an engineered mutation in sbcB, recB, recD and recJ. In any of the foregoing embodiments, the engineered E. coli host cell can not include any mutation in sbcB, recB, recD and recJ.

In any of the foregoing embodiments, the engineered E. coli host cell does not include a functional SbcCD complex. In any of the foregoing embodiments, the engineered E. coli host cell does not produce a SbcCD complex. Alternatively, in some embodiments, the engineered E. coli host cell produces a non-functional SbcCD complex.

It should be understood that in any of the foregoing method embodiments, the engineered E. coli host cell can be any E. coli host cell of the present disclosure.

In any of the foregoing embodiments, the SbcC gene can include a sequence having at least 90%, at least 95%, at least 98%, at least 99% or 100% sequence identity to SEQ ID NO: 9. In any of the foregoing embodiments, the SbcD gene can include a sequence having at least 90%, at least 95%, at least 98%, at least 99% or 100% sequence identity to SEQ ID NO: 10. It should be understood that this can apply to the gene prior to knockout or knockdown or after, i.e. in the engineered E. coli host cell.

In any of the foregoing embodiments, the sbcB gene can include a sequence having at least 95%, at least 98%, at least 99% or 100% sequence identity to SEQ ID NO: 11. In any of the foregoing embodiments, the recB gene can include a sequence having at least 95%, at least 98%, at least 99% or 100% sequence identity to SEQ ID NO: 12. In any of the foregoing embodiments, the recD gene can include a sequence having at least 95%, at least 98%, at least 99% or 100% sequence identity to SEQ ID NO: 13. In any of the foregoing embodiments, the recJ gene can include a sequence having at least 95%, at least 98%, at least 99% or 100% sequence identity to SEQ ID NO: 65.

In any of the foregoing embodiments, the uvrC gene can include a sequence having at least 95%, at least 98%, at least 99% or 100% sequence identity to SEQ ID NO: 14. In any of the foregoing embodiments, the mcrA gene can include a sequence having at least 95%, at least 98%, at least 99% or 100% sequence identity to SEQ ID NO: 15. In any of the foregoing embodiments, the mcrBC-hsd-mrr gene can include a sequence having at least 95%, at least 98%, at least 99% or 100% sequence identity to SEQ ID NOs: 16-21.

Methods for Vector Production

In some embodiments, a method for improved vector production is provided that includes the step of transfecting an engineered E. coli host cell with a vector yield a transfected host cell and incubating the transfected host cell under conditions sufficient to replicate the vector, where the E. coli host cell does not include an engineered viability- or yield-reducing mutation in any of sbcB, recB, recD, and recJ. It should be understood that the vector used to transfect the engineered E. coli host cell can be any vector as described in the present disclosure, including the embodiments disclosed where an engineered E. coli host cell of the present disclosure includes a vector.

In some embodiments, a method for improved vector production is provided that includes the step of incubating a transfected host cell that is an engineered E. coli host cell that includes a vector and that does not include an engineered viability- or yield-reducing mutation in any of sbcB, recB, recD, and recJ, that includes a vector, and incubating the transfected host cell under conditions sufficient to replicate the vector.

In any of the foregoing embodiments, it should be understood that the engineered E. coli host cell can be any engineered E. coli host cell of the present disclosure.

In any of the foregoing embodiments, the methods can further include isolating the vector from the transfected host cell.

In any of the foregoing embodiments, the step of incubating the transfected host cell, whether transfected or after transfection with a vector, can be performed by a fed-batch fermentation, where the fed-batch fermentation comprises growing the engineered E. coli host cells at a reduced temperature during a first portion of the fed-batch phase, which can be under growth-restrictive conditions, followed by a temperature up-shift to a higher temperature during a second portion of the fed-batch phase. By way of example, the reduced temperature can be about 28-30° C. and the higher temperature can be about 37-42° C. By way of example, the first portion can be about 12 hours and the second portion can be about 8 hours. It should be understood that where the fed-batch fermentation with a temperature upshift is used, the engineered E. coli host cell can have a lambda repressor and Rep protein that is under the control of a P_{L} promoter that can be regulated by the lambda repressor, which can be temperature-sensitive.

In any of the foregoing embodiments, the plasmid yield after incubating the transfected host cell under conditions sufficient to replicate the vector can be higher than for the cell line from which the engineered E. coli host cell was derived treated under the same conditions. In any of the foregoing embodiments, the plasmid yield after incubating the transfected host cell under conditions sufficient to replicate the vector can be higher than for SURE2, SURE, Stbl2, Stbl3, or Stbl4 cells treated under the same conditions.

In any of the foregoing embodiments, the SbcC gene can include a sequence having at least 90%, at least 95%, at least 98%, at least 99% or 100% sequence identity to SEQ ID NO: 9. In any of the foregoing embodiments, the SbcD gene can include a sequence having at least 90%, at least 95%, at least 98%, at least 99% or 100% sequence identity to SEQ ID NO: 10. It should be understood that this can apply to the gene prior to knockout or knockdown or after, i.e. in the engineered E. coli host cell.

In any of the foregoing embodiments, the sbcB gene can include a sequence having at least 95%, at least 98%, at least 99% or 100% sequence identity to SEQ ID NO: 11. In any of the foregoing embodiments, the recB gene can include a sequence having at least 95%, at least 98%, at least 99% or 100% sequence identity to SEQ ID NO: 12. In any of the foregoing embodiments, the recD gene can include a sequence having at least 95%, at least 98%, at least 99% or 100% sequence identity to SEQ ID NO: 13. In any of the foregoing embodiments, the recJ gene can include a sequence having at least 95%, at least 98%, at least 99% or 100% sequence identity to SEQ ID NO: 65.

In any of the foregoing embodiments, the uvrC gene can include a sequence having at least 95%, at least 98%, at least 99% or 100% sequence identity to SEQ ID NO: 14. In any of the foregoing embodiments, the mcrA gene can include a sequence having at least 95%, at least 98%, at least 99% or 100% sequence identity to SEQ ID NO: 15. In any of the foregoing embodiments, the mcrBC-hsd-mrr gene can include a sequence having at least 95%, at least 98%, at least 99% or 100% sequence identity to SEQ ID NOs: 16-21.

It should be understood that in any of the foregoing embodiments, the vector that is transfected into the engineered E. coli host cell can be any vector as described herein.

It should be understood that in any of the foregoing embodiments, the engineered E. coli host cell can include a knockdown of SbcC, SbcD, or both, rather than a knockout. The knockdown can result in reduced expression and/or reduced activity of the SbcCD complex.

The reduction can be by at least 10%, at least 20%, at least 30%, at least 40%, at least 50%, at least 60%, at least 70%, at least 80%, at least 90%, at least 95%, at least 98%, at least 99% or more.

The bacterial host strains and methods of the present disclosure will now be described with reference to the following non-limiting examples.

### EXAMPLES

The majority of therapeutic plasmids use the pUC origin which is a high copy derivative of the pMB1 origin (closely related to the ColE1 origin). For pMB1 replication, plasmid DNA synthesis is unidirectional and does not require a plasmid borne initiator protein. The pUC origin is a copy up derivative of the pMB1 origin that deletes the accessory ROP (rom) protein and has an additional temperature sensitive mutation that destabilizes the RNAI/RNAII interaction. Shifting of a culture containing these origins from 30 to 42° C. leads to an increase in plasmid copy number. pUC plasmids can be produced in a multitude of E. coli cell lines.

In the following examples, for shake flask production proprietary Plasmid+ shake culture medium was used. The seed cultures were started from glycerol stocks or colonies and streaked onto LB medium agar plates containing 50 pg/mL antibiotic (for ampR or kanR selection plasmids) or 6% sucrose (for RNA-OUT selection plasmids). The plates were grown at 30-32° C.; cells were resuspended in media and used to provide approximately 2.5 OD₆₀₀ inoculums for the 500 mL Plasmid+ shake flasks that contained 50 pg/mL antibiotic for ampR or kanR selection plasmids or 0.5% sucrose to select for RNA-OUT plasmids. Flask were grown with shaking to saturation at the growth temperatures as indicated.

In the following examples, HyperGRO fermentations were performed using proprietary fed-batch media (NTC3019, HyperGRO media) in New Brunswick BioFlo 110 bioreactors as described (U.S. Pat. No. 7,943,377, which is incorporated herein by reference in its entirety). The seed cultures were started from glycerol stocks or colonies and streaked onto LB medium agar plates containing 50 pg/mL antibiotic (for ampR or kanR selection plasmids) or 6% sucrose (for RNA-OUT selection plasmids). The plates were grown at 30-32° C.; cells were resuspended in media and used to provide approximately 0.1% inoculums for the fermentations that contained 50 pg/mL antibiotic for ampR or kanR selection plasmids or 0.5% sucrose for RNA-OUT plasmids. HyperGRO temperature shifts were as indicated.

In the following examples, culture samples were taken at key points and regular intervals during all fermentations. Samples were analyzed immediately for biomass (OD₆₀₀) and for plasmid yield. Where plasmid yield was determined, the analysis was performed by quantification of plasmid obtained from Qiagen Spin Miniprep Kit preparations as described in U.S. Pat. No. 7,943,377. Briefly, cells were alkaline lysed, clarified, plasmid was column purified, and eluted prior to quantification. Plasmid quality was determined by agarose gel electrophoresis analysis (AGE) and was performed on 0.8-1% Tris/acetate/EDTA (TAE) gels as described in U.S. Pat. No. 7,943,377.

Strains used in the following examples included:

RNA-OUT antibiotic free selectable marker background: Antibiotic-free selection is performed in E. coli strains containing phage lambda attachment site chromosomally integrated pCAH63-CAT RNA-IN-SacB (P5/6 6/6) for example NTC4862 as described in WO 2008/153733. SacB (Bacillus subtilis levansucrase) is a counterselectable marker which is lethal to E. coli cells in the presence of sucrose. Translation of SacB from the RNA-IN-SacB transcript is inhibited by plasmid encoded RNA-OUT. This facilitates plasmid selection in the presence of sucrose, by inhibition of SacB mediated lethality.

R6K origin vector replication background: The R6K gamma plasmid replication origin requires a single plasmid replication protein n that binds as a replication initiating monomer to multiple repeated ‘iteron’ sites (seven core repeats containing TGAGNG consensus) and as a replication inhibiting dimer to repressive sites (TGAGNG) and to iterons with reduced affinity. Replication requires multiple host factors including IHF, DnaA, and primosomal assembly proteins DnaB, DnaC, DnaG (Abhyankar et al., 2003 J Biol Chem 278:45476-45484). The R6K core origin contains binding sites for DnaA and IHF that affect plasmid replication since n, IHF and DnaA interact to initiate replication.

Different versions of the R6K gamma replication origin have been utilized in various eukaryotic expression vectors, for example pCOR vectors (Soubrier et al., 1999, Gene Therapy 6:1482-88) and a CpG free version in pCpGfree vectors (Invivogen, San Diego Calif.), and pGM169 (University of Oxford). A highly minimalized 6 iteron R6K gamma derived replication origin that contains core sequences required for replication (including the DnaA box and stb 1-3 sites; Wu et al., 1995. J Bacteriol. 177: 6338-6345), but with the upstream n dimer repressor binding sites and downstream n promoter deleted (by removing one copy of the iterons) was described in WO 2014/035457 and included herein by reference (SEQ ID NO: 1 from WO 2019/183248 (SEQ ID NO: 43)). This R6K origin contains 6 tandem direct repeat iterons. The NTC9385R Nanoplasmid™ vector including this minimalized R6K origin and the RNA-OUT AF (antibiotic-free) selectable marker in the spacer region, was described in WO 2014/035457 and included herein by reference. An R6K origin containing 7 tandem direct repeat iterons and an R6K origin contains 6 tandem direct repeat iterons and a single CpG residue were described in WO 2019183248 and included herein by reference. Use of a conditional replication origin such as R6K gamma that requires a specialized cell line for propagation adds a safety margin since the vector will not replicate if transferred to a patient's endogenous flora.

Typical R6K production strains express from the genome the π protein derivative PIR116 that contains a P106L substitution that increases copy number (by reducing π dimerization; π monomers activate while π dimers repress). Fermentation results with pCOR (Soubrier et al., Supra, 1999) and pCpG plasmids (Hebel H L, Cai Y, Davies L A, Hyde S C, Pringle I A, Gill D R. 2008. Mol Ther 16: S110) were low, around 100 mg/L in PIR116 cell lines.

Mutagenesis of the pir-116 replication protein and selection for increased copy number has been used to make new production strains. For example, the TEX2pir42 strain contains a combination of P106L and P42L. The P42L mutation interferes with DNA looping replication repression. The TEX2pir42 cell line improved copy number and fermentation yield with pCOR plasmids with reported yields of 205 mg/L (Soubrier F. 2004. International Patent Application WO2004/033664).

Other combinations of n copy number mutants that improve copy number include ‘P42L and P113S’ and ‘P42L, P106L and F107S’ (Abhyankar et al., 2004. J Biol Chem 279:6711-6719).

WO 2014/035457 describes host strains expressing phage HK022 attachment site integrated pL promoter heat inducible π P42L, P106L and F107S high copy mutant replication (Rep) protein for selection and propagation of R6K origin Nanoplasmid™ vectors.

RNA-OUT selectable marker-R6K plasmid propagation and fermentations described in WO 2014/035457 were performed using heat inducible ‘P42L, P106L and F107S’ π copy number mutant cell lines such as DH5α host strain NTC711772=DH5α dcm−att_{λ}::P_{c}-RNA-IN-SacB, catR; att_{HK022}::pL (OL1-G to T) P42L-P106L-F107S (P3−), SpecR StrepR. Production yields up to 695 mg/L were reported.

Additional R6K origin ‘copy cutter’ host cell lines were created and disclosed in Williams 2019 VIRAL AND NON-VIRAL NANOPLASMID VECTORS WITH IMPROVED PRODUCTION World Patent Application WO2019/183248 including:
NTC1050811 DH5α att_{λ}::P_{c}-RNA-IN-SacB, catR; att_{HK022}::pL (OL1-G to T) P42L-P106I-F107S P113S (P3−), SpecR StrepR; att_{φ80}::pARA-CI857ts, tetR=pARA-CI857ts derivative of NTC940211. This ‘copy cutter’ host strain contains a phage (980 attachment site chromosomally integrated copy of a arabinose inducible CI857ts gene. Addition of arabinose to plates or media (e.g. to 0.2-0.4% final concentration) induces pARA mediated CI857ts repressor expression which reduces copy number at 30° C. through CI857ts mediated downregulation of the Rep protein expressing pL promoter [i.e. additional CI857ts mediates more effective downregulation of the pL (OL1-G to T) promoter at 30° C.]. Copy number induction after temperature shift to 37-42° C. is not impaired since the CI857ts repressor is inactivated at these elevated temperatures. A dcm−derivative (NTC1050811 dcm−) is used in cases where dcm methylation is undesirable. NTC1050811-HF is a derivative of the NTC1050811 cell line that includes a second copy of the RNA-IN-SacB expression cassette, and that does not have mutations in sbcB, recB, recD, recJ, uvrC, mcrA or mcrBC-hsd-mrr.

In each case, both strains (NTC1050811 and NTC1050811-HF) contain a phage (980 attachment site chromosomally integrated copy of a arabinose inducible CI857ts gene. Addition of arabinose to plates or media (e.g. to 0.2-0.4% final concentration) induces pARA mediated CI857ts repressor expression which reduces copy number at 30° C. through CI857ts mediated downregulation of the Rep protein expressing pL promoter [i.e. additional CI857ts mediates more effective downregulation of the pL (OL1-G to T) promoter at 30° C.]. Copy number induction after temperature shift to 37-42° C. is not impaired since the CI857ts repressor is inactivated at these elevated temperatures. These ‘copy cutter host strains’ increase the R6K vector temperature upshift copy number induction ratio by reducing the copy number at 30° C. This is advantageous for production of large, toxic, or dimerization prone R6K origin vectors.

Nanoplasmid™ production yields are improved with the quadruple mutant heat inducible pL (OL1-G to T) P42L-P106I-F107S P113S (P3−) described in WO 2019/183248 compared to the triple mutant heat inducible pL (OL1-G to T) P42L-P106L-F107S (P3−) described in WO 2014/035457. Yields in excess of 2 g/L Nanoplasmid™ have been obtained with the quadruple mutant NTC1050811 cell line (WO 2019/183248).

Use of a conditional replication origin such as these R6K origins that requires a specialized cell line for propagation adds a safety margin since the vector will not replicate if transferred to a patient's endogenous flora.

RNA-OUT production hosts described in WO 2019/183248 were modified to create HF hosts. SacB (Bacillus subtilis levansucrase) is a counterselectable marker which is lethal to E. coli cells in the presence of sucrose. Translation of SacB from the RNA-IN-SacB transcript is inhibited by plasmid encoded RNA-OUT. This facilitates plasmid selection in the presence of sucrose, by inhibition of SacB mediated lethality. Mutation of the chromosomal copy of the RNA-IN-SacB expression cassette that eliminate SacB expression are sucrose resistant (in the absence of plasmid). The presence of the second copy of the RNA-IN-SacB expression cassette dramatically reduces the numbers of sucrose resistant (in the absence of plasmid) colonies, since each individual RNA-IN-SacB expression cassette copy mediates sucrose lethality in the absence of plasmid very rare mutations to both chromosomal copies of RNA-IN-SacB expression cassettes is necessary to obtain sucrose resistant in the absence of plasmid.

NTC1011592 Stbl4 attλ::P_{c}-RNA-IN-SacB, catR (WO 2019/183248) was also used.

In the following examples, production strains that were not altered included: DH5α, Sure2, Stbl2, Stbl3 or Stbl4.

### Example 1: Preparation of SbcCD Knockout Strains

SbcCD knockout strains were produced using Red Gam recombination cloning as described in Datsenko and Wanner, PNAS USA 97:6640-6645 (2000). The pKD4 plasmid (Datsenko and Wanner, 2000) was PCR amplified with the following primers to introduce SbcC and SbcD targeting homology arms.

SEQ ID NO 1 (SbccR-pKD4):
CCCTCTGTATTCATTATCCTGCTGAATAGTTATTTCACTGCAAACGTAC
TCATATGAATATCCTCCTTAG
SEQ ID NO 2 (SbcdF-pKD4):
TCTGTTTGGGTATAATCGCGCCCATGCTTTTTCGCCAGGGAACCGTTAT
GTGTAGGCTGGAGCTGCTTCG

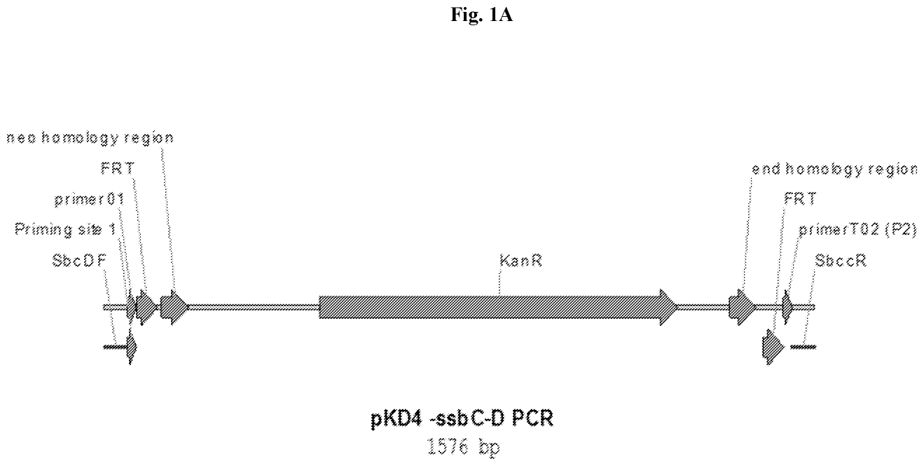

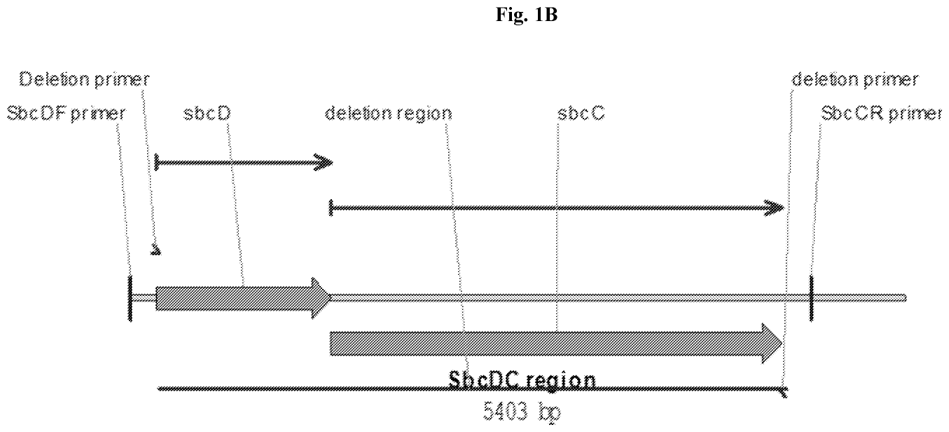

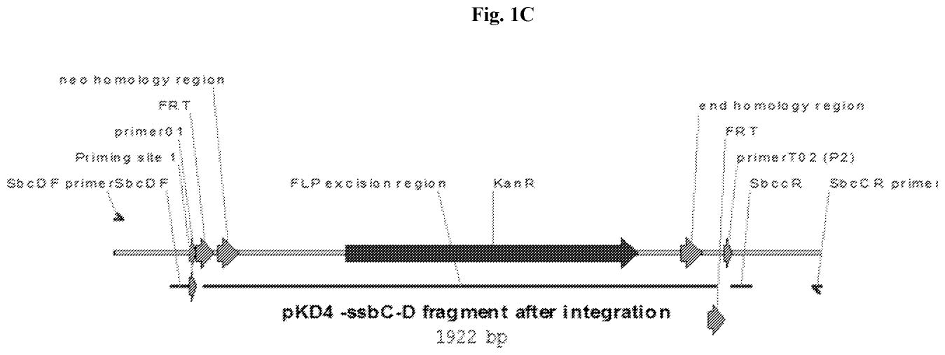

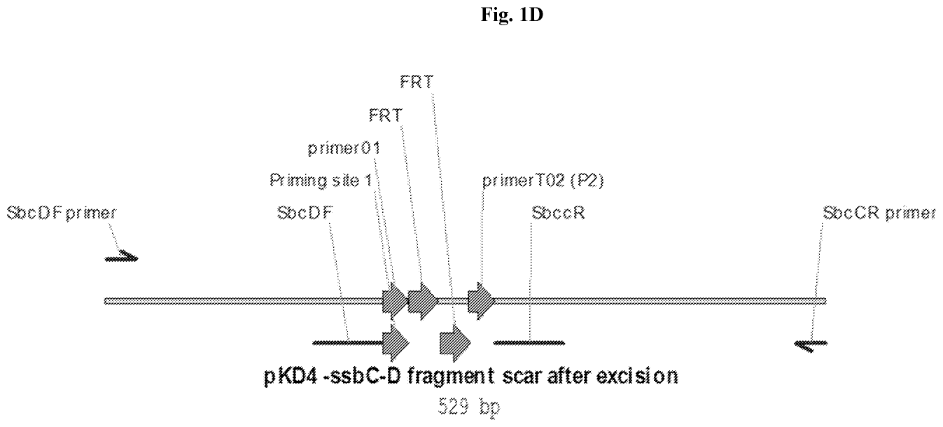

The 1.6 kb PCR product (SEQ ID NO: 5, tctgtttgggtataatcgcgcccatgctttttcgccagggaaccgttatgtgtaggctggagctgcttcgaagttcctatactttctagagaata ggaacttcggaataggaacttcaagatcccctcacgctgccgcaagcactcagggcgcaagggctgctaaaggaagcggaacacgta gaaagccagtccgcagaaacggtgctgaccccggatgaatgtcagctactgggctatctggacaagggaaaacgcaagcgcaaaga gaaagcaggtagcttgcagtgggcttacatggcgatagctagactgggcggttttatggacagcaagcgaaccggaattgccagctgg ggcgccctctggtaaggttgggaagccctgcaaagtaaactggatggctttcttgccgccaaggatctgatggcgcaggggatcaagat ctgatcaagagacaggatgaggatcgtttcgcatgattgaacaagatggattgcacgcaggttctccggccgcttgggtggagaggctat tcggctatgactgggcacaacagacaatcggctgctctgatgccgccgtgttccggctgtcagcgcaggggcgcccggttctttttgtca agaccgacctgtccggtgccctgaatgaactgcaggacgaggcagcgcggctatcgtggctggccacgacgggcgttccttgcgcag ctgtgctcgacgttgtcactgaagcgggaagggactggctgctattgggcgaagtgccggggcaggatctcctgtcatctcaccttgctc ctgccgagaaagtatccatcatggctgatgcaatgcggcggctgcatacgcttgatccggctacctgcccattcgaccaccaagcgaaa catcgcatcgagcgagcacgtactcggatggaagccggtcttgtcgatcaggatgatctggacgaagagcatcaggggctcgcgcca gccgaactgttcgccaggctcaaggcgcgcatgcccgacggcgaggatctcgtcgtgacccatggcgatgcctgcttgccgaatatcat ggtggaaaatggccgcttttctggattcatcgactgtggccggctgggtgtggcggaccgctatcaggacatagcgttggctacccgtga tattgctgaagagcttggcggcgaatgggctgaccgcttcctcgtgctttacggtatcgccgctcccgattcgcagcgcatcgccttctatc gccttcttgacgagttcttctgagcgggactctggggttcgaaatgaccgaccaagcgacgcccaacctgccatcacgagatttcgattcc accgccgccttctatgaaaggttgggcttcggaatcgttttccgggacgccggctggatgatcctccagcgcggggatctcatgctggag ttcttcgcccaccccagcttcaaaagcgctctgaagttcctatactttctagagaataggaacttcggaataggaactaaggaggatattcat atgagtacgtttgcagtgaaataactattcagcaggataatgaatacagaggg) (FIG. 1A) was purified and DpnI digested (to eliminate template plasmid). The host strain in which the SbcCD genes were to be knocked out was transformed with pKD46-RecApa recombineering plasmid (WO 2008/153731, which is incorporated by reference herein in its entirety) and transformants selected for ampicillin resistance. Electrocompetent cells of the transformed cell line were made by growth in LB medium including 50 pg/mL ampicillin, at approximately 0.05 OD₆₀₀, arabinose was added to 0.2% to induce recombineering gene expression, the cells were grown to mid-log phase and electrocompetent cells made by centrifugation and resuspension in 10% glycerol at 1/200 original volume. 5 pL of DpnI-digested, purified PCR product was electroporated into 25 pL electrocompetent cells after which 1 mL of SOC medium was added. The cells were outgrown for 2 hours at 30° C., plated on LB agar plates containing 20 pg kanamycin and grown at 37° C. overnight. Individual kanR colonies were screened for ΔSbcDC::kanR by using SbcDF and SbcCR primers as described below.

SEQ ID NO 3 (SbcDF primer):
cgtctcgccatgatttgccctg
SEQ ID NO 4 (SbcCR primer):
cgttatgcgccagctccgtgag
Host: Product of SbcDF and SbcCR primers = 4.8 kb (FIG. 1B),
(SEQ ID NO: 6
cgtctcgccatgatttgccctgttgtaataaataggttgcgatcattaatgcgacgtcattatgcgtcagatttatgacagatttat
gaaaagctcgtcgcacatatcttcaggttattgatttccgtggcgcagaaaaaagcaaatggcacatctgtttgggtataatc
gcgcccatgctttttcgccagggaaccgttatgcgcatccttcacacctcagactggcatctcggccagaacttctacagtaa
aagccgcgaagctgaacatcaggcttttcttgactggctgctggagacagcacaaacccatcaggtggatgcgattattgtt
gccggtgatgttttcgataccggctcgccgcccagttacgcccgcacgttatacaaccgttttgttgtcaatttacagcaaact
ggctgtcatctggtggtactggcaggaaaccatgactcggtcgccacgctgaatgaatcgcgcgatatcatggcgttcctc
aatactaccgtggtcgccagcgccggacatgcgccgcaaatcttgcctcgtcgcgacgggacgccaggcgcagtgctgt
gccccattccgtttttacgtccgcgtgacattattaccagccaggcggggcttaacggtattgaaaaacagcagcatttactg
gcagcgattaccgattattaccaacaacactatgccgatgcctgcaaactgcgcggcgatcagcctctgcccatcatcgcc
acgggacatttaacgaccgtgggggccagtaaaagtgacgccgtgcgtgacatttatattggcacgctggacgcgtttccg
gcacaaaactttccaccagccgactacatcgcgctcgggcatattcaccgcgcacagattattggcggcatggaacatgtt
cgctattgcggctcccccattccactgagttttgatgaatgcggtaagagtaaatatgtccatctggtgacattttcaaacggc
aaattagagagcgtggaaaacctgaacgtaccggtaacgcaacccatggcagtgctgaaaggcgatctggcgtcgattac
cgcacagctggaacagtggcgcgatgtatcgcaggagccacctgtctggctggatatcgaaatcactactgatgagtatct
gcatgatattcagcgcaaaatccaggcattaaccgaatcattgcctgtcgaagtattgctggtacgtcggagtcgtgaacag
cgcgagcgtgtgttagccagccaacagcgtgaaaccctcagcgaactcagcgtcgaagaggtgttcaatcgccgtctgg
cactggaagaactggatgaatcgcagcagcaacgtctgcagcatcttttcaccacgacgttgcataccctcgccggagaa
cacgaagcatgaaaattctcagcctgcgcctgaaaaacctgaactcattaaaaggcgaatggaagattgatttcacccgcg
agccgttcgccagcaacgggctgtttgctattaccggcccaacaggtgcggggaaaaccaccctgctggacgccatttgt
ctggcgctgtatcacgaaactccgcgtctctctaacgtttcacaatcgcaaaatgatctcatgacccgcgataccgccgaat
gtctggcggaggtggagtttgaagtgaaaggtgaagcgtaccgtgcattctggagccagaatcgggcgcgtaaccaacc
cgacggtaatttgcaggtgccacgcgtagagctggcgcgctgcgccgacggcaaaattctcgccgacaaagtgaaagat
aagctggaactgacagcgacgttaaccgggctggattacgggcgcttcacccgttcgatgctgctttcgcaggggcaattt
gctgccttcctgaatgccaaacccaaagaacgcgcggaattgctcgaggagttaaccggcactgaaatctacgggcaaat
ctcggcgatggtttttgagcagcacaaatcggcccgcacagagctggagaagctgcaagcgcaggccagcggcgtcac
gttgctcacgccggaacaagtgcaatcgctgacagcgagtttgcaggtacttactgacgaagaaaaacagttaattaccgc
gcagcagcaagaacaacaatcgctaaactggttaacgcgtcaggacgaattgcagcaagaagccagccgccgtcagca
ggccttgcaacaggcgttagccgaagaagaaaaagcgcaacctcaactggcggcgcttagtctggcacaaccggcacg
aaatcttcgtccacactgggaacgcatcgcagaacacagcgcggcgctggcgcatattcgccagcagattgaagaagta
aatactcgcttacagagcacaatggcgcttcgcgcgagcattcgccaccacgcggcgaagcagtcagcagaattacagc
agcagcaacaaagcctgaatacctggttacaggaacacgaccgcttccgtcagtggaacaacgaaccggcgggttggc
gtgcgcagttctcccaacaaaccagcgatcgcgagcatctgcggcaatggcagcaacagttaacccatgctgagcaaaa
acttaatgcgcttgcggcgatcacgttgacgttaaccgccgatgaagttgctaccgccctggcgcaacatgctgagcaacg
cccactgcgtcagcacctggtcgcgctgcatggacagattgttccccaacaaaaacgtctggcgcagttacaggtcgctat
ccagaatgtcacgcaagaacagacgcaacgtaacgccgcacttaacgaaatgcgccagcgttataaagaaaagacgca
gcaacttgccgatgtgaaaaccatttgcgagcaggaagcgcgcatcaaaacgctggaagctcaacgtgcacagttacag
gcgggtcagccttgcccactttgtggttccaccagccacccggcggtcgaggcgtatcaggcgctggagcctggcgttaa
tcagtctcgattactggcgctggaaaacgaagttaaaaagctcggtgaagaaggtgcgacgctacgtgggcaactggacg
ccataacaaagcagcttcagcgtgatgaaaacgaagcgcaaagcctccgacaagatgagcaagcacttactcaacaatg
gcaagccgtcacggccagcctcaatatcaccttgcagccactggacgatattcaaccgtggctggatgcacaagatgagc
acgaacgccagctgcggttactcagccaacggcatgaattacaagggcagattgccgcgcataatcagcaaattatccag
tatcaacagcaaattgaacaacgccagcaactacttttaacgacattgacgggttatgcactgacattgccacaggaagatg
aagaagagagctggttggcgacacgtcagcaagaagcgcagagctggcagcaacgccagaacgaattaaccgcgctg
caaaaccgtattcagcagctgacgccgattctggaaacgttgccgcaaagtgatgaactcccgcactgcgaagaaactgt
ggtattggaaaactggcggcaggtacatgaacaatgtctcgcattacacagccagcagcagacgttacagcaacaggatg
ttctggcggcgcaaagtctgcaaaaagcccaggcgcagtttgacaccgcgctacaggccagcgtctttgacgatcagcag
gcgttccttgcggcgctaatggatgaacaaacactaacgcagctggaacagctcaagcagaatctggaaaaccagcgcc
gtcaggcgcaaactctggtcactcagacagcagaaacgctggcacagcatcaacaacaccgacctgacgacgggttgg
ctctcactgtgacggtggagcagattcagcaagagttagcgcaaactcaccaaaagttgcgtgaaaacaccacgagtcaa
ggcgagattcgccagcagctgaagcaggatgcagataaccgtcagcaacaacaaaccttaatgcagcaaattgctcaaat
gacgcagcaggttgaggactggggatatctgaattcgctaataggttccaaagagggcgataaattccgcaagtttgccca
ggggctgacgctggataatttagtccatctcgctaatcagcaacttacccggctgcacgggcgctatctgttacagcgcaaa
gccagcgaggcgctggaagtcgaggttgttgatacctggcaggcagatgcggtacgcgatacccgtaccctttccggcg
gcgaaagtttcctcgttagtctggcgctggcgctggcgctttcggatctggtcagccataaaacacgtattgactcgctgttc
cttgatgaaggttttggcacgctggatagcgaaacgctggataccgcccttgatgcgctggatgccctgaacgccagtggc
aaaaccatcggtgtgattagccacgtagaagcgatgaaagagcgtattccggtgcagatcaaagtgaaaaagatcaacgg
cctgggctacagcaaactggaaagtacgtttgcagtgaaataactattcagcaggataatgaatacagaggggcgaattat
ctcttggccttgctggtcgttatcctgcaagctatcactttattggctacggtgattggtagccgttctggtggttgtgatggtgg
tatgaaaaaagtcattttatctttggctctgggcacgtttggtttggggatggccgaatttggcattatgggcgtgctcacgga
gctggcgcataacgtaggaatttcgattcctgccgccgggcatatgatctcgtattatgcactgggggtggtggtcggtgcg
ccaatcatcgcactcttttccagccgctactcactcaaacatatcttgttgtttctggtggcgttgtgcgtcattggcaacgccat
gttcacgctctcttcgtcttacctgatgctcgccattggtcggctggtatccggctttccgcatggcgcattttttggcgtcgga
gcgatcgtgttatcaaaaattatcaaacccggaaaagtcaccgccgccgtggcggggatggtttccgggatgacagtcgc
caatttgctgggcattccgctgggaacgtatttaagtcaggaatttagctggcgttacacctttttattgatcgctgtttttaatatt
gcggtgatggcatcggtctatttttgggtgccagatattcgcgacgaggcgaaaggaaatctgcgcgaacaatttcacttttt
gcgcagcccggccccgtggttaattttcgccgccacgatgtttggcaacgcaggtgtgtttgcctggttcagctacgtaaag
ccatacatgatgtttatttccggtttttcggaaacggcgatgacctttattatgatgttagtt)
Host ASbcDC::kanR: Product of SbcDF and SbcCR primers = 1.9 kb (FIG. 1C),
(SEQ ID NO: 7
cgtctcgccatgatttgccctgttgtaataaataggttgcgatcattaatgcgacgtcattatgcgtcagatttatgacagatttat
gaaaagctcgtcgcacatatcttcaggttattgatttccgtggcgcagaaaaaagcaaatggcacatctgtttgggtataatc
gcgcccatgctttttcgccagggaaccgttatgtgtaggctggagctgcttcgaagttcctatactttctagagaataggaact
tcggaataggaacttcaagatcccctcacgctgccgcaagcactcagggcgcaagggctgctaaaggaagcggaacac
gtagaaagccagtccgcagaaacggtgctgaccccggatgaatgtcagctactgggctatctggacaagggaaaacgca
agcgcaaagagaaagcaggtagcttgcagtgggcttacatggcgatagctagactgggcggttttatggacagcaagcg
aaccggaattgccagctggggcgccctctggtaaggttgggaagccctgcaaagtaaactggatggctttcttgccgcca
aggatctgatggcgcaggggatcaagatctgatcaagagacaggatgaggatcgtttcgcatgattgaacaagatggattg
cacgcaggttctccggccgcttgggtggagaggctattcggctatgactgggcacaacagacaatcggctgctctgatgc
cgccgtgttccggctgtcagcgcaggggcgcccggttctttttgtcaagaccgacctgtccggtgccctgaatgaactgca
ggacgaggcagcgcggctatcgtggctggccacgacgggcgttccttgcgcagctgtgctcgacgttgtcactgaagcg
ggaagggactggctgctattgggcgaagtgccggggcaggatctcctgtcatctcaccttgctcctgccgagaaagtatcc
atcatggctgatgcaatgcggcggctgcatacgcttgatccggctacctgcccattcgaccaccaagcgaaacatcgcatc
gagcgagcacgtactcggatggaagccggtcttgtcgatcaggatgatctggacgaagagcatcaggggctcgcgcca
gccgaactgttcgccaggctcaaggcgcgcatgcccgacggcgaggatctcgtcgtgacccatggcgatgcctgcttgc
cgaatatcatggtggaaaatggccgcttttctggattcatcgactgtggccggctgggtgtggcggaccgctatcaggacat
agcgttggctacccgtgatattgctgaagagcttggcggcgaatgggctgaccgcttcctcgtgctttacggtatcgccgct
cccgattcgcagcgcatcgccttctatcgccttcttgacgagttcttctgagcgggactctggggttcgaaatgaccgacca
agcgacgcccaacctgccatcacgagatttcgattccaccgccgccttctatgaaaggttgggcttcggaatcgttttccgg
gacgccggctggatgatcctccagcgcggggatctcatgctggagttcttcgcccaccccagcttcaaaagcgctctgaa
gttcctatactttctagagaataggaacttcggaataggaactaaggaggatattcatatgagtacgtttgcagtgaaataact
attcagcaggataatgaatacagaggggcgaattatctcttggccttgctggtcgttatcctgcaagctatcactttattggcta
cggtgattggtagccgttctggtggttgtgatggtggtatgaaaaaagtcattttatctttggctctgggcacgtttggtttggg
gatggccgaatttggcattatgggcgtgctcacggagctggcgcataacg)

The temperature-sensitive pKD46-recApa plasmid was cured from the cell lines by growing at 37-42° C. Ampicillin sensitivity of the individual kanR colonies was also verified.

For host strains for antibiotic resistance plasmids (e.g. pUC replication origin; antibiotic selection; R6K replication origin; antibiotic selection) the kanR chromosomal marker was removed from ΔSbcDC::kanR using FRT recombination as described (Datsenko and Wanner, Supra, 2000). Briefly the ΔSbcDC::kanR cell line was transformed with pCP20 FRT plasmid (Datsenko and Wanner, Supra, 2000) and transformants grown at 30° C. and selected for ampicillin resistance. Individual colonies were streaked for single colonies on LB medium plates (without ampicillin) and grown at 43° C. to cure the temperature sensitive pCP20 plasmid. Single colonies on the 43° C. LB plate were streaked on LB amp and LB kan plates to verify loss of ampR pCP20 plasmid and kanR excision respectively. Individual amp and kan sensitive colonies were screened for ΔSbcDC by PCR using SbcDF and SbcCR primers (FIG. 1D). For the PCR product of the SbcDF primer and SbcCR primer, the size was 0.53 kb as shown in FIG. 1D (SEQ ID NO: 8).

For DH5α, the starting strain had the following genotype: F− φ80lacZΔM15 Δ(lacZYA-argF) U169 recA1 endA1 hsdR17 (rₖ−, mₖ+) gal-phoA supE44λ- thi-1 gyrA96 relA1. Following knockout of SbcCD and kanR excision, the knockout strain (DH5α [SbcCD-]) has the following genotype: F− φ80lacZAM15 Δ(lacZYA-argF) U169 recA1 endA1 hsdR17 (rₖ−, mₖ+) gal-phoA supE44λ- thi-1 gyrA96 relA1 ΔSbcDC.

An additional strain will be produced from DH5α [SbcCD-] by integrating a heat-inducible R6K rep protein cassette (att_{HK022}::pL (OL1-G to T) P42L-P106I-F107S P113S (P3−), SpecR StrepR) into the host genome as described in WO 2014/035457 to yield a new strain, DH5α R6K Rep [SbcCD−], which will have the genotype: DH5α att_{HK022}::pL (OL1-G to T) P42L-P106I-F107S P113S (P3−), SpecR StrepR; ΔSbcDC. This strain can be used for the production of plasmids having a R6K bacterial origin of replication.

R6K Replication Origin with RNA-OUT Selection. Additionally, NTC1050811 which has the genotype DH5α attx::P_{c}-RNA-IN-SacB, catR; att_{HK022}::pL (OL1-G to T) P42L-P106I-F107S P113S (P3−), SpecR StrepR; att_{φ80}::pARA-CI857ts, tetR as disclosed in WO 2019/183248 was also treated via the same method to knockout SbcDC but without kanR excision to yield NTC1300441 (DH5α ΔSbcDC) which has a genotype of DH5α att_{λ}::P_{c}-RNA-IN-SacB, catR; att_{HK022}::pL (OL1-G to T) P42L-P106I-F107S P113S (P3−), SpecR StrepR; att_{φ80}::pARA-CI857ts, tetR ΔSbcDC::kanR (SbcCD knockout copy cutter host strain derivative). NTC1050811-HF which is a derivative of NTC1050811 that includes a second copy of the RNA-IN-SacB expression cassette, without mutations in sbcB, recB, recD, recJ, uvrC and mcrA was also used to generate a knockout strain by the same method to yield NTC1050811-HF [SbcCD-] which does not have kanR excised.

pUC Replication Origin with RNA-OUT Selection. In addition NTC4862-HF, which is a derivative of NTC4862 as disclosed in WO 2008/153733 that includes a second copy of the RNA-IN-SacB expression cassette and which does not have mutations in sbcB, recB, recD, recJ, uviC and mcrA was used to generate a knockout strain by the same method to yield NTC4862-HF [SbcCD-] which does not have kanR excised.

### Example 2: SbcCD Knockout Strain Performance with Large Palindrome Vectors

SbcCD knockout strains were evaluated for their performance with large palindrome vectors, including evaluation of shake flask and HyperGRO production.

NTC1011641 (Genotype: Stbl4 att_{λ}::P_{c}-RNA-IN-SacB, catR; attH_{K022}::pL P42L-P106L-F107S (P3−) SpecR StrepR, as disclosed in WO 2019/183248) and NTC1300441 (Genotype: DH5α att_{λ}::P_{c}-RNA-IN-SacB, catR; att_{HK022}::pL (OL1-G to T) P42L-P106I-F107S P113S (P3−), SpecR StrepR; att_{φ80}::pARA-CI857ts, tetR ΔSbcDC::kanR) were transformed with the AAV vectors pAAV-GFP Nanoplasmid™ (pAAV-GFP NP) which includes a spacer region with an R6K bacterial replication origin and RNA-OUT selection as well as a palindromic AAV ITR and pAAV-GFP Mini Intronic Plasmid (pAAV-GFP MIP) which contains an intronic R6K bacterial replication origin and RNA-OUT selection as well as a 140 base pair inverted repeat with a 4 base pair intervening sequence.

Lu J, Williams J A, Luke J, Zhang F, Chu K, and Kay M A. 2017. Human Gene Therapy 28:125-34 disclose antibiotic free Mini-Intronic Plasmid (MIP) AAV vectors and suggest that MIP intron AAV vectors could have the vector backbone removed to create a short backbone AAV vector. Attempts to create a minicircle-like spacer region in Mini-Intronic Plasmid AAV vectors with intronic R6K origin and RNA-OUT selection marker (intronic Nanoplasmid vectors) were toxic presumably due to creation of a long 140 bp inverted repeat by such close juxtaposition of the AAV ITRs (e.g., pAAV-GFP MIP; see Table 2). By contrast, pAAV-GFP MIP was recoverable in a DH5α ΔSbcDC host strain and had excellent shake flask production yields (see Table 2). For each AAV ITR, the AAV ITR had a 26 bp palindromic sequence separated by 43 bp.

TABLE 2
DH5α SbcCD host strain enables viability of 140 bp inverted repeat vector

| | Spacing | | | | | Plasmid |
|---|---|---|---|---|---|---|
| | between ITRs | | Inverted | | Harvest | yield |
| AAV Vector | (bp) | | Repeat | Cell line | OD600 | (mg/L) |
| pAAV-GFP NP ^{a} | 492 | bp | AAV ITR | NTC1011641 | 4.1 | 13.1 |
| (corrected) (3.3 kb) | | | | (R6K SacB- | | |
| | | | | Stbl4) | | |
| pAAV-GFP NP ^{a} | 492 | bp | AAV ITR | NTC1300441 | 13.1 | 19.3 |
| (corrected) (3.3 kb) | | | | (DH5α ΔSbcDC) | | |
| pAAV-GFP MIP^{b} | 0 | bp | 140 bp | Toxic, | | |
| (3.0 kb) | | | inverted | unclonable in | | |
| | | | repeat | NTC1011641 | | |
| | | | | (R6K SacB-Stbl4) | | |
| pAAV-GFP MIP^{b} | 0 | bp | 140 bp | NTC1300441 | 13.3 | 24.3 |
| (3.0 kb) | | | inverted | (DH5α ΔSbcDC) | | |
| | | | repeat | | | |

Production conditions: 500 ml Plasmid + culture, 30° C. 12 hrs, shift to 37° C. for 8 hrs.
^{a} Nanoplasmid vector with spacer region R6K origin and RNA-OUT selection.
^{b}Nanoplasmid vector with intronic R6K origin and RNA-OUT selection.

This viability recovery in DH5α ΔSbcDC host strains is not limited to Nanoplasmid™ vectors. This is demonstrated by robust growth and HyperGRO plasmid production of a pUC origin kanR selection AAV helper plasmid containing an 85 bp inverted repeat with 17 base pairs intervening sequence in DH5α ΔSbcDC but not in DH5α (Table 3).

TABLE 3
HyperGRO fermentation production of fd6 inverted repeat derivative AAV helper

| | | | | Plasmid |
|---|---|---|---|---|
| | Inverted | | Harvest | yield |
| Plasmid | Repeat | Cell line | OD600 | (mg/L) |
| pUC-kanR Ad helper | 85 bp^{b} | DH5α ΔSbcDC | 118 ^{a} | 659 ^{a} |
| (19 kb) | | | | |
| pUC-kanR Ad helper | 85 bp^{b} | DH5α | NA, vector | NA, vector |
| (19 kb) | | | unclonable | unclonable |

^{a} 30° C., Shift to 42° C. at 55OD600, for 9 hr, 25° C. Hold
^{b}fd6 Ad helper vector and derivatives contain the 3′ Adenovirus terminal repeat and part of the adjacent 5′ Adenovirus terminal repeat creating an 85 bp inverted repeat with a short intervening loop

### Example 3: SbcCD Knockout Strain Performance with AAV ITR Vectors: ITR Stability and Shake Flask Production

The application of DH5α ΔSbcDC host strains to stabilize AAV ITR containing vectors was evaluated by next generation sequence confirmation of AAV vector transformed cell lines and production lots.

AAV ITRs are very difficult sequence using conventional sequencing (Doherty et al, Supra, 1993) but can be accurately sequenced using Next Generation Sequencing (Saveliev A Liu J, Li M, Hirata L, Latshaw C, Zhang J, Wilson J M. 2018. Accurate and rapid sequence analysis of Adeno-Associated virus plasmid by Illumina Next Generation Sequencing. Hum Gene Ther Methods 29:201-211).

To evaluate the DH5α ΔSbcDC host strains to stabilize AAV ITRs, nine different AAV ITR Nanoplasmid vectors from 2.4 to 5.4 kb were transformed into NTC 105081-HF [SbcCD−]. Individual colonies were screened for intact CTRs by SiaI digestion, then a single correct clone was submitted to Mass General Hospital (MGH) CCIB DNA Core (Cambridge Mass.) for Complete Plasmid Sequencing by Next Generation Sequencing. The results are summarized below in Table 4 and demonstrate ITR stability during transformation (25/26 screened colonies correct by SaI digest, of these 9/10 (one of each of the 9 Nanoplasmid vectors) are correct by Complete Plasmid Sequencing. ITR stability was maintained during production in shake flasks (5/5 preps correct by Complete Plasmid Sequencing). This demonstrates that the DH5α ΔSbcDC host strain stabilizes AAV ITRs during transformation and production.

TABLE 4
AAV ITR Nanoplasmid vector stability in NTC1050811-HF [SbcCD-]

| | | MGH Whole | MGH Whole |
|---|---|---|---|
| | SmaI restriction | plasmid Sequencing | plasmid Sequencing |
| | Digest Screen of | -transformed cell | -shake flask |
| Vector | transformed colonies | line | production lot |
| AAV NP 1 (4.4 kb) | (1/1 correct) | Correct | Correct |
| AAV NP 2 (4.8 kb) | (3/3 correct) | ITR | Correct |
| | | microdeletion | |
| | | Second clone | |
| | | correct | |
| AAV NP 3 (5.6 kb) | (1/1 correct) | Correct | Correct |
| AAV NP 4 (2.7 kb) | (4/4 correct) | Correct | Correct |
| AAV NP 5 (4.6 kb) | (1/1 correct) | Correct | Correct |
| AAV NP 6 (2.6 kb) | (4/4 correct) | Correct | Not Applicable |
| AAV NP 7 (2.6 kb) | (4/4 correct) | Correct | Not Applicable |
| AAV NP 8 (2.7 kb) | (3/4 correct) | Correct | Not Applicable |
| AAV NP 9 (2.4 kb) | (4/4 correct) | Correct | Not Applicable |
| Total | 25/26 correct | 9/10 correct | 5/5 correct |

Production conditions: 500 ml Plasmid + culture, 30° C. 12 hrs, shift to 37° C. for 8 hrs

The application of DH5α ΔSbcDC host strains to improve AAV ITR containing vector production was then evaluated with a standardized GFP AAV2 EGFP transgene vector, with different bacterial backbones either:
pUC origin- antibiotic selection AAV vector (Table 5);
pUC origin -RNA-OUT selection AAV vector (Table 6); or
R6K origin -RNA-OUT selection AAV Nanoplasmid vector (Table 7)

TABLE 5
pAAV-GFP (5.4 kb) (pUC origin, AmpR
selection) shake flask evaluation

| | | Plasmid | | |
|---|---|---|---|---|
| | Harvest | yield | Plasmid | ITR |
| Cell line | OD₆₀₀ | mg/L | quality | integrity |
| Stbl4 | 8 | 6.3 | Poor: smeared | ✓ |
| | | | monomer band | |
| DH5α | 14 | 6.4 | CCC monomer | ✓ |
| [SbcCD-] | | | | |

Production conditions: 500 mL Plasmid + Shake Flask Culture; 30 C. 12 hrs, shift to 37 C. for 8 hrs

TABLE 6
pAAV-GFP NTC8 (4.0 kb) (pUC origin, RNA-
OUT selection) shake flask evaluation

| | | Plasmid | | |
|---|---|---|---|---|
| | Harvest | yield | Plasmid | ITR |
| Cell line | OD₆₀₀ | mg/L | quality | integrity |
| NTC1011592 | 10 | 7 | CCC | ✓ |
| (Stbl4-SacB) | | | monomer | |
| NTC4862 HF | 11 | 6.5 | CCC | ✓ |
| [SbcCD-] | | | monomer | |

Production conditions: 500 mL Plasmid + Shake Flask Culture; 30 C. 12 hrs, shift to 37 C. for 8 hrs

TABLE 7
pAAV-GFP Nanoplasmid (3.3 kb) (R6K origin,
RNA-OUT selection) shake flask evaluation

| | | | Plasmid | | |
|---|---|---|---|---|---|
| | Production | Harvest | yield | Plasmid | ITR |
| Cell line | conditions^{b} | OD₆₀₀ | mg/L | quality | integrity |
| NTC1011641 | Flask A^{a} | 4 | 13.1 | CCC monomer | ✓ |
| (Stbl4) | | | | | |
| NTC1300441 | Flask A^{a} | 13 | 28.0 | CCC monomer | ✓ |
| (DH5α ΔSbcDC::kanR | | | | | |
| copy cutter) | | | | | |
| | Flask B^{a} | 8 | 12.3 | CCC monomer | ✓ |
| | (0.2% arabinose) | | | | |
| NTC1050811-HF | Flask A^{a} | 10 | 17.3 | CCC monomer | ✓ |
| [SbcCD-] | | | | | |
| (DH5α ΔSbcDC::kanR | | | | | |
| HF copy cutter) | | | | | |
| | Flask B^{a} | 7 | 8.1 | CCC monomer | ✓ |
| | (0.2% arabinose) | | | | |

^{a}Flask A contains 500 mL Plasmid +, 5 mLs 50% sucrose
Flask B contains 500 mL Plasmid +, 5 mLs 50% sucrose, 5 mLs 20% Arabinose
^{b}Production conditions: 30 C. 12 hrs, shift to 37 C. for 8 hrs

An additional panel of three larger 4.8-5.2 kb AAV Nanoplasmid vectors were evaluated in Stbl4 versus DH5α SbcCD NP host (Table 8). Dramatic yield and quality improvement were observed with the DH5α SbcCD host.

TABLE 8
AAV Nanoplasmid vector shake flask production
Stbl4 versus SbcCD NP host comparison

| | | | | Plasmid | |
|---|---|---|---|---|---|
| | | | Harvest | yield | Plasmid |
| Vector | Cell line^{b} | Production culture | OD600 ^{a} | mg/mL ^{a} | quality ^{a} |
| AAV | NTC1011641 | 30° C. 12 h, shift to | 2.44 | 4.9 | Poor: smeared |
| Nanoplasmid 1 | Stbl4 | 37° C. 8 h | | | monomer band |
| (5.0 kb) | | | | | |
| AAV | NTC1300441 | 30° C. 12 h, shift to | 12.84 | 25.7 | CCC monomer |
| Nanoplasmid 1 | DH5α SbcDC | 37° C. 8 h + 0.2% | | | |
| (5.0 kb) | | arabinose | | | |
| AAV | NTC1011641 | 30° C. 12 h, shift to | 1.36 | 0.9 | Poor: smeared |
| Nanoplasmid 2 | Stbl4 | 37° C. 8 h | | | monomer band |
| (5.2 kb) | | | | | |
| AAV | NTC1300441 | 30° C. 12 h, shift to | 12.66 | 40.0 | CCC monomer |
| Nanoplasmid 2 | DH5α SbcDC | 37° C. 8 h + 0.2% | | | |
| (5.2 kb) | | arabinose | | | |
| AAV | NTC1011641 | 30° C. 12 h, shift to | 11.1 | 17.7 | Poor: smeared |
| Nanoplasmid 3 | Stbl4 | 37° C. 8 h | | | monomer band |
| (4.8 kb) | | | | | |
| AAV | NTC1300441 | 30° C. 12 h, shift to | 11.16 | 25.2 | CCC monomer |
| Nanoplasmid 3 | DH5α SbcDC | 37° C. 8 h + 0.2% | | | |
| (4.8 kb) | | arabinose | | | |

^{a} 500 mL Plasmid + Shake Flask Culture

Summary: The DH5α SbcCD host showed improved plasmid production and/or plasmid quality compared to the Stbl4 host with AAV ITR vectors, especially with larger therapeutic transgene encoding AAV ITR vectors (Table 8).

### Example 4: SbcCD Knockout Strain Performance with AAV ITR Vectors: HyperGRO Fermentation

The application of DH5α ΔSbcDC host strains to improve AAV ITR containing vector production was then evaluated in HyperGRO fermentation with: the 3.3 kb AAV2 EGFP transgene R6K origin-RNA-OUT marker Nanoplasmid vector pAAV-GFP Nanoplasmid (evaluated in shake flask in Example 3) in DH5α ΔSbcDC Nanoplasmid host compared to Stbl4 Nanoplasmid host; and a 12 kb pUC origin-kanR AAV vector in DH5α ΔSbcDC compared to Stbl3. The results are summarized in Tables 9 and 10.

TABLE 9
pAAV-GFP Nanoplasmid (3.3 kb) (R6K origin, RNA-
OUT selection) HyperGRO fermentation evaluation

| | HyperGRO | | Plasmid | | |
|---|---|---|---|---|---|
| | Ferm | Harvest | yield | Plasmid | ITR |
| Cell line | conditions | OD₆₀₀ | mg/L | quality | integrity |
| NTC1011641 | ^{a} | 71 | 260 | Poor, multiple | ✓ |
| (Stbl4) | | | | species | |
| NTC1300441 | ^{b} | 133 | 215 | CCC monomer | ✓ |
| (DH5α ΔSbcDC::kanR | | | | | |
| copy cutter) | | | | | |
| NTC1050811-HF | ^{b} | 157 | 387 | CCC monomer | ✓ |
| [SbcCD-] | | | | | |
| (DH5α ΔSbcDC::kanR | | | | | |
| HF copy cutter) | | | | | |

^{a} 30° C., Shift to 42° C. at 55OD600, for 9 hr, 25° C. Hold
^{b} 30° C., Shift to 42° C. at 55OD600, for 9 hr, 25° C. Hold; 0.2% Arabinose in medium

TABLE 10
pAAV vector (12 kb pUC origin-kanR) HyperGRO fermentation evaluation

| | HyperGRO | | Plasmid | | |
|---|---|---|---|---|---|
| | Ferm | Harvest | yield | Plasmid | ITR |
| Cell line | conditions | OD₆₀₀ | mg/L | quality | integrity |
| Stbl3 | ^{a} | 20 | 171 | CCC monomer | ✓ |
| | ^{b} | 27 | 214 | | |
| | ^{c} | 25 | 152 | | |
| DH5α [SbcCD-] | ^{d} | 93 | 895 | CCC monomer | ✓ |

^{a} 30° C., Shift to 42° C. at 55OD600, for 9 hr, 25° C. Hold
^{b} 30->37° C. ramp 24-36 h
^{c} 30° C., Shift to 37° C. at 55OD600 until OD drops or lysis, 25° C. Hold
^{d} 30° C., Shift to 37° C. at 30 h until OD drops or lysis, 25° C. Hold

Summary: The DH5α SbcCD host showed improved plasmid production and/or plasmid quality compared to the Stbl3 or Stbl4 host with AAV ITR vectors, especially with larger therapeutic transgene encoding AAV ITR vectors (Table 10).

### Example 5: SbcCD Knockout Strain Performance with Non-Palindrome Containing Vectors

DH5α [SbcCD−] was evaluated versus DH5α for production yield of a standard vector (12 kb pHelper vector, pUC origin-kanR selection). The results indicated that DH5α [SbcCD-] is superior to DH5α for production of standard plasmids.

TABLE 11
pHelper vector (12 kb pUC origin-kanR)
HyperGRO fermentation evaluation

| | | plasmid |
|---|---|---|
| | | yield |
| Plasmid | Harvest OD600 | mg/L |
| pHelper-KanR (DH5α) | 94 | 762 |
| pHelper-KanR (DH5α [SbcCD-]) | 111 | 1230 |

Production conditions: 30° C., Shift to 42° C. at 55OD600, for 9 hr, 25° C. Hold

This was unexpected since while SbcCD knockout can stabilize palindromes, it would not be expected improve yield of standard plasmids that do not contain palindromes.

### Example 6: Improved Plasmid PolyA Repeat Stability in DH5α [SbcCD−] Compared to Stbl4

A pUC-AmpR plasmid vector encoding a A90 repeat was transformed into Stbl4 or DH5α [SbcCD−] and the stability of the A90 repeat in 4 individual colonies from each transformation were determined by sequencing. All 4 of the Stbl4 colonies had deleted at least 20 bps of the A90 repeat (i.e. all 4 colonies were <A70) while all 4 of the DH5α [SbcCD−] colonies were >A70 and 2/4 had intact A90 repeats. This demonstrates DH5α [SbcCD−] stabilizes simple sequence repeats compared to a stabilizing host in the art. This was unexpected since SbcCD knockout would not be expected to stabilize simple repeats.

Plasmid vectors encoding an A117 repeat were transformed into DH5α [SbcCD-] and NTC1050811-HF [SbcCD-] and the stability of the A117 repeat was determined by sequencing. The cells were cultured at 30° C. for 12 hours and ramped to 37° C. at 24 EFT until the OD dropped or lysis was observed, after which the cells were held at 25° C., under HyperGro conditions as in Example 4. All of the transformed cells lines (2 DH5α [SbcCD-], 2 NTC1050811-HF [SbcCD-]) had intact A117 repeats and high yield as shown in Table 12 below. This was unexpected since SbcCD knockout would not be expected to stabilize simple repeats.

TABLE 12
A117 Repeat stability and production in engineered E. coli host cells

| | | | | | | Ferm |
|---|---|---|---|---|---|---|
| | | | | Plasmid | | harvest |
| | | Biomass | Plasmid | specific | Plasmid | polyA |
| | | yield | yield | yield | Quality | Sequence |
| Vector | Host strain | (OD600) | (mg/L) | (mg/L/OD600) | (AGE) | (Sanger) |
| 7318 bp | DH5α | 176 | 940 | 5.3 | CCC | A117 |
| kanR A117 | [SbcCD-] | | | | | |
| 7867 bp | DH5α | 172 | 702 | 4.1 | CCC | A117 |
| kanR A117 | [SbcCD-] | | | | | |
| 5262 bp | NTC1050811-HF | 124 | 740 | 6.0 | CCC | A117 |
| RNA-OUT A117 | [SbcCD-] | | | | | |
| 5811 bp | NTC1050811-HF | 118 | 1007 | 8.5 | CCC | A117 |
| RNA-OUT A117 | [SbcCD-] | | | | | |

The same procedure was used in DH5α [SbcCD-], NTC4862-HF [SbcCD-] and NTC 1050811-HF [SbcCD-] for plasmid vectors encoding A98-100 and A99-100 repeats. All of the transformed cell lines had intact repeats. All of the transformed cell lines had intact repeats and high yield. This was unexpected since SbcCD knockout would not be expected to stabilize simple repeats.

TABLE 13
polyA Repeat stability and production in engineered E. coli host cells

| | | | | | | Ferm |
|---|---|---|---|---|---|---|
| | | | | Plasmid | | harvest |
| | | Biomass | Plasmid | specific | Plasmid | polyA |
| | | yield | yield | yield | Quality | Sequence |
| Vector | Host strain | (OD600) | (mg/L) | (mg/L/OD600) | (AGE) | (Sanger) |
| polyA98-100 | DH5α | 139 | 1143 | 8.2 | CCC | A98-99 |
| (6560 bp) | [SbcCD-] | | | | | |
| <kanR pUC> | | | | | | |
| polyA98-100 | NTC4862-HF | 71 | 677 | 9.5 | CCC | A98-100 |
| (5787 bp) | [SbcCD-] | | | | | |
| <RNAOUT pUC> | | | | | | |
| (4755 bp) | NTC1050811-HF | 120 | 747 | 6.2 | CCC | A98-99 |
| polyA99-100 | [SbcCD-] | | | | | |
| <RNAOUT R6K> | | | | | | |
| (4755 bp) | NTC1050811-HF | 93 | 632 | 6.8 | CCC | A99-100 |
| polyA99-100 | [SbcCD-] | | | | | |
| RNAOUT> R6K> | | | | | | |
| (4757 bp) | NTC1050811-HF | 94 | 638 | 6.8 | CCC | A99-100 |
| polyA99-100 | [SbcCD-] | | | | | |
| R6K> RNAOUT> | | | | | | |

### Example 7: Additional Cell Lines

The foregoing examples may be repeated using DH1, JM107, JM108, JM109, MG1655, XL1Blue and like cell lines and may use SURE, SURE2, Stbl2, Stbl3, Stbl4 and non-SbcC, SbcD and/or SbcCD knockout strains.

All references, including publications, patent applications, and patents, cited herein are hereby incorporated by reference to the same extent as if each reference were individually and specifically indicated to be incorporated by reference and were set forth in its entirety herein.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An engineered Escherichia coli (E. coli) host cell, wherein the engineered E. coli host cell comprises a gene knockout of at least one gene selected from the group consisting of SbcC and SbcD, and wherein the engineered E. coli host cell comprises a sbcB gene, a recB gene, a recD gene, and a recJ gene, and wherein there are no engineered viability- or yield-reducing mutations in any of the sbcB, recB, recD, and recJ genes.

2-15. (canceled)

16. The engineered E. coli host cell of claim 1, wherein the engineered E. coli host cell further comprises a genomic nucleic acid sequence encoding a Rep protein, wherein the Rep protein comprises an amino acid sequence of at least 90% sequence identity to a sequence selected from the group consisting of SEQ ID NO: 39, SEQ ID NO: 40, SEQ ID NO: 41, SEQ ID NO: 42, SEQ ID NO: 34, and SEQ ID NO: 35.

17-18. (canceled)

19. The engineered E. coli host cell of claim 1, further comprising a genomic nucleic acid sequence encoding a temperature-sensitive lambda repressor.

20. The engineered E. coli host cell of claim 19, wherein the temperature-sensitive lambda repressor is cITs857.

22. The engineered E. coli host cell of claim 19, wherein the temperature-sensitive lambda repressor comprises an amino acid sequence with at least 90% sequence identity to SEQ ID NO: 37.

24. The engineered E. coli host cell of claim 19, wherein the temperature-sensitive lambda repressor is a phage φ80 attachment site chromosomally integrated copy of a arabinose inducible CITs857 gene.

25-38. (canceled)

39. The engineered E. coli host cell of claim 1, wherein the engineered E. coli host cell does not include any engineered viability- or yield-reducing mutations in at least one of uvrC, mcrA, and mcrBC-hsd-mrr.

40-41. (canceled)

42. The engineered E. coli host cell of claim 1, wherein sbcB gene comprises a sequence having at least 90% sequence identity to SEQ ID NO: 11, wherein the recB gene comprises a sequence having at least 90% sequence identity to SEQ ID NO: 12, wherein the recD gene comprises a sequence having at least 90% sequence identity to SEQ ID NO: 13, and wherein the recJ gene comprises a sequence having at least 90% sequence identity to SEQ ID NO: 65.

43-45. (canceled)

46. The engineered E. coli host cell of claim 1, further comprising a vector, wherein the vector comprises a nucleic acid sequence having an inverted repeat, a direct repeat, or a palindrome.

47-51. (canceled)

52. The engineered E. coli host cell of claim 1, further comprising a vector, wherein the vector is an AAV vector, a lentiviral vector, a retroviral vector, or a mRNA vector containing a polyA repeat.

53-55. (canceled)

56. The engineered E. coli host cell of claim 1, further comprising a plasmid vector.

57-65. (canceled)

66. The engineered E. coli host cell of claim 56, wherein the plasmid vector is a eukaryotic pUC-free minicircle expression vector that comprises: (i) a eukaryotic region sequence encoding a gene of interest and having 5′ and 3′ ends; and (ii) a spacer region having a length of less than 1000 basepairs that links the 5′ and 3′ ends of the eukaryotic region sequence and that comprises a R6K bacterial replication origin and a RNA selectable marker.

67. (canceled)

68. The engineered E. coli host cell of claim 66, wherein the gene of interest comprises a structured DNA sequence selected from the group consisting of an inverted repeat sequence, a direct repeat sequence, a homopolymeric repeat sequence, an eukaryotic origin of replication, a polyA repeat, a SV40 origin of replication, a viral LTR, a Lentiviral LTR, a Retroviral LTR, a transposon IR/DR repeat, a Sleeping Beauty transposon IR/DR repeat, and an AAV ITR.

69-72. (canceled)

73. A method for producing an engineered Escherichia coli (E. coli) cell, comprising:
knocking out at least one gene selected from the group consisting of SbcC and SbcD in a starting E. coli cell to yield the engineered E. coli cell, wherein the starting E. coli cell comprises a sbcB gene, a recB gene, a recD gene, and a recJ gene, and wherein there are no engineered viability- or yield-reducing mutations in any of the sbcB, recB, recD, and recJ genes in the engineered E. coli cell.

74-76. (canceled)

77. The method of claim 73, wherein the starting E. coli cell does not include an engineered viability- or yield-reducing mutation in at least one of uvrC, mcrA, mcrBC-hsd-mrr, and combinations thereof.

78-90. (canceled)

91. A method for improved vector production, comprising:
providing an engineered Escherichia coli (E. coli) host cell comprising a gene knockout of at least one gene selected from the group consisting of SbcC, SbcD and SbcCD, and wherein the engineered E coli host cell comprises a sbcB gene, a recB gene, a recD gene, and a recJ gene, and wherein there are no viability- or yield-reducing mutations in any of the sbcB, recB, recD, and recJ genes, and wherein the engineered Escherichia coli (E. coli) host cell comprises a vector;
incubating the engineered E. coli host cell under conditions sufficient to replicate the vector.

92-93. (canceled)

94. The method of claim 91, wherein the step of incubating the transfected host cell under conditions sufficient to replicate the vector is performed by a fed-batch fermentation, wherein the fed-batch fermentation comprises growing the transfected host cells at a first temperature of about 25° C. to about 32° C. during a first portion of the fed-batch phase, followed by a temperature up-shift to a second temperature of about 37° C. to about 42° C. during a second portion of the fed-batch phase.

95-100. (canceled)

101. The engineered E. coli host cell of claim 1, further comprising a gene selected from the group consisting of fhuA2 and glnV.

102. The engineered E. coli host cell of claim 1, further comprising a fhuA2 gene and a glnV gene.

103. The engineered E. coli host cell of claim 1, further comprising a gene knockout of a dcm gene.

104. The engineered E. coli host cell of claim 1, wherein the host cell does not contain a supE44 gene.

105. The engineered E. coli host cell of claim 1, further comprising a fhuA2 gene and a glnV gene, and wherein the host cell does not contain a supE44 gene.


SEQUENCE LISTING (65 sequences)

SEQ ID NO: 1 - DNA, 70 bp, synthetic construct
  misc_feature 1..70: note = Description of Artificial Sequence: Syntheticprimer
  source 1..70: mol_type = other DNA
ccctctgtattcattatcctgctgaatagttatttcactgcaaacgtactcatatgaatatcctccttag

SEQ ID NO: 2 - DNA, 70 bp, synthetic construct
  misc_feature 1..70: note = Description of Artificial Sequence: Syntheticprimer
  source 1..70: mol_type = other DNA
tctgtttgggtataatcgcgcccatgctttttcgccagggaaccgttatgtgtaggctggagctgcttcg

SEQ ID NO: 3 - DNA, 22 bp, synthetic construct
  misc_feature 1..22: note = Description of Artificial Sequence: Syntheticprimer
  source 1..22: mol_type = other DNA
cgtctcgccatgatttgccctg

SEQ ID NO: 4 - DNA, 22 bp, synthetic construct
  misc_feature 1..22: note = Description of Artificial Sequence: Syntheticprimer
  source 1..22: mol_type = other DNA
cgttatgcgccagctccgtgag

SEQ ID NO: 5 - DNA, 1576 bp, synthetic construct
  misc_feature 1..1576: note = Description of Artificial Sequence: Syntheticpolynucleotide
  source 1..1576: mol_type = other DNA
tctgtttgggtataatcgcgcccatgctttttcgccagggaaccgttatgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatgagtacgtttgcagtgaaataactattcagcaggataatgaatacagaggg

SEQ ID NO: 6 - DNA, 5403 bp, synthetic construct
  misc_feature 1..5403: note = Description of Artificial Sequence: Syntheticpolynucleotide
  source 1..5403: mol_type = other DNA
cgtctcgccatgatttgccctgttgtaataaataggttgcgatcattaatgcgacgtcattatgcgtcagatttatgacagatttatgaaaagctcgtcgcacatatcttcaggttattgatttccgtggcgcagaaaaaagcaaatggcacatctgtttgggtataatcgcgcccatgctttttcgccagggaaccgttatgcgcatccttcacacctcagactggcatctcggccagaacttctacagtaaaagccgcgaagctgaacatcaggcttttcttgactggctgctggagacagcacaaacccatcaggtggatgcgattattgttgccggtgatgttttcgataccggctcgccgcccagttacgcccgcacgttatacaaccgttttgttgtcaatttacagcaaactggctgtcatctggtggtactggcaggaaaccatgactcggtcgccacgctgaatgaatcgcgcgatatcatggcgttcctcaatactaccgtggtcgccagcgccggacatgcgccgcaaatcttgcctcgtcgcgacgggacgccaggcgcagtgctgtgccccattccgtttttacgtccgcgtgacattattaccagccaggcggggcttaacggtattgaaaaacagcagcatttactggcagcgattaccgattattaccaacaacactatgccgatgcctgcaaactgcgcggcgatcagcctctgcccatcatcgccacgggacatttaacgaccgtgggggccagtaaaagtgacgccgtgcgtgacatttatattggcacgctggacgcgtttccggcacaaaactttccaccagccgactacatcgcgctcgggcatattcaccgcgcacagattattggcggcatggaacatgttcgctattgcggctcccccattccactgagttttgatgaatgcggtaagagtaaatatgtccatctggtgacattttcaaacggcaaattagagagcgtggaaaacctgaacgtaccggtaacgcaacccatggcagtgctgaaaggcgatctggcgtcgattaccgcacagctggaacagtggcgcgatgtatcgcaggagccacctgtctggctggatatcgaaatcactactgatgagtatctgcatgatattcagcgcaaaatccaggcattaaccgaatcattgcctgtcgaagtattgctggtacgtcggagtcgtgaacagcgcgagcgtgtgttagccagccaacagcgtgaaaccctcagcgaactcagcgtcgaagaggtgttcaatcgccgtctggcactggaagaactggatgaatcgcagcagcaacgtctgcagcatcttttcaccacgacgttgcataccctcgccggagaacacgaagcatgaaaattctcagcctgcgcctgaaaaacctgaactcattaaaaggcgaatggaagattgatttcacccgcgagccgttcgccagcaacgggctgtttgctattaccggcccaacaggtgcggggaaaaccaccctgctggacgccatttgtctggcgctgtatcacgaaactccgcgtctctctaacgtttcacaatcgcaaaatgatctcatgacccgcgataccgccgaatgtctggcggaggtggagtttgaagtgaaaggtgaagcgtaccgtgcattctggagccagaatcgggcgcgtaaccaacccgacggtaatttgcaggtgccacgcgtagagctggcgcgctgcgccgacggcaaaattctcgccgacaaagtgaaagataagctggaactgacagcgacgttaaccgggctggattacgggcgcttcacccgttcgatgctgctttcgcaggggcaatttgctgccttcctgaatgccaaacccaaagaacgcgcggaattgctcgaggagttaaccggcactgaaatctacgggcaaatctcggcgatggtttttgagcagcacaaatcggcccgcacagagctggagaagctgcaagcgcaggccagcggcgtcacgttgctcacgccggaacaagtgcaatcgctgacagcgagtttgcaggtacttactgacgaagaaaaacagttaattaccgcgcagcagcaagaacaacaatcgctaaactggttaacgcgtcaggacgaattgcagcaagaagccagccgccgtcagcaggccttgcaacaggcgttagccgaagaagaaaaagcgcaacctcaactggcggcgcttagtctggcacaaccggcacgaaatcttcgtccacactgggaacgcatcgcagaacacagcgcggcgctggcgcatattcgccagcagattgaagaagtaaatactcgcttacagagcacaatggcgcttcgcgcgagcattcgccaccacgcggcgaagcagtcagcagaattacagcagcagcaacaaagcctgaatacctggttacaggaacacgaccgcttccgtcagtggaacaacgaaccggcgggttggcgtgcgcagttctcccaacaaaccagcgatcgcgagcatctgcggcaatggcagcaacagttaacccatgctgagcaaaaacttaatgcgcttgcggcgatcacgttgacgttaaccgccgatgaagttgctaccgccctggcgcaacatgctgagcaacgcccactgcgtcagcacctggtcgcgctgcatggacagattgttccccaacaaaaacgtctggcgcagttacaggtcgctatccagaatgtcacgcaagaacagacgcaacgtaacgccgcacttaacgaaatgcgccagcgttataaagaaaagacgcagcaacttgccgatgtgaaaaccatttgcgagcaggaagcgcgcatcaaaacgctggaagctcaacgtgcacagttacaggcgggtcagccttgcccactttgtggttccaccagccacccggcggtcgaggcgtatcaggcgctggagcctggcgttaatcagtctcgattactggcgctggaaaacgaagttaaaaagctcggtgaagaaggtgcgacgctacgtgggcaactggacgccataacaaagcagcttcagcgtgatgaaaacgaagcgcaaagcctccgacaagatgagcaagcacttactcaacaatggcaagccgtcacggccagcctcaatatcaccttgcagccactggacgatattcaaccgtggctggatgcacaagatgagcacgaacgccagctgcggttactcagccaacggcatgaattacaagggcagattgccgcgcataatcagcaaattatccagtatcaacagcaaattgaacaacgccagcaactacttttaacgacattgacgggttatgcactgacattgccacaggaagatgaagaagagagctggttggcgacacgtcagcaagaagcgcagagctggcagcaacgccagaacgaattaaccgcgctgcaaaaccgtattcagcagctgacgccgattctggaaacgttgccgcaaagtgatgaactcccgcactgcgaagaaactgtggtattggaaaactggcggcaggtacatgaacaatgtctcgcattacacagccagcagcagacgttacagcaacaggatgttctggcggcgcaaagtctgcaaaaagcccaggcgcagtttgacaccgcgctacaggccagcgtctttgacgatcagcaggcgttccttgcggcgctaatggatgaacaaacactaacgcagctggaacagctcaagcagaatctggaaaaccagcgccgtcaggcgcaaactctggtcactcagacagcagaaacgctggcacagcatcaacaacaccgacctgacgacgggttggctctcactgtgacggtggagcagattcagcaagagttagcgcaaactcaccaaaagttgcgtgaaaacaccacgagtcaaggcgagattcgccagcagctgaagcaggatgcagataaccgtcagcaacaacaaaccttaatgcagcaaattgctcaaatgacgcagcaggttgaggactggggatatctgaattcgctaataggttccaaagagggcgataaattccgcaagtttgcccaggggctgacgctggataatttagtccatctcgctaatcagcaacttacccggctgcacgggcgctatctgttacagcgcaaagccagcgaggcgctggaagtcgaggttgttgatacctggcaggcagatgcggtacgcgatacccgtaccctttccggcggcgaaagtttcctcgttagtctggcgctggcgctggcgctttcggatctggtcagccataaaacacgtattgactcgctgttccttgatgaaggttttggcacgctggatagcgaaacgctggataccgcccttgatgcgctggatgccctgaacgccagtggcaaaaccatcggtgtgattagccacgtagaagcgatgaaagagcgtattccggtgcagatcaaagtgaaaaagatcaacggcctgggctacagcaaactggaaagtacgtttgcagtgaaataactattcagcaggataatgaatacagaggggcgaattatctcttggccttgctggtcgttatcctgcaagctatcactttattggctacggtgattggtagccgttctggtggttgtgatggtggtatgaaaaaagtcattttatctttggctctgggcacgtttggtttggggatggccgaatttggcattatgggcgtgctcacggagctggcgcataacgtaggaatttcgattcctgccgccgggcatatgatctcgtattatgcactgggggtggtggtcggtgcgccaatcatcgcactcttttccagccgctactcactcaaacatatcttgttgtttctggtggcgttgtgcgtcattggcaacgccatgttcacgctctcttcgtcttacctgatgctcgccattggtcggctggtatccggctttccgcatggcgcattttttggcgtcggagcgatcgtgttatcaaaaattatcaaacccggaaaagtcaccgccgccgtggcggggatggtttccgggatgacagtcgccaatttgctgggcattccgctgggaacgtatttaagtcaggaatttagctggcgttacacctttttattgatcgctgtttttaatattgcggtgatggcatcggtctatttttgggtgccagatattcgcgacgaggcgaaaggaaatctgcgcgaacaatttcactttttgcgcagcccggccccgtggttaattttcgccgccacgatgtttggcaacgcaggtgtgtttgcctggttcagctacgtaaagccatacatgatgtttatttccggtttttcggaaacggcgatgacctttattatgatgttagtt

SEQ ID NO: 7 - DNA, 1922 bp, synthetic construct
  misc_feature 1..1922: note = Description of Artificial Sequence: Syntheticpolynucleotide
  source 1..1922: mol_type = other DNA
cgtctcgccatgatttgccctgttgtaataaataggttgcgatcattaatgcgacgtcattatgcgtcagatttatgacagatttatgaaaagctcgtcgcacatatcttcaggttattgatttccgtggcgcagaaaaaagcaaatggcacatctgtttgggtataatcgcgcccatgctttttcgccagggaaccgttatgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatgagtacgtttgcagtgaaataactattcagcaggataatgaatacagaggggcgaattatctcttggccttgctggtcgttatcctgcaagctatcactttattggctacggtgattggtagccgttctggtggttgtgatggtggtatgaaaaaagtcattttatctttggctctgggcacgtttggtttggggatggccgaatttggcattatgggcgtgctcacggagctggcgcataacg

SEQ ID NO: 8 - DNA, 529 bp, synthetic construct
  misc_feature 1..529: note = Description of Artificial Sequence: Syntheticpolynucleotide
  source 1..529: mol_type = other DNA
cgtctcgccatgatttgccctgttgtaataaataggttgcgatcattaatgcgacgtcattatgcgtcagatttatgacagatttatgaaaagctcgtcgcacatatcttcaggttattgatttccgtggcgcagaaaaaagcaaatggcacatctgtttgggtataatcgcgcccatgctttttcgccagggaaccgttatgtgtaggctggagctgcttcgaagttcctatactttctagagaataggaacttcggaataggaactaaggaggatattcatatgagtacgtttgcagtgaaataactattcagcaggataatgaatacagaggggcgaattatctcttggccttgctggtcgttatcctgcaagctatcactttattggctacggtgattggtagccgttctggtggttgtgatggtggtatgaaaaaagtcattttatctttggctctgggcacgtttggtttggggatggccgaatttggcattatgggcgtgctcacggagctggcgcataacg

SEQ ID NO: 9 - DNA, 3147 bp, Escherichia coli
  source 1..3147: mol_type = genomic DNA
atgaaaattctcagcctgcgcctgaaaaacctgaactcattaaaaggcgaatggaagattgatttcacccgcgagccgttcgccagcaacgggctgtttgctattaccggcccaacaggtgcggggaaaaccaccctgctggacgccatttgtctggcgctgtatcacgaaactccgcgtctctctaacgtttcacaatcgcaaaatgatctcatgacccgcgataccgccgaatgtctggcggaggtggagtttgaagtgaaaggtgaagcgtaccgtgcattctggagccagaatcgggcgcgtaaccaacccgacggtaatttgcaggtgccacgcgtagagctggcgcgctgcgccgacggcaaaattctcgccgacaaagtgaaagataagctggaactgacagcgacgttaaccgggctggattacgggcgcttcacccgttcgatgctgctttcgcaggggcaatttgctgccttcctgaatgccaaacccaaagaacgcgcggaattgctcgaggagttaaccggcactgaaatctacgggcaaatctcggcgatggtttttgagcagcacaaatcggcccgcacagagctggagaagctgcaagcgcaggccagcggcgtcacgttgctcacgccggaacaagtgcaatcgctgacagcgagtttgcaggtacttactgacgaagaaaaacagttaattaccgcgcagcagcaagaacaacaatcgctaaactggttaacgcgtcaggacgaattgcagcaagaagccagccgccgtcagcaggccttgcaacaggcgttagccgaagaagaaaaagcgcaacctcaactggcggcgcttagtctggcacaaccggcacgaaatcttcgtccacactgggaacgcatcgcagaacacagcgcggcgctggcgcatattcgccagcagattgaagaagtaaatactcgcttacagagcacaatggcgcttcgcgcgagcattcgccaccacgcggcgaagcagtcagcagaattacagcagcagcaacaaagcctgaatacctggttacaggaacacgaccgcttccgtcagtggaacaacgaaccggcgggttggcgtgcgcagttctcccaacaaaccagcgatcgcgagcatctgcggcaatggcagcaacagttaacccatgctgagcaaaaacttaatgcgcttgcggcgatcacgttgacgttaaccgccgatgaagttgctaccgccctggcgcaacatgctgagcaacgcccactgcgtcagcacctggtcgcgctgcatggacagattgttccccaacaaaaacgtctggcgcagttacaggtcgctatccagaatgtcacgcaagaacagacgcaacgtaacgccgcacttaacgaaatgcgccagcgttataaagaaaagacgcagcaacttgccgatgtgaaaaccatttgcgagcaggaagcgcgcatcaaaacgctggaagctcaacgtgcacagttacaggcgggtcagccttgcccactttgtggttccaccagccacccggcggtcgaggcgtatcaggcgctggagcctggcgttaatcagtctcgattactggcgctggaaaacgaagttaaaaagctcggtgaagaaggtgcgacgctacgtgggcaactggacgccataacaaagcagcttcagcgtgatgaaaacgaagcgcaaagcctccgacaagatgagcaagcacttactcaacaatggcaagccgtcacggccagcctcaatatcaccttgcagccactggacgatattcaaccgtggctggatgcacaagatgagcacgaacgccagctgcggttactcagccaacggcatgaattacaagggcagattgccgcgcataatcagcaaattatccagtatcaacagcaaattgaacaacgccagcaactacttttaacgacattgacgggttatgcactgacattgccacaggaagatgaagaagagagctggttggcgacacgtcagcaagaagcgcagagctggcagcaacgccagaacgaattaaccgcgctgcaaaaccgtattcagcagctgacgccgattctggaaacgttgccgcaaagtgatgaactcccgcactgcgaagaaactgtggtattggaaaactggcggcaggtacatgaacaatgtctcgcattacacagccagcagcagacgttacagcaacaggatgttctggcggcgcaaagtctgcaaaaagcccaggcgcagtttgacaccgcgctacaggccagcgtctttgacgatcagcaggcgttccttgcggcgctaatggatgaacaaacactaacgcagctggaacagctcaagcagaatctggaaaaccagcgccgtcaggcgcaaactctggtcactcagacagcagaaacgctggcacagcatcaacaacaccgacctgacgacgggttggctctcactgtgacggtggagcagattcagcaagagttagcgcaaactcaccaaaagttgcgtgaaaacaccacgagtcaaggcgagattcgccagcagctgaagcaggatgcagataaccgtcagcaacaacaaaccttaatgcagcaaattgctcaaatgacgcagcaggttgaggactggggatatctgaattcgctaataggttccaaagagggcgataaattccgcaagtttgcccaggggctgacgctggataatttagtccatctcgctaatcagcaacttacccggctgcacgggcgctatctgttacagcgcaaagccagcgaggcgctggaagtcgaggttgttgatacctggcaggcagatgcggtacgcgatacccgtaccctttccggcggcgaaagtttcctcgttagtctggcgctggcgctggcgctttcggatctggtcagccataaaacacgtattgactcgctgttccttgatgaaggttttggcacgctggatagcgaaacgctggataccgcccttgatgcgctggatgccctgaacgccagtggcaaaaccatcggtgtgattagccacgtagaagcgatgaaagagcgtattccggtgcagatcaaagtgaaaaagatcaacggcctgggctacagcaaactggaaagtacgtttgcagtgaaataa

SEQ ID NO: 10 - DNA, 1227 bp, Escherichia coli
  source 1..1227: mol_type = genomic DNA
atgctttttcgccagggaaccgttatgcgcatccttcacacctcagactggcatctcggccagaacttctacagtaaaagccgcgaagctgaacatcaggcttttcttgactggctgctggagacagcacaaacccatcaggtggatgcgattattgttgccggtgatgttttcgataccggctcgccgcccagttacgcccgcacgttatacaaccgttttgttgtcaatttacagcaaactggctgtcatctggtggtactggcaggaaaccatgactcggtcgccacgctgaatgaatcgcgcgatatcatggcgttcctcaatactaccgtggtcgccagcgccggacatgcgccgcaaatcttgcctcgtcgcgacgggacgccaggcgcagtgctgtgccccattccgtttttacgtccgcgtgacattattaccagccaggcggggcttaacggtattgaaaaacagcagcatttactggcagcgattaccgattattaccaacaacactatgccgatgcctgcaaactgcgcggcgatcagcctctgcccatcatcgccacgggacatttaacgaccgtgggggccagtaaaagtgacgccgtgcgtgacatttatattggcacgctggacgcgtttccggcacaaaactttccaccagccgactacatcgcgctcgggcatattcaccgcgcacagattattggcggcatggaacatgttcgctattgcggctcccccattccactgagttttgatgaatgcggtaagagtaaatatgtccatctggtgacattttcaaacggcaaattagagagcgtggaaaacctgaacgtaccggtaacgcaacccatggcagtgctgaaaggcgatctggcgtcgattaccgcacagctggaacagtggcgcgatgtatcgcaggagccacctgtctggctggatatcgaaatcactactgatgagtatctgcatgatattcagcgcaaaatccaggcattaaccgaatcattgcctgtcgaagtattgctggtacgtcggagtcgtgaacagcgcgagcgtgtgttagccagccaacagcgtgaaaccctcagcgaactcagcgtcgaagaggtgttcaatcgccgtctggcactggaagaactggatgaatcgcagcagcaacgtctgcagcatcttttcaccacgacgttgcataccctcgccggagaacacgaagcatga

SEQ ID NO: 11 - DNA, 1428 bp, Escherichia coli
  source 1..1428: mol_type = genomic DNA
atgatgaatgacggtaagcaacaatctacctttttgtttcacgattacgaaacctttggcacgcaccccgcgttagatcgccctgcacagttcgcagccattcgcaccgatagcgaattcaatgtcatcggcgaacccgaagtcttttactgcaagcccgctgatgactatttaccccagccaggagccgtattaattaccggtattaccccgcaggaagcacgggcgaaaggagaaaacgaagccgcgtttgccgcccgtattcactcgctttttaccgtaccgaagacctgtattctgggctacaacaatgtgcgtttcgacgacgaagtcacacgcaacattttttatcgtaatttctacgatccttacgcctggagctggcagcatgataactcgcgctgggatttactggatgttatgcgtgcctgttatgccctgcgcccggaaggaataaactggcctgaaaatgatgacggtctaccgagctttcgccttgagcatttaaccaaagcgaatggtattgaacatagcaacgcccacgatgcgatggctgatgtgtacgccactattgcgatggcaaagctggtaaaaacgcgtcagccacgcctgtttgattatctctttacccatcgtaataaacacaaactgatggcgttgattgatgttccgcagatgaaacccctggtgcacgtttccggaatgtttggagcatggcgcggcaataccagctgggtggcaccgctggcgtggcatcctgaaaatcgcaatgccgtaattatggtggatttggcaggagacatttcgccattactggaactggatagcgacacattgcgcgagcgtttatataccgcaaaaaccgatcttggcgataacgccgccgttccggttaagctggtgcatatcaataaatgtccggtgctggcccaggcgaatacgctacgcccggaagatgccgaccgactgggaattaatcgtcagcattgcctcgataacctgaaaattctgcgtgaaaatccgcaagtgcgcgaaaaagtggtggcgatattcgcggaagccgaaccgtttacgccttcagataacgtggatgcacagctttataacggctttttcagtgacgcagatcgtgcagcaatgaaaattgtgctggaaaccgagccgcgtaatttaccggcactggatatcacttttgttgataaacggattgaaaagctgttgttcaattatcgggcacgcaacttcccggggacgctggattatgccgagcagcaacgctggctggagcaccgtcgccaggtcttcacgccagagtttttgcagggttatgctgatgaattgcagatgctggtacaacaatatgccgatgacaaagagaaagtggcgctgttaaaagcactttggcagtacgcggaagagattgtctaa

SEQ ID NO: 12 - DNA, 3543 bp, Escherichia coli
  source 1..3543: mol_type = genomic DNA
atgagtgatgtcgccgagacactagatcctttgcgcttgcccttacagggtgagcgcctgattgaagcctctgccggcacaggcaaaacctttacgattgcggcgctctatttgcgcctgttacttggactaggcggttccgccgcctttccccgcccgctgaccgttgaagaactgctggtggtcacctttaccgaggctgccacggcagaattgcgcggtcgtatccgtagcaatatccacgagttgcgcatcgcctgtctgcgtgaaaccaccgacaatccactgtacgaacgcctgctggaagagatcgacgataaagcgcaagccgcgcagtggttgttgttagccgaacggcagatggatgaagcggcagtctttactattcacggcttttgccagcgcatgctcaacctgaatgcctttgaatccggcatgctgtttgagcagcagctgattgaagatgagtctctgctacgctaccaggcctgcgccgatttctggcgtcgccactgctacccgctgccgcgtgaaatagcccaggtcgtctttgaaacctggaaagggccgcaggcgttgctgcgcgatattaatcgttatctgcaaggcgaagcgccggttatcaaagcaccgccgcccgatgatgaaacgctggcttcccgtcacgcgcaaattgtggcgcgtattgatacggtaaaacagcagtggcgcgacgcagtgggtgaactggatgcgctgatcgaatcttctggtattgatcgacgcaagtttaaccgtagcaatcaggctaaatggatcgacaagatcagcgcctgggcagaagaagagacaaacagttatcagttgccggagtcgctggaaaaattctcccagcgtttcttagaagatcgcacgaaggccgggggggaaaccccgcgacatccactgtttgaggcgatcgatcaactgcttgcagaaccattgtcgatccgcgatctggtgatcacccgcgcattggctgagatccgcgaaacagtagcgcgtgaaaaacgccgccgtggcgaattgggttttgatgacatgttaagtcggctcgattccgcgctgcgtagcgaaagcggtgaggtgttggcagcggcgatccgtacgcgattcccggtggcaatgatcgatgaatttcaggataccgacccccagcagtaccgaatttttcgccgtatctggcaccatcagccggaaaccgcattgttgctaattggcgacccgaagcaggccatatatgcattccggggtgcggatatcttcacttatatgaaggcgcgtagcgaagttcacgcccactacactttagacaccaactggcgttccgcaccaggaatggtgaacagcgtgaataagcttttcagccagactgatgacgcgttcatgtttcgcgaaataccgtttattccagtgaaatcagccgggaaaaatcaggcgttacgttttgtatttaaaggtgaaacacagcctgcgatgaaaatgtggctgatggaaggcgaaagctgcggcgttggcgattatcaaagtaccatggcgcaggtatgtgctgcgcaaatccgcgactggctacaagccggacagcggggcgaagcgttgctgatgaacggcgacgacgcgcgtccggtgcgtgcttcggacatcagtgtgctggtgcgcagccgccaggaggccgcccaggtgcgcgatgccttaacgttgctggaaatcccttccgtttacctttcgaaccgcgacagtgtttttgaaactctggaagcgcaggaaatgctttggttgttgcaggcggtgatgacgcccgaacgtgagaacaccctgcgtagtgcgctggcaacgtcaatgatggggctgaacgcgctggatatcgaaacgctgaacaatgacgaacatgcgtgggatgtggtagtcgaagagttcgatggttatcggcaaatctggcgcaaacgtggcgttatgccgatgctgcgggcgctgatgtcggcgcgtaacattgctgaaaacttgctggcaacggcaggcggtgagcggcgtcttaccgatatcttgcatatcagcgaactgctacaagaagccggaacgcagctggaaagtgaacatgcgctggtacgctggttatcgcaacatatcctcgagccagacagtaatgcctccagccaacaaatgcgtctcgaaagtgataaacatctggtgcagattgtcacgatccacaaatcgaaagggctggaatatccattggtctggctgccgtttatcaccaatttccgcgtccaggagcaggcgttttatcacgatcgccactcgtttgaggcagttctggatcttaatgctgcgccagaaagcgtcgacctcgcggaggccgaacgtctggcggaagatctgcgtttgctttacgtggcgctgacacgttcggtttggcattgcagtctcggcgttgcaccgctggtgcgccgtcgtggcgataaaaaaggtgacaccgacgtccaccaaagtgcgctcgggcgtttgctgcaaaaaggggaaccgcaagatgcggcagggcttcgcacctgtattgaagcgttatgcgatgatgatattgcctggcaaacggcacaaactggtgataaccaaccctggcaggttaatgatgtttctacagcagagctgaatgcgaagacgttacaacgattgcccggcgataactggcgcgtcaccagctactctggtttgcaacagcgtggtcacggtatcgcccaggatttgatgcctcggctggatgtcgatgctgcaggcgttgccagcgtcgttgaagaaccgacgttaacaccacatcagtttccgcgcggtgcgtcaccggggacgttcttgcacagtttgtttgaagacctggattttacccagccggttgacccgaactgggtgcgggaaaaactggaactcggcggctttgaatcgcagtgggaaccggtattgaccgagtggatcacggctgtcctccaggcacctctcaatgaaaccggcgtaagcctgagtcaactttccgcccgcaataaacaggtggagatggagttttatctgccgattagtgaaccgcttatcgccagtcagcttgatacgttaatccgccagtttgacccgctatccgcaggctgcccgccgctggagttcatgcaggtacgtggcatgttaaaaggctttatcgacctggtgttccgccacgaagggcgttattacctgctcgactataaatccaactggttgggtgaagacagttcggcttacacccaacaggctatggcagcggcaatgcaggcacaccgctatgatctgcaatatcagctttataccctggcgctgcatcgttatctgcgccatcgcattgctgattacgactatgagcaccactttggcggcgttatttatctgttcctgcgtggcgttgataaagaacatccgcaacaggggatttacacaacccgacccaacgccgggttgattgccctgatggatgagatgtttgccggtatgaccctggaggaggcgtaa

SEQ ID NO: 13 - DNA, 1827 bp, Escherichia coli
  source 1..1827: mol_type = genomic DNA
atgaaattgcaaaagcaattactggaagctgtggagcacaaacagctacgcccgctggatgtgcaatttgccctgaccgtggcgggagatgaacatcctgccgtcaccctcgcggcggcactgttaagtcatgatgccggagagggacacgtttgtttgccgctttcacgactggaaaataacgaggcgtcgcatccgctgttggcgacctgtgtcagtgaaatcggtgagctacaaaattgggaagaatgcttgctggcttctcaagcggtcagcaggggagatgaacccacgccgatgatcctctgtggcgatcgtctttatttgaatcgcatgtggtgtaacgagcgcacagtggcacgctttttcaacgaagtgaatcatgccattgaggttgatgaagctctactggcgcaaaccctggacaaactttttccagtaagcgatgaaattaactggcaaaaagttgcggcggcagtggcgctgacgcggcggatctcggtgatttccggcggccctggcaccggtaaaacgaccaccgtagcgaagttgctggcagcgttaattcaaatggccgacggcgaacgctgccgtatccgtctggctgcaccaacgggtaaagctgccgcgcgcttaaccgaatctctcggcaaggctttgcgacagttaccgctgaccgatgaacaaaagaaacgcattccggaagatgccagcactttgcaccgattgctgggcgcgcagccgggtagccagcgtttacgtcatcatgccggtaacccgctgcatcttgatgtgctggtggtagatgaagcgtcaatgatcgatctgcctatgatgtcgagactgatcgacgccttgcccgatcatgcgcgagtgatctttctcggcgatcgtgatcaactggcctcggttgaggctggggctgtgctgggcgatatctgcgcttatgccaacgcgggctttaccgccgagcgtgccaggcagctaagccgcctgacggggactcacgttccggcaggaaccggcacagaagcggcatctttgcgcgacagtctctgcctgctgcaaaaaagctatcgtttcggcagcgattctggcattggtcagttagctgcggcgatcaaccgtggtgataaaacggcagtgaaaaccgtttttcagcaggattttactgatatcgaaaaacggcttttacagagcggcgaagattatattgcgatgcttgaggaagctcttgcgggttacggacgttatctggatctgctgcaagcgcgtgccgagccggatttaatcattcaggcgttcaatgagtaccagcttttgtgcgccctgcgggaagggccgtttggcgtggctggactgaatgagcgaattgagcagtttatgcaacagaagcgcaaaattcatcgtcatccgcactctcgttggtacgaaggtcgaccggtgatgattgcccgtaatgacagcgcgcttgggttgtttaatggcgatatcggtattgcgctggatcgcgggcaggggacgcgcgtctggtttgcgatgccggacggcaatattaagtctgtgcaaccgagtcgcttgccagagcacgaaactacgtgggcgatgacggtacataaatcgcagggatcggagttcgaccatgcggcgttgattttgccgagccaacgcacgccggtagtaacgcgagagctggtttataccgcggtgacccgcgcgcgtcgccgtctgtcgctgtatgccgatgagcgcatattaagtgcggcaatcgccactcgtactgagcggcgcagtggtctggcggcgttgtttagttcacgggaataa

SEQ ID NO: 14 - DNA, 1833 bp, Escherichia coli
  source 1..1833: mol_type = genomic DNA
gtgagtgatcagtttgacgcaaaagcgtttttaaaaaccgtaaccagccagccaggcgtttatcgcatgtacgatgctggtggtacggttatctatgtcggcaaagcgaaagacctgaaaaaacggctttccagctatttccgtagcaacctcgcttcgcgcaaaaccgaagcgctggtcgcccagatccagcaaattgatgtaacggttactcacacagaaaccgaagcgctgttgctggaacacaactacatcaaactctatcagccgcgttacaacgttttgctacgcgatgataaatcatatccttttatcttcctgagtggtgatacccacccgcgtctggcgatgcatcgtggtgcgaagcatgccaaaggtgaatatttcggcccgttcccgaatggctatgccgtacgtgaaacactggcgctactgcaaaagattttccccattcgccagtgcgaaaatagtgtttatcgcaatcgctcgcgtccgtgtctgcaataccagatagggcgctgtctgggaccgtgcgttgaaggactggtgagtgaagaagaatacgctcagcaggtcgagtatgtgcgcctgtttttgtctggcaaagatgatcaggtgcttacgcaactcattagtcgtatggaaactgccagccagaatctggagtttgaagaagctgcacgtattcgcgaccaaattcaggcggtgcgacgcgtcaccgaaaaacaattcgtttccaataccggcgacgacctcgacgttattggtgtggcgttcgatgcgggcatggcttgtgtccacgtattgttcattcgtcagggcaaagtgctcggcagccgcagctatttcccgaaagtgcctggcggtacggaactgagcgaggtggtagaaaccttcgtaggccagttctatttacaaggcagccagatgcgcaccttaccgggtgagatcctgctcgattttaatcttagcgataaaacgctgctcgccgattccctttcagaactggcgggacgcaagattaatgttcaaaccaaacctcgcggcgatagggcgcgttatctgaaactcgcgcgcaccaatgcggcgacggccttaaccagcaaactttcgcagcaatctaccgttcaccagcgactgaccgcgcttgccagcgtgttgaaattgccggaagtgaagcggatggagtgctttgacatcagccataccatgggcgaacaaaccgtcgcttcctgtgtggtgtttgatgctaacggcccgctgcgtgcggagtatcggcgctataacattacaggcatcacgccgggcgatgattatgcggcgatgaatcaggtgctgcgtcggcgttatggtaaagccattgacgacagtaagatcccggatgtgatccttatcgacggcggcaaaggccagcttgcgcaggcgaaaaatgtcttcgccgaactggatgtctcatgggataaaaatcatccgctgctacttggcgttgccaaaggagcagatcgtaaggctggactggaaacgctgttctttgagccggaaggtgagggatttagtttgccgccagattcacccgcgctgcatgttatccagcatattcgcgatgaatcacatgatcacgcgattggcgggcaccgtaaaaaacgggcgaaggtcaaaaataccagttccctggaaaccattgaaggcgtcgggccaaaacgtcggcaaatgttgttgaaatatatgggcggtttgcaaggtttacgtaacgccagcgtcgaggaaattgcaaaagtgccgggtatttcgcaaggtctggcagaaaagatcttctggtcgttgaaacattga

SEQ ID NO: 15 - DNA, 834 bp, Escherichia coli
  source 1..834: mol_type = genomic DNA
atgcatgtttttgataataatggaattgaactgaaagctgagtgttcgataggtgaagaggatggtgtttatggtctaatccttgagtcgtgggggccgggtgacagaaacaaagattacaatatcgctcttgattatatcattgaacggttggttgattctggtgtatcccaagtcgtagtatatctggcgtcatcatcagtcagaaaacatatgcattctttggatgaaagaaaaatccatcctggtgaatattttactttgattggtaatagcccccgcgatatacgcttgaagatgtgtggttatcaggcttattttagtcgtacggggagaaaggaaattccttccggcaatagaacgaaacgaatattgataaatgttccaggtatttatagtgacagtttttgggcgtctataatacgtggagaactatcagagctttcacagcctacagatgatgaatcgcttctgaatatgagggttagtaaattaattaagaaaacgttgagtcaacccgagggctccaggaaaccagttgaggtagaaagactacaaaaagtttatgtccgagacccgatggtaaaagcttggattttacagcaaagtaaaggtatatgtgaaaactgtggtaaaaatgctccgttttatttaaatgatggaaacccatatttggaagtacatcatgtaattcccctgtcttcaggtggtgctgatacaacagataactgtgttgccctttgtccgaattgccatagagaattgcactatagtaaaaatgcaaaagaactaatcgagatgctttacgttaatataaaccgattacagaaataa

SEQ ID NO: 16 - DNA, 1380 bp, Escherichia coli
  source 1..1380: mol_type = genomic DNA
atggaatctattcaaccctggattgaaaaatttattaagcaagcacagcaacaacgttcgcaatccactaaagattatccaacgtcttaccgtaacctgcgagtaaaattgagtttcggttatggtaattttacgtctattccctggtttgcatttcttggagaaggtcaggaagcttctaacggtatatatcccgttattctctattataaagattttgatgagttggttttggcttatggtataagcgacacgaatgaaccacatgcccaatggcagttctcttcagacatacctaaaacaatcgcagagtattttcaggcaacttcgggtgtatatcctaaaaaatacggacagtcctattacgcctgttcccaaaaagtctcacagggtattgattacacccgatttgcctctatgctggacaacataatcaacgactataaattaatatttaattctggcaagagtgttattccacctatgtcaaaaactgaatcatactgtctggaagatgcgttaaatgatttgtttatccctgaaaccacaatagagacgatactcaaacgattaaccatcaaaaaaaatattatcctccaggggccgcccggcgttggaaaaacctttgttgcacgccgtctggcttacttgctgacaggagaaaaggctccgcaacgcgtcaatatggttcagttccatcaatcttatagctatgaggattttatacagggctatcgtccgaatggcgtcggcttccgacgtaaagacggcatattttacaatttttgtcagcaagctaaagagcagccagagaaaaagtatatttttattatagatgaaatcaatcgtgccaatctcagtaaagtatttggcgaagtgatgatgttaatggaacatgataaacgaggtgaaaactggtctgttcccctaacctactccgaaaacgatgaagaacgattctatgtcccggagaatgtttatatcatcggtttaatgaatactgccgatcgctctctggccgttgttgactatgccctacgcagacgattttctttcatagatattgagccaggttttgatacaccacagttccggaattttttactgaataaaaaagcagaaccttcatttgttgagtctttatgccaaaaaatgaacgagttgaaccaggaaatcagcaaagaggccactatccttgggaaaggattccgcattgggcatagttacttctgctgtgggttggaagatggcacctctccggatacgcaatggcttaatgaaattgtgatgacggatatcgcccctttactcgaagaatatttctttgatgacccctataaacaacagaaatggaccaacaaattattaggggactcatag

SEQ ID NO: 17 - DNA, 1047 bp, Escherichia coli
  source 1..1047: mol_type = genomic DNA
gtggaacagcccgtgatacctgtccgtaatatctattacatgcttacctatgcatggggttatttacaggaaattaagcaggcaaaccttgaagccatacccggtaacaatcttcttgatatcctggggtatgtattaaataaaggggttttacagctttcacgccgagggcttgagcttgattacaatcctaacaccgagatcattcctggcatcaaagggcgaatagagtttgctaaaacaatacgcggcttccatcttaatcatgggaaaaccgtcagtacttttgatatgcttaatgaagacacgctggctaaccgaattataaaaagcacattagccatattaattaagcatgaaaagttaaattcaactatcagagatgaagctcgttcactttatagaaaattaccgggcattagcactcttcatttaactccgcagcatttcagctatctgaatggcggaaaaaatacgcgttattataaattcgttatcagtgtctgcaaattcatcgtcaataattctattccaggtcaaaacaaaggacactaccgtttctatgattttgaaagaaacgaaaaagagatgtcattactttatcaaaagtttctttatgaattttgccgtcgtgaattaacgtctgcaaacacaacccgctcttatttaaaatgggatgcatcgagtatatcggatcagtcacttaatttgttacctcgaatggaaactgacatcaccattcgctcatcagaaaaaatacttatcgttgacgccaaatactataagagcattttttcacgacgaatgggaacagaaaaatttcattcgcaaaatctttatcaactgatgaattacttatggtcgttaaagcctgaaaatggcgaaaacataggggggttattaatatatccccacgtagataccgcagtgaaacatcgttataaaattaatggcttcgatattggcttgtgtaccgtcaatttaggtcaggaatggccgtgtatacatcaagaattactcgacattttcgatgaatatctcaaataa

SEQ ID NO: 18 - DNA, 1395 bp, Escherichia coli
  source 1..1395: mol_type = genomic DNA
atgagtgcggggaaattgccggaggggtgggttatcgccccagtatctacggtcacaactctaatccgaggagtaacgtataaaaaagagcaggcaataaattatctaaaagatgattatttgcctcttatccgtgcgaacaatattcagaatggcaagtttgatactacggacttggtttttgttcctaaaaatcttgttaaagaaagtcaaaaaatatctcctgaagatattgttattgcaatgtcatcagggagcaaatccgtagttggtaaatccgcacatcagcatctaccatttgaatgtagtttcggcgcattttgcggtgtattacgtcctgaaaaacttatattttctggttttattgctcatttcacaaaatcttctctttatcgaaacaaaatttcatcactttctgctggtgcaaatattaataatattaagccggcaagctttgatttgataaatataccaatcccaccacttgccgaacaaaaaatcatcgctgaaaaactcgatacgctgctggcgcaggtagacagcaccaaagcacgttttgagcaaatcccacaaatcctgaaacgttttcgtcaagcggtattggggggcgcagttaatggaaaattgacagaaaaatggcgtaattttgagccgcaacattctgtatttaagaagttaaattttgaatctatcttaactgaattacgtaatggtctttcatcaaagccaaatgaaagtggtgttggtcatccaatactacgcattagttctgtacgtgctggccatgtagatcaaaacgatattcggtttctagaatgttcagaaagtgaactaaaccgccacaaattacaagatggagatcttttatttactcgctataacggaagtttagaatttgttggtgtttgtgggttattgaaaaaattacaacatcaaaatttgctatatcctgataaacttattcgagctcgattaaccaaagatgctttaccagaatatatcgaaatatttttttcatccccctcagcacgaaatgcaatgatgaactgcgtgaaaacaacttctggtcaaaaaggtatttcaggaaaagatatcaaatcccaagttgttttattacctccagtaaaagaacaagccgaaatcgttcgccgcgtcgagcaactcttcgcctacgccgacaccatagaaaaacaggtcaacaacgccttagcccgcgtcaacaacctgacgcaatccatcctggcaaaagcgttccgtggtgaacttaccgcccagtggcgggccgaaaacccggatttgatcagcggagaaaacagcgccgccgcgttgctggaaaaaatcaaagctgaacgcgcagctagcgggggtaaaaaagcctcacgtaaaaaatcctga

SEQ ID NO: 19 - DNA, 1590 bp, Escherichia coli
  source 1..1590: mol_type = genomic DNA
atgaacaataacgatctggtcgcgaagctgtggaagctgtgcgacaacctgcgcgatggcggcgtttcctatcaaaactacgtcaatgaactcgcctcgctgctgtttttgaaaatgtgtaaagagaccggtcaggaagcggaatacctgccggaaggttaccgctgggatgacctgaaatcccgcatcggccaggagcagttgcagttctaccgaaaaatgctcgtgcatttaggcgaagatgacaaaaagctggtacaggcagtttttcataatgttagtaccaccatcaccgagccgaaacaaataaccgcactggtcagcaatatggattcgctggactggtacaacggcgcgcacggtaagtcgcgcgatgacttcggcgatatgtacgaagggctgttgcagaagaacgcgaatgaaaccaagtctggtgcaggccagtacttcaccccgcgtccgctgattaaaaccattattcatctgctgaaaccgcagccgcgtgaagtggtgcaggacccggcggcaggtacggcgggctttttgattgaagccgaccgctatgttaagtcgcaaaccaatgatctggacgaccttgatggcgacacgcaggatttccagatccaccgcgcgtttatcggcctcgaactggtgcccggcacccgtcgtctggcactgatgaactgcctgctgcacgatattgaaggcaacctcgaccacggcggcgcaatccgtctgggcaacactctgggtagcgacggtgaaaacctgccgaaggcgcatattgtcgccactaacccgccgtttggcagcgccgcaggcaccaacattacccgcacctttgttcacccgaccagcaacaaacagttgtgctttatgcagcatattatcgaaacgctgcatcccggcggtcgtgcggcggtggtggtgccggataacgtgctgtttgaaggcggcaaaggcaccgacattcgtcgtgacctgatggataagtgtcatctgcacaccattctgcgtctgccgaccggtattttttacgctcagggcgtgaagaccaacgtgctgttctttaccaaagggacggtggcgaacccgaatcaggataagaactgtaccgatgatgtgtgggtgtatgacctgcgtaccaatatgccgagtttcggcaagcgcacaccgtttaccgacgagcatttgcagccgtttgagcgcgtgtatggcgaagacccgcacggtttaagcccgcgcactgaaggtgaatggagttttaacgccgaagagacggaagttgccgacagcgaagagaacaaaaacaccgaccagcatcttgctaccagccgctggcgcaagttcagccgtgagtggatccgcaccgcaaaatccgattcgctggatatctcctggctgaaagataaagacagtattgatgccgacagcctgccggagccggatgtattagcggcagaagcgatgggcgaactggtacaggcgctgtctgaactggatgcgctgatgcgtgaactgggggcgagcgatgaggccgatttgcagcgtcagttgctggaagaagcgtttggtggggtgaaggaatga

SEQ ID NO: 20 - DNA, 3513 bp, Escherichia coli
  source 1..3513: mol_type = genomic DNA
atgatgaataaatccaattttgaattcctgaagggcgtcaacgacttcacttatgccatcgcctgtgcggcggaaaataactacccggatgatcccaacacgacgctgattaaaatgcgtatgtttggcgaagccacagcgaaacatcttggtctgttactcaacatccccccttgtgagaatcaacacgatctcctgcgtgaactcggcaaaatcgcctttgttgatgacaacatcctctctgtatttcacaaattacgccgcattggtaaccaggcggtgcacgaatatcataacgatctcaacgatgcccagatgtgcctgcgactcgggttccgcctggctgtctggtactaccgtctggtcactaaagattatgacttcccggtgccggtgtttgtgttgccggaacgtggtgaaaacctctatcaccaggaagtgctgacgctaaaacaacagcttgaacagcaggtgcgagaaaaagcgcagactcaggcagaagtcgaagcgcaacagcagaagctggttgccctgaacggctatatcgccattctggaaggcaaacagcaggaaaccgaagcgcaaacccaggctcgccttgcggcactggaagcacagctcgccgagaagaacgcggaactggcaaaacagaccgaacaggaacgtaaggcttaccacaaagaaattaccgatcaggccatcaagcgcacactcaaccttagcgaagaagagagtcgcttcctgattgatgcgcaactgcgtaaagcaggctggcaggccgacagcaaaaccctgcgcttctccaaaggcgcacgtccggaacccggcgtcaataaagccattgccgaatggccgaccggaaaagatgaaacgggtaatcagggctttgcggattatgtgctgtttgtcggcctcaaacccatcgcggtggtagaggcgaaacgtaacaatatcgacgttcccgccaggctcaatgagtcgtatcgctacagtaaatgtttcgataatggcttcctgcgggaaaccttgcttgagcactactcaccggatgaagtgcatgaagcagtgccagagtatgaaaccagctggcaggacaccagcggcaaacaacggtttaaaatccccttctgctactcgaccaacgggcgcgaataccgcgcaacaatgaagaccaaaagcggcatctggtatcgcgacgtgcgtgatacccgcaatatgtcgaaagccttacccgagtggcaccgcccggaagagctgctggaaatgctcggcagcgaaccgcaaaaacagaatcagtggtttgccgataaccctggcatgagcgagctgggcctgcgttattatcaggaagatgccgtccgcgcggttgaaaaggcaatcgtcaaggggcaacaagagatcctgctggcgatggcgaccggtaccggtaaaacccgtacggcaatcgccatgatgttccgcctgatccagtcccagcgttttaaacgcattctcttccttgtcgaccgccgttctcttggcgaacaggcgctgggcgcgtttgaagatacgcgtattaacggcgacaccttcaacagcattttcgacattaaagggctgacggataaattcccggaagacagcaccaaaattcacgttgccaccgtacagtcgctggtgaaacgcaccctgcaatcagatgaaccgatgccggtggcccgttacgactgtatcgtcgttgacgaagcgcatcgcggctatattctcgataaagagcagaccgaaggcgaactgcagttccgcagccagctggattacgtctctgcctaccgtcgcattctcgatcacttcgatgcggtaaaaatcgctctcaccgccaccccggcgctacatactgtgcagattttcggcgagccggtttaccgttatacctaccgtaccgcggttatcgacggttttctgatcgaccaggatccgcctattcagatcatcacccgcaacgcgcaggagggggtttatctctccaaaggcgagcaggtagagcgcatcagcccgcagggagaagtgatcaatgacaccctggaagacgatcaggattttgaagtcgccgactttaaccgtggcctggtgatcccggcgtttaaccgcgccgtctgtaacgaactcaccaattatcttgacccgaccggatcgcaaaaaacgctggtcttctgcgtcaccaatgcccatgccgatatggtggtggaagagctgcgtgccgcgttcaagaaaaagtatccgcaactggagcacgacgcgatcatcaagatcaccggtgatgccgataaagacgcgcgcaaagtgcagaccatgatcacccgcttcaataaagagcggctgcccaatatcgtggtaaccgtcgacctgctgacgaccggcgtcgatattccgtcgatctgtaatatcgtgttcctgcgtaaagtacgcagccgcattctgtacgaacagatgaaaggccgcgccacgcgcttatgcccggaggtgaataaaaccagctttaagatttttgactgtgtcgatatctacagcacgctggagagcgtcgacaccatgcgtccggtggtggtgcgcccgaaggtggaactgcaaacgctggtcaatgaaattaccgattcagaaacctataaaatcaccgaagcggatggccgcagttttgccgagcacagccatgaacaactggtggcgaagctccagcgtatcatcggtctggccacgtttaaccgtgaccgcagcgaaacgatagataaacaggtgcgtcgtctggatgagctatgccaggacgcggcgggcgtgaactttaacggcttcgcctcgcgcctgcgggaaaaagggccgcactggagcgccgaagtctttaacaaactgcctggctttatcgcccgtctggaaaagctgaaaacggacatcaacaacctgaatgatgcgccgatcttcctcgatatcgacgatgaagtggtgagtgtaaaatcgctgtacggtgattacgacacgccgcaggatttcctcgaagcctttgactcgctggtgcaacgttccccgaacgcgcaaccggcattgcaggcagttattaatcgcccgcgcgatctcacccgtaaagggctggtcgagctacaggagtggtttgaccgccagcactttgaggaatcttccctgcgcaaagcatggaaagagacgcgcaatgaagatatcgccgcccggctgattggtcatattcgccgcgctgcggtgggcgatgcgctgaaaccgtttgaggaacgtgtcgatcacgcgctgacgcgcattaagggcgaaaacgactggagcagcgagcaattaagctggctcgatcgtttagcgcaggcgctgaaagagaaagtggtgctcgacgacgatgtcttcaaaaccggcaacttccaccgtcgcggcgggaaggcgatgctgcaaagaacctttgacgataatctcgataccctgctgggcaaattcagcgattatatctgggacgagctggcctga

SEQ ID NO: 21 - DNA, 915 bp, Escherichia coli
  source 1..915: mol_type = genomic DNA
atgacggttcctacctatgacaaatttattgaacctgttctgcgttatctggcaacaaaaccggaaggtgcagccgcgcgtgatgttcatgaggctgccgcggatgcattaggactggatgacagccagcgagcgaaagtcattaccagcggacaacttgtttataaaaatcgtgcaggctgggcgcatgaccgtttaaaacgtgccgggttgtcgcaaagtttgtcgcgtggcaaatggtgcctgactcctgcgggttttgactgggttgcgtctcatccccagccaatgacggagcaggagacgaaccatctggccttcgcttttgtgaatgtcaaacttaagtcacggccggatgccgtcgatttagatccgaaagccgactctcccgatcatgaagaacttgcaaagagcagcccggacgatcggttagatcaggcgctaaaagagcttcgtgatgcggtggctgatgaggttctggaaaacttattgcaggtttctccttcgcgctttgaagtcattgttctggatgttttgcatcgcctggggtatggcggccaccgtgatgatttgcagcgtgttggcggtactggagatggtggcatcgatggtgtgatatcgcttgataaacttggcctggagaaagtttatgttcaggcaaaacgttggcagaatactgtaggcaggccagaattacaggcattttacggcgcactggctgggcaaaaagcgaaacgtggggtgtttattaccacttctggatttacttctcaggcgcgtgactttgcccaatccgtcgagggtatggtgttggttgatggggaacgcctggtgcacttaatgatcgaaaacgaagtaggggtttcttcacgtttgttgaaggtgccgaaactggatatggactattttgagtga

SEQ ID NO: 22 - DNA, 784 bp, synthetic construct
  misc_feature 1..784: note = Description of Artificial Sequence: Syntheticpolynucleotide
  source 1..784: mol_type = other DNA
aagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttta

SEQ ID NO: 23 - DNA, 795 bp, synthetic construct
  misc_feature 1..795: note = Description of Artificial Sequence: Syntheticpolynucleotide
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 24 - DNA, 714 bp, synthetic construct
  misc_feature 1..714: note = Description of Artificial Sequence: Syntheticpolynucleotide
  source 1..714: mol_type = other DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 25 - DNA, 1422 bp, synthetic construct
  misc_feature 1..1422: note = Description of Artificial Sequence: Syntheticpolynucleotide
  source 1..1422: mol_type = other DNA
atgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatataaagttcctgagttcgattcgtccacaattaaaaatatctcttctgcaaaaggcctggacgtttgggacagctggccattacaaaacactgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgcctagcttcctgctgaacatcaaaggcaagaaaacatctgttgtcaaagacagcatccttgaacaaggacaattaacagttaacaaataa

SEQ ID NO: 26 - DNA, 918 bp, synthetic construct
  misc_feature 1..918: note = Description of Artificial Sequence: Syntheticpolynucleotide
  source 1..918: mol_type = other DNA
atgagactcaaggtcatgatggacgtgaacaaaaaaacgaaaattcgccaccgaaacgagctaaatcacaccctggctcaacttcctttgcccgcaaagcgagtgatgtatatggcgcttgctctcattgatagcaaagaacctcttgaacgagggcgagttttcaaaattagggctgaagaccttgcagcgctcgccaaaatcaccccatcgcttgcttatcgacaattaaaagagggtggtaaattacttggtgccagcaaaatttcgctaagaggggatgatatcattgctttagctaaagagcttaacctgatctctactgctaaaaactccagcgaagagttagatcttaacattattgagtggatagcttattcaaatgatgaaggatacttgtctttaaaattcaccagaaccatagaaccatatatctctagccttattgggaaaaaaaataaattcacaacgcaattgttaacggcaagcttacgcttaagtagccagtattcatcttctctttatcaacttatcaggaagcattactctaattttaagaagaaaaattattttattatttccgttgatgagttaaaggaagagttaatagcttatacttttgataaagatggaaatattgagtacaaataccctgactttcctatttttaaaagggatgtgttaaataaagccattgctgaaattaaaaagaaaacagaaatatcgtttgttggcttcactgttcatgaaaaagaagggagaaaaattagtaagctgaagttcgaatttgtcgttgatgaagatgaattttctggcgataaagatgatgaagctttttttatgaatttatctgaagctgatgcagcttttctcaaggtatttgatgaaaccgtacctcccaaaaaagctaaggggtga

SEQ ID NO: 27 - DNA, 912 bp, synthetic construct
  misc_feature 1..912: note = Description of Artificial Sequence: Syntheticpolynucleotide
  source 1..912: mol_type = other DNA
atgagactcaaggtcatgatggacgtgaacaaaaaaacgaaaattcgccaccgaaacgagctaaatcacaccctggctcaacttcctttgcccgcaaagcgagtgatgtatatggcgcttgctctcattgatagcaaagaacctcttgaacgagggcgagttttcaaaattagggctgaagaccttgcagcgctcgccaaaatcaccccatcgcttgcttatcgacaattaaaagagggtggtaaattacttggtgccagcaaaatttcgctaagaggggatgatatcattgctttagctaaagagcttaacctgactgctaaaaactccagcgaagagttagatcttaacattattgagtggatagcttattcaaatgatgaaggatacttgtctttaaaattcaccagaaccatagaaccatatatctctagccttattgggaaaaaaaataaattcacaacgcaattgttaacggcaagcttacgcttaagtagccagtattcatcttctctttatcaacttatcaggaagcattactctaattttaagaagaaaaattattttattatttccgttgatgagttaaaggaagagttaatagcttatacttttgataaagatggaaatattgagtacaaataccctgactttcctatttttaaaagggatgtgttaaataaagccattgctgaaattaaaaagaaaacagaaatatcgtttgttggcttcactgttcatgaaaaagaagggagaaaaattagtaagctgaagttcgaatttgtcgttgatgaagatgaattttctggcgataaagatgatgaagctttttttatgaatttatctgaagctgatgcagcttttctcaaggtatttgatgaaaccgtacctcccaaaaaagctaaggggtga

SEQ ID NO: 28 - DNA, 918 bp, synthetic construct
  misc_feature 1..918: note = Description of Artificial Sequence: Syntheticpolynucleotide
  source 1..918: mol_type = other DNA
atgagactcaaggtcatgatggacgtgaacaaaaaaacgaaaattcgccaccgaaacgagctaaatcacaccctggctcaacttcctttgcccgcaaagcgagtgatgtatatggcgcttgctctcattgatagcaaagaacctcttgaacgagggcgagttttcaaaattagggctgaagaccttgcagcgctcgccaaaatcaccccatcgcttgcttatcgacaattaaaagagggtggtaaattacttggtgccagcaaaatttcgctaagaggggatgatatcattgctttagctaaagagcttaacctgctgtctactgctaaaaactcccctgaagagttagatcttaacattattgagtggatagcttattcaaatgatgaaggatacttgtctttaaaattcaccagaaccatagaaccatatatctctagccttattgggaaaaaaaataaattcacaacgcaattgttaacggcaagcttacgcttaagtagccagtattcatcttctctttatcaacttatcaggaagcattactctaattttaagaagaaaaattattttattatttccgttgatgagttaaaggaagagttaatagcttatacttttgataaagatggaaatattgagtacaaataccctgactttcctatttttaaaagggatgtgttaaataaagccattgctgaaattaaaaagaaaacagaaatatcgtttgttggcttcactgttcatgaaaaagaagggagaaaaattagtaagctgaagttcgaatttgtcgttgatgaagatgaattttctggcgataaagatgatgaagctttttttatgaatttatctgaagctgatgcagcttttctcaaggtatttgatgaaaccgtacctcccaaaaaagctaaggggtga

SEQ ID NO: 29 - DNA, 918 bp, synthetic construct
  misc_feature 1..918: note = Description of Artificial Sequence: Syntheticpolynucleotide
  source 1..918: mol_type = other DNA
atgagactcaaggtcatgatggacgtgaacaaaaaaacgaaaattcgccaccgaaacgagctaaatcacaccctggctcaacttcctttgcccgcaaagcgagtgatgtatatggcgcttgctctcattgatagcaaagaacctcttgaacgagggcgagttttcaaaattagggctgaagaccttgcagcgctcgccaaaatcaccccatcgcttgcttatcgacaattaaaagagggtggtaaattacttggtgccagcaaaatttcgctaagaggggatgatatcattgctttagctaaagagcttaacctgccctttactgctaaaaactccagcgaagagttagatcttaacattattgagtggatagcttattcaaatgatgaaggatacttgtctttaaaattcaccagaaccatagaaccatatatctctagccttattgggaaaaaaaataaattcacaacgcaattgttaacggcaagcttacgcttaagtagccagtattcatcttctctttatcaacttatcaggaagcattactctaattttaagaagaaaaattattttattatttccgttgatgagttaaaggaagagttaatagcttatacttttgataaagatggaaatattgagtacaaataccctgactttcctatttttaaaagggatgtgttaaataaagccattgctgaaattaaaaagaaaacagaaatatcgtttgttggcttcactgttcatgaaaaagaagggagaaaaattagtaagctgaagttcgaatttgtcgttgatgaagatgaattttctggcgataaagatgatgaagctttttttatgaatttatctgaagctgatgcagcttttctcaaggtatttgatgaaaccgtacctcccaaaaaagctaaggggtga

SEQ ID NO: 30 - DNA, 45 bp, synthetic construct
  misc_feature 1..45: note = Description of Artificial Sequence: Syntheticoligonucleotide
  source 1..45: mol_type = other DNA
caaaagggcgctgttatctgataaggcttatctggtctcattttg

SEQ ID NO: 31 - DNA, 45 bp, synthetic construct
  misc_feature 1..45: note = Description of Artificial Sequence: Syntheticoligonucleotide
  source 1..45: mol_type = other DNA
caaaagggggctgttatctgataaggcttatctggtctcattttg

SEQ ID NO: 32 - DNA, 38 bp, synthetic construct
  misc_feature 1..38: note = Description of Artificial Sequence: Syntheticoligonucleotide
  source 1..38: mol_type = other DNA
ggcgctgttatctgataaggcttatctggtctcatttt

SEQ ID NO: 33 - DNA, 60 bp, synthetic construct
  misc_feature 1..60: note = Description of Artificial Sequence: Syntheticoligonucleotide
  source 1..60: mol_type = other DNA
ctgctcaaaaagacgccaaaagggcgctgttatctgataaggcttatctggtctcatttt

SEQ ID NO: 34 - protein, 297 aa, synthetic construct
  REGION 1..297: note = Description of Artificial Sequence: Syntheticpolypeptide
MSAVLQRFREKLPHKPYCTNDFAYGVRILPKNIAILARFIQQNQPHALYWLPFDVDRTGASIDWSDRNCPAPNITVKNPRNGHAHLLYALALPVRTAPDASASALRYAAAIERALCEKLGADVNYSGLICKNPCHPEWQEVEWREEPYTLDELADYLDLSASARRSVDKNYGLGRNYHLFEKVRKWAYRAIRQGWPVFSQWLDAVIQRVEMYNASLPVPLSPAECRAIGKSIAKYTHRKFSPEGFSAVQAARGRKGGTKSKRAAVPTSARSLKPWEALGISRATYYRKLKCDPDLAK

SEQ ID NO: 35 - protein, 297 aa, synthetic construct
  REGION 1..297: note = Description of Artificial Sequence: Syntheticpolypeptide
MSAVLQRFREKLPHKPYCTNDFAYGVRILPKNIAILARFIQQNQPHALYWLPFDVDRTGASIDWSDRNCPAPNITVKNPRNGHAHLLYALALPVRTAPDASASALRYAAAIERALCEKLGADVNYSGLICKNPCHPEWQEVEWREEPYTLDELADYLDLSASARRSVDKNYGLGRNYHLFEKVRKWAYRAIRQDWPVFSQWLDAVIQRVEMYNASLPVPLSPAECRAIGKSIAKYTHRKFSPEGFSAVQAARGRKGGTKSKRAAVPTSARSLKPWEALGISRATYYRKLKCDPDLAK

SEQ ID NO: 36 - protein, 264 aa, synthetic construct [sequence omitted]

SEQ ID NO: 37 - protein, 237 aa, synthetic construct
  REGION 1..237: note = Description of Artificial Sequence: Syntheticpolypeptide
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 38 - protein, 473 aa, synthetic construct
  REGION 1..473: note = Description of Artificial Sequence: Syntheticpolypeptide
MNIKKFAKQATVLTFTTALLAGGATQAFAKETNQKPYKETYGISHITRHDMLQIPEQQKNEKYKVPEFDSSTIKNISSAKGLDVWDSWPLQNTDGTVANYHGYHIVFALAGDPKNADDTSIYMFYQKVGETSIDSWKNAGRVFKDSDKFDANDSILKDQTQEWSGSATFTSDGKIRLFYTDFSGKHYGKQTLTTAQVNVSASDSSLNINGVEDYKSIFDGDGKTYQNVQQFIDEGNYSSGDNHTLRDPHYVEDKGHKYLVFEANTGTEDGYQGEESLFNKAYYGKSTSFFRQESQKLLQSDKKRTAELANGALGMIELNDDYTLKKVMKPLIASNTVTDEIERANVFKMNGKWYLFTDSRGSKMTIDGITSNDIYMLGYVSNSLTGPYKPLNKTGLVLKMDLDPNDVTFTYSHFAVPQAKGNNVVITSYMTNRGFYADKQSTFAPSFLLNIKGKKTSVVKDSILEQGQLTVNK

SEQ ID NO: 39 - protein, 305 aa, synthetic construct
  REGION 1..305: note = Description of Artificial Sequence: Syntheticpolypeptide
MRLKVMMDVNKKTKIRHRNELNHTLAQLPLPAKRVMYMALALIDSKEPLERGRVFKIRAEDLAALAKITPSLAYRQLKEGGKLLGASKISLRGDDIIALAKELNLISTAKNSSEELDLNIIEWIAYSNDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAKG

SEQ ID NO: 40 - protein, 303 aa, synthetic construct
  REGION 1..303: note = Description of Artificial Sequence: Syntheticpolypeptide
MRLKVMMDVNKKTKIRHRNELNHTLAQLPLPAKRVMYMALALIDSKEPLERGRVFKIRAEDLAALAKITPSLAYRQLKEGGKLLGASKISLRGDDIIALAKELNLTAKNSSEELDLNIIEWIAYSNDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAKG

SEQ ID NO: 41 - protein, 305 aa, synthetic construct
  REGION 1..305: note = Description of Artificial Sequence: Syntheticpolypeptide
MRLKVMMDVNKKTKIRHRNELNHTLAQLPLPAKRVMYMALALIDSKEPLERGRVFKIRAEDLAALAKITPSLAYRQLKEGGKLLGASKISLRGDDIIALAKELNLPFTAKNSSEELDLNIIEWIAYSNDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAKG

SEQ ID NO: 42 - protein, 305 aa, synthetic construct
  REGION 1..305: note = Description of Artificial Sequence: Syntheticpolypeptide
MRLKVMMDVNKKTKIRHRNELNHTLAQLPLPAKRVMYMALALIDSKEPLERGRVFKIRAEDLAALAKITPSLAYRQLKEGGKLLGASKISLRGDDIIALAKELNLLSTAKNSPEELDLNIIEWIAYSNDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAKG

SEQ ID NO: 43 - DNA, 281 bp, synthetic construct
  misc_feature 1..281: note = Description of Artificial Sequence: Syntheticpolynucleotide
  source 1..281: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatc

SEQ ID NO: 44 - DNA, 281 bp, synthetic construct
  misc_feature 1..281: note = Description of Artificial Sequence: Syntheticpolynucleotide
  source 1..281: mol_type = other DNA
ggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatc

SEQ ID NO: 45 - DNA, 260 bp, synthetic construct
  misc_feature 1..260: note = Description of Artificial Sequence: Syntheticpolynucleotide
  source 1..260: mol_type = other DNA
aaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacattaaacatgagagcttagtacatactatcaacaggttgaactgctgatc

SEQ ID NO: 46 - DNA, 389 bp, synthetic construct
  misc_feature 1..389: note = Description of Artificial Sequence: Syntheticpolynucleotide
  source 1..389: mol_type = other DNA
tgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatc

SEQ ID NO: 47 - DNA, 139 bp, synthetic construct
  misc_feature 1..139: note = Description of Artificial Sequence: Syntheticpolynucleotide
  source 1..139: mol_type = other DNA
gtagaattggtaaagagagtcgtgtaaaatatcgagttcgcacatcttgttgtctgattattgatttttggcgaaaccatttgatcatatgacaagatgtgtatctaccttaacttaatgattttgataaaaatcatta

SEQ ID NO: 48 - DNA, 69 bp, synthetic construct
  misc_feature 1..69: note = Description of Artificial Sequence: Syntheticoligonucleotide
  source 1..69: mol_type = other DNA
tcgcacatcttgttgtctgattattgatttttggcgaaaccatttgatcatatgacaagatgtgtatct

SEQ ID NO: 49 - DNA, 139 bp, synthetic construct
  misc_feature 1..139: note = Description of Artificial Sequence: Syntheticpolynucleotide
  source 1..139: mol_type = other DNA
gtagaattggtaaagagagttgtgtaaaatattgagttcgcacatcttgttgtctgattattgatttttggcgaaaccatttgatcatatgacaagatgtgtatctaccttaacttaatgattttgataaaaatcatta

SEQ ID NO: 50 - DNA, 466 bp, synthetic construct
  misc_feature 1..466: note = Description of Artificial Sequence: Syntheticpolynucleotide
  source 1..466: mol_type = other DNA
gctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacc

SEQ ID NO: 51 - DNA, 439 bp, synthetic construct
  misc_feature 1..439: note = Description of Artificial Sequence: Syntheticpolynucleotide
  source 1..439: mol_type = other DNA
gctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtacc

SEQ ID NO: 52 - DNA, 565 bp, synthetic construct
  misc_feature 1..565: note = Description of Artificial Sequence: Syntheticpolynucleotide
  source 1..565: mol_type = other DNA
gaattcgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggagctagcattgggtcatcggatcccgggcccgtcgactgcagaggcctgcatgcaagctt

SEQ ID NO: 53 - DNA, 584 bp, synthetic construct
  misc_feature 1..584: note = Description of Artificial Sequence: Syntheticpolynucleotide
  source 1..584: mol_type = other DNA
gaattcgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtcccgggcgctagttattaatattgggtcatcggatcccgggcccgtcgactgcagaggcctgcatgcaagctt

SEQ ID NO: 54 - DNA, 557 bp, synthetic construct
  misc_feature 1..557: note = Description of Artificial Sequence: Syntheticpolynucleotide
  source 1..557: mol_type = other DNA
gaattcgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccgagctcggatcccgggcccgtcgactgcagaggcctgcatgcaagctt

SEQ ID NO: 55 - DNA, 557 bp, synthetic construct
  misc_feature 1..557: note = Description of Artificial Sequence: Syntheticpolynucleotide
  source 1..557: mol_type = other DNA
aagcttgcatgcaggcctctgcagtcgacgggcccgggatccgagctcggtacctcgcgaatgcatctaggggacggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccgagctcgaattc

SEQ ID NO: 56 - DNA, 503 bp, synthetic construct
  misc_feature 1..503: note = Description of Artificial Sequence: Syntheticpolynucleotide
  source 1..503: mol_type = other DNA
ggcgccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccacatgtcctgcagaggcctgcatgcaagctt

SEQ ID NO: 57 - DNA, 539 bp, synthetic construct
  misc_feature 1..539: note = Description of Artificial Sequence: Syntheticpolynucleotide
  source 1..539: mol_type = other DNA
gaattctgcagatatccatcacactggcggccgctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccgggccccccctcgatcgaggtcgacggtatcgggggagctc

SEQ ID NO: 58 - DNA, 500 bp, synthetic construct
  misc_feature 1..500: note = Description of Artificial Sequence: Syntheticpolynucleotide
  source 1..500: mol_type = other DNA
gcgcgcagccttaattaagctagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccttaattaactgcgcgc

SEQ ID NO: 59 - DNA, 530 bp, synthetic construct
  misc_feature 1..530: note = Description of Artificial Sequence: Syntheticpolynucleotide
  source 1..530: mol_type = other DNA
aagcttgcatgcaggcctctgcagtcgacgggccctctagactcgagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccgctagcggccgtcccctagatgcattcgcgaggtaccgagctcgaattc

SEQ ID NO: 60 - DNA, 303 bp, synthetic construct
  misc_feature 1..303: note = Description of Artificial Sequence: Syntheticpolynucleotide
  source 1..303: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgttaaacatgagagcttagtacgtactatcaacaggttgaactgctgatc

SEQ ID NO: 61 - protein, 304 aa, synthetic construct
  REGION 1..304: note = Description of Artificial Sequence: Syntheticpolypeptide
MRLKVMMDVNKKTKIRHRNELNHTLAQLPLPAKRVMYMALALIDSKEPLERGRVFKIRAEDLAALAKITPSLAYRQLKEGGKLLGASKISLRGDDIIALAKELNLLSTAKNSSEELDLNIIEWIAYSNDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAK

SEQ ID NO: 62 - protein, 304 aa, synthetic construct
  REGION 1..304: note = Description of Artificial Sequence: Syntheticpolypeptide
MRLKVMMDVNKKTKIRHRNELNHTLAQLPLPAKRVMYMALALIDSKEPLERGRVFKIRAEDLAALAKITPSLAYRQLKEGGKLLGASKISLRGDDIIALAKELNLLSTAKNSPEELDLNIIEWIAYSNDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAK

SEQ ID NO: 63 - DNA, 1040 bp, synthetic construct
  misc_feature 1..1040: note = Description of Artificial Sequence: Syntheticpolynucleotide
  source 1..1040: mol_type = other DNA
  CDS 3..1025
tcatgaagaaacctgaactgacagcaacttctgttgagaagtttctcattgaaaaatttgattctgtttctgatctcatgcagctgtctgaaggtgaagaaagcagagccttttcttttgatgttggaggaagaggttatgttctgagggtcaattcttgtgctgatggtttttacaaagacagatatgtttacagacactttgcctctgctgctctgccaattccagaagttctggacattggagaattttctgaatctctcacctactgcatcagcagaagagcacaaggagtcactctccaggatctccctgaaactgagctgccagctgttctgcaacctgttgctgaagcaatggatgccattgcagcagctgatctgagccaaacctctggatttggtccttttggtccccaaggcattggtcagtacaccacttggagggatttcatttgtgccattgctgatcctcatgtctatcactggcagactgtgatggatgacacagtttctgcttctgttgctcaggcactggatgaactcatgctgtgggcagaagattgtcctgaagtcagacacctggtccatgctgattttggaagcaacaatgttctgacagacaatggcagaatcactgcagtcattgactggtctgaagccatgtttggagattctcaatatgaggttgccaacatttttttttggagaccttggctggcttgcatggaacaacaaacaagatattttgaaagaagacacccagaactggctggttcccccagactgagagcctacatgctcagaattggcctggaccaactgtatcaatctctggttgatggaaactttgatgatgctgcttgggcacaaggaagatgtgatgccattgtgaggtctggtgctggaactgttggaagaactcaaattgcaagaaggtctgctgctgtttggactgatggatgtgttgaagttctggctgactctggaaacaggagaccctccacaagacccagagccaaggaatgaatattagctagc

SEQ ID NO: 64 - protein, 341 aa, synthetic construct
  REGION 1..341: note = Description of Artificial Sequence: Syntheticpolypeptide
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 65 - DNA, 1734 bp, Escherichia coli
  source 1..1734: mol_type = other DNA
gtgaaacaacagatacaacttcgtcgccgtgaagtcgatgaaacggcagacttgcccgctgaattgcctcccttgctgcgccgtttatacgccagccggggagtacgcagtgcgcaagaactggaacgcagtgttaaaggtatgctgccctggcagcaactgagcggcgtcgaaaaggccgttgagatcctttacaacgcttttcgcgaaggaacgcggattattgtggtcggtgatttcgacgccgacggcgcgaccagcacggctctaagcgtgctggcgatgcgctcgcttggttgcagcaatatcgactacctggtaccaaaccgtttcgaagacggttacggcttaagcccggaagtggtcgatcaggcccatgcccgtggcgcgcagttaattgtcacggtggataacggtatttcctcccatgcgggggttgagcacgctcgctcgttgggcatcccggttattgttaccgatcaccatttgccaggcgacacattacccgcagcggaagcgatcattaaccctaacttgcgcgactgtaatttcccgtcgaaatcactggcaggcgtgggtgtggcgttttatctgatgctggcgctgcgcacctttttgcgcgatcagggctggtttgatgagcgtaacatcgcaattcctaacctggcagaactgctggatctggtcgcgctggggacagtggcggacgtcgtgccgctggacgctaataatcgcattctgacctggcaggggatgagtcgcatccgagccggaaagtgccgtccggggattaaagcgctgcttgaagtggcaaaccgtgatgcacaaaaactcgccgccagcgatttaggttttgcgctggggccacgtctcaatgctgccggacgactggacgatatgtccgtcggtgtggcgctgttgttgtgcgacaacatcggcgaagcgcgcgtgctggcaaatgaactcgatgcgctaaaccagacgcgaaaagagatcgaacaaggaatgcaaattgaagccctgaccctgtgcgagaaactggagcgcagccgtgacacgctacccggcgggctggcaatgtatcaccccgaatggcatcagggcgttgtcggtattctggcttcgcgcatcaaagagcgttttcaccgtccggttatcgcgtttgcgccagcaggtgacggtacgctgaaaggttccggtcgctccattcaggggctgcatatgcgtgatgcgctggagcgattagacacactctaccctggcatgatgctgaagtttggcggtcatgcgatggcggcgggtttgtcgctggaagaggataaattcaaactctttcaacaacggtttggcgaactggttactgagtggctggacccttcgctattgcaaggcgaagtggtatcagacggtccgttaagcccggccgaaatgaccatggaagtggcgcagctgctgcgcgatgctggcccgtgggggcagatgttcccggagccgctgtttgacggtcatttccgtctgctgcaacagcggctggtgggcgaacgtcatttgaaggtgatggtcgaaccggtcggcggcggtccactgctggatggtattgcttttaatgtcgataccgccctctggccggataacggcgtgcgcgaagtgcaactggcttataagctcgatatcaacgagtttcgcggcaaccgcagcctgcaaattatcatcgacaatatctggccaatttag